SEQUENCE LISTING (18 sequences)

SEQ ID NO: 1 - DNA, 20 bp, synthetic construct
  source 1..20: mol_type = other DNA
atcgaatgtggagctgccat

SEQ ID NO: 2 - DNA, 20 bp, synthetic construct
  source 1..20: mol_type = other DNA
ggacaaaaccactcggcagt

SEQ ID NO: 3 - DNA, 26 bp, synthetic construct
  source 1..26: mol_type = other DNA
agctgaggaagtagggtgtgcgtggg

SEQ ID NO: 4 - DNA, 26 bp, synthetic construct
  source 1..26: mol_type = other DNA
ggcggcaggtccctcctcgcagggaa

SEQ ID NO: 5 - DNA, 26 bp, synthetic construct
  source 1..26: mol_type = other DNA
ccctcctcgcagggaagttggcaggg

SEQ ID NO: 6 - DNA, 26 bp, synthetic construct
  source 1..26: mol_type = other DNA
cctcgcagggaagttggcagggtgag

SEQ ID NO: 7 - DNA, 26 bp, synthetic construct
  source 1..26: mol_type = other DNA
ccctcctcgcagggaagttggcaggg

SEQ ID NO: 8 - DNA, 3886 bp, Homo sapiens
  source 1..3886: mol_type = genomic DNA
tgtactttccactatacactccattttcctaacatgctttttcaaaagaaaagaacaaattcttagtttgaggacaaaactgtcaaaatatacagattttattaacaaactgaccaatgaaaggcactgattcattgaaatattttagctatgattctgatcatatattaatttcaatgcataaaattatgaacattttaacatataataatggcacgtgtgaaaactactatcttcttgatgtatgtccattttttttcggttaatgtcatggttcattttatgtgttaacttggcttggccatggtgtccagatttggccaaacgttattctgtgtgtgtctctgatggtgtttcttggatgagattaacatttgaatcagtgggtcttgcataatgtcctctaggtccatccacgtgtcacaaatgacagaatttctaacttaaaggttgaacggcattctgatgtgtatatattccacgttttctttacccattcatccactgatggacagaaagagaaatagcaaataatctcacttatatgtggaatctgaaacaaactcataagagtagagagtacaatggtggttaccagaggctagagggtgaggtggggtggaatggtgaaatgttggtcaaaggatacaaagtttcagttagaaggaataagtttttgaaatctatttcacagcattgtgaatatagataataatgccttgcatatttcaaaattgctaaatttcaaatgttcctatcacaaaaatgatagacatgttaattagcttgatataatcattccacatggaataaaaatataaaaatatcacattgtatcccataaatatatacaattataatatgtcaattaaagataaatgtcaaaaagaaaatcggaataaaaactattaaaaaatcagtggacaatgtacaaagaagattattctccattctgcaggtaggcctcatccaatcagttgaaggccttaacagaacaaagactgatctaccctaagcaagaaggaattctgccagactgcatttggagttggactataacttttccctgaatctccaggctgcctgactgtaaattttgcactagtacctccacaataccttaaaactaaaatatatatatctatgtgcatacacacatctagctggctccgtttttctggagaacactgaataatgaagttatttatatgtcttttctttttttgaggcagagtctagctcttttgcccaggctggagttcagtggtgcaacctcagctcactgcaagctccacctcccgggttcaagtgattctcctgcctcagaaaaggtagctgggattacaggtgtgtaccaccaggactaattttttttttttttttcagtagagatgaggtctcaccatgttggccaaactggtctcgaactcctgaccccaactgactcgcctcagcttcccaaagtgctgggattacaggcatgagccaccgtgcctggcagttatttatatttatttgctcttatgtttccaggtttcttctaactttaaaattctatgactggaacaaatacatatgtgaacttggaagtagatctttcccctgtccagccttcagatcagaccacagccttagtcaccactccaattgtggcctcctgagagacactgagctaagggcatccaggttcttgacccacaaactgtgaaatacatgttgcttttaaaaaattccaagagtggcccttaaatttctttgggactataccgtgtaatagcgactgactcaaaagggtagtctggaaacctaatcttagcccctggtcacctctagctagtgtgcacagacctcagatcttgtcagtctcatttccctacctgttaacgtgagacagttgggcagaagaactttagcttcccttaaatgctacaactcagcgtctctattaatcataccgtttattcatcaaaatgaatactgataaaaatgacaagtagaacacaccctagatgttcaggcctctggaattggtggcagcgttccagcttcctttctgcctgtaactgtgagtaagtcctgctttacctccttcccaccccccgcacccaccaacagctctctatcataagctaatcagcttgaaataattgttagggcacttctgtgtatggcctgtgcatgtgccagtgtgtacaccgtgcaagctgagctgttgaggttctggatgtgcatacagccctggcaccggaaggtttgcctctagcattttgtggactaactctggcagtacacaatgggaagaaaacctctttccaagtgtgagcccttctctcttctcattcccaaggaggtgtgtccagatccctttgccagacagctactttacaggagcattggagagcaggcagctaactttccccaacaagcagctggatttacgttggcaaatcaatccctctcaataaaatgttatgtgaccatcttcccagccccccgttaaaatcccttcattttttcgagcaagcgaactagtcagagaagacagaaaagttaagtaaagaaagtggcagaaaagtgaagcaaagcaacagcccgggagcgtggcggggggacccgcgcggggactcgggtaggctgcaaacgctggggagatgccacaggagcgcctgccttcccagcttctcctttctctcgccgagggtctctggggttcagcgctcccggcgtttcggcgacctccgggtccgccctgtcccctcccagctggggagctgagcccggctctgcgcccatcgccgaccccggccactctcccttcgctgctcgctccaaaagcgcctgtcccgccccgcggggcgacttctccctttcggccctggggttgaagatcctgtgtggggcagctgcgccttcgggacagccccagccgcggagcgcccagaccagggcagccggagtcgggtcatacgccgcacgccgccggggccgccgcacctgagcccagctccagtcacgccggaagcgcgggcggagcgcacgggtccggggagagtcgcctgcgcccgcgagggggacccaagtacctagggtggtggccgagtcccgcctcccgccagcgggggcgaggacctgcgacgcgcacccctgcctggcccggtctcctcagcaccagccccacgcacaccctacttcctcagcttctcgccctcaccctgccaacttccctgcgaggagggacctgccgccagcctgcttcctcgtccgcaggccctgcgctgaacgctgccgcgcccagggttcaccttgcgccgtcgggaaagcccatgaactctccagaaacggcgtaaaggagggtcccgccgcggcgcagggctggggcgcctgggttccccctgggtggagcagcggcagcagagcgggaaagtggtggaggatgatcttgcggccaaaggggacctcggcgcagtaatgtcaacatgtaagtctcacctttcagcttggaattccttcactcttttatctgtgtccaccttctgtcgccccctccccgaccaccccaccctcaagagaaatgattttataaggacttcaactgcacggagggaataaaccggtttgtcttcagtttgcccctcgaggcactgaggagtaggactattgccaagtggagttgtgacatgtgtgtgtggctgttttttttcccccagcgaggtaaacctcacatttgggcggtgtaaagcctaataagaagggcagggcttcaagaatggggaaaacgtgttttcaattcaaattaaacaaggagtaacaggcaccgctcctcagtccagaggcctggccctgccaagaaggcgctctccggaatcaacacctgggggcttggaag

SEQ ID NO: 9 - DNA, 26 bp, synthetic construct
  source 1..26: mol_type = other DNA
cccgtaacatgggatctatggtgtag

SEQ ID NO: 10 - DNA, 26 bp, synthetic construct
  source 1..26: mol_type = other DNA
cccagaatgcaggaagtgcaccattc

SEQ ID NO: 11 - DNA, 26 bp, synthetic construct
  source 1..26: mol_type = other DNA
cccctgaattaaagacagtcacccag

SEQ ID NO: 12 - DNA, 26 bp, synthetic construct
  source 1..26: mol_type = other DNA
gtctatggtgaaaagagacccctgaa

SEQ ID NO: 13 - DNA, 26 bp, synthetic construct
  source 1..26: mol_type = other DNA
ctctaataccaaactttccaagctcc

SEQ ID NO: 14 - DNA, 4014 bp, Homo sapiens
  source 1..4014: mol_type = genomic DNA
tctccattttttttaaaatgaggatttatagtttgtttcaaatactgtgctgtctctggttcttctttgtgagactgtaaatgtgctattattggaagtagctaaatgcagaatattttgagaaataattctgtgtgataaaaattgtattaaaatagatctccacctaatattctccatttacacaagttaattctggaagcctttattttattataggactgattcaaacacaaaaagtagaagtttcattatcaactttaaatgtcatatgactaaatctaaggcagtgataatatatacattttagtcacagacataactctcaattaaaaaggagaagaaggagcctagtattcaaaagtaatttgtatatgatattggagggaagtttactgggaatttattcccacgctttgagcaaagtagcacccttatgatgactcaaggccacctcccaatttctcagacccatgcctgcccttctggcctgagaactcctgttacccacctgttcgtgtggtccatggcactcctacaggtggtggaaaggctgtgaagtgatgtcatttgaaagatatttccttgcaggatgttggaaagtagataaaccacaaccctgttccacaagggaactgcagagctttataagagctggtgatgttccctaagatcttctctcagtgttaggatagctagaaaggaccttcagggactccttgagaagtcttttatcagagaagaaattgagaaagtcaggtgtgtatgtttggtgtgggacagtgaagaataaggagagggaatggtgcacttcctgcattctgggtgactgtctttaattcaggggtctcttttcaccatagactgggacattattaagaagtcatagtatagaccattctaatatttgcctttttatgggacaatgttttgaagatgtcagcaaccaaaacaaatatggctatgttaataattgctaatacttatatttaaattgaacacacattttgtactaaatgtcattttaagcacttaacacatattggttgattacctacaccatagatcccatgttacgggttctataattgatcctattttgcaaataaggaacctgaggcacgtggagattaactaacttatccagtgtctcttaattagtaagtagccaagccaggattggagcttggaaagtttggtattagagctgtgctcttctaaccagtataaattagactgtcattcatcataaacaggattagatattagatttagtaccaggataggctaaagctcattcttgctcaggggagaagtatgaaggtccaagagccatctctctggccagcagtgcattctgtgagaaaacactaggtttgcagtgtgttaacatatttgtttcaaacaaataatatttgtttcccacaatacttcctggatacctaactatgaaaaagtatgattcagttttacagggattcctaagcttctctaagaatttctctagaccagaggttggcaaattaggagggcctgtaacaggccatgcatttttgtttacttgtttgtttagagacggggtttcactctgtcccccagggtggactgtagtggtgccatcagagatcgctataaccttgaacttctgggctcaagtgatcctcctacttcagctctcctgagtaggtaggacatcaggtgtgcaccaccatgcccagctaattttttaaaacatttattgtagagatgaggtctcactgtgttgtcaaggctggtcttgaactcctggccccatgtgatcctccctcctcagcctcccaaagtggtgggattacaggcatgagggattgtgcccagcacacatgtttttttaatacttgtttttctttttcttttcttttttttttttttttttttttttaagttggggtcttactgtgttgcccagactggcctcaaactcctgggctaaagcaatcttctaatctcagccttctgagtagctgggactaagctcccttgttcttataaataaagtttaattggaacacagccatgcccattagtttatatattgtctttgtctgcctctgtgctacaatggctgtgttaattgattgtgatcaatacagtatagcctgccatcctaaaatgtttacagtctagccctttaggagaacatttgccaccccctgaaactagtctaaagaactgagagtccgctttgctgaaccacatactttggacagcactagaaaagacctgctagatctcctcagccagggaaaacaaaccaatcacccacaggctagtttagccctacgacatgggtggtggtagtagcatttatttgttcctgttcatttacatttgtttcacacagtgcttttaattgatgaattatttccaatattaaacaattagatttcacataaaaatgaagttttggatttttctggtaaacagaatacactaggctactatatctgcatggccaacacagctggagctaaatggtggctgctgctttcaaaggagcttcccagtttactttagcccttagcaaccccttattcttcccactccagccccctttttgtctttagatggccatctggccctgatgggcttttaaatgggaatgctgatttatgccactccctttatacgggccaggaaactgaagcttagacaaaaaaaagtggcagaggcagttggtgggctccaacacgtgtctcctgtctccttgtctagtatttatgtctgggaatactcatttaaaaaattattttaaattattttaattgattgataagaactatatgtgtttatggtatacaacatgttttaatataggtatacattgtggaatggctaatatgagctaattaactttcattgttatctcatatacttatcattttatttgtagacagaacatttaaaatctctcagcaattttcaagtatacaatcattgttattaacaataatcatcatgttgtacaatagatttcttgaatgtatttctccagtctaactgaaattttccatcctttgacccatacattctcaactcctgtgccccatccacccctgtgcccctggaaaccactcttctattctttgcttttatgagttcaacctttttaggttccacatttaagtaagatcatgcagttctcataatttcttaaactctgtccctgagactcctttcccaagccttaagattatcggagtgcatcaaatacatactcatgtatcttagtcatctataagttttcaaagtaattttgtcaaaagggtaccgtgaacttaacggagtcaatatatttgagtctttctaacttttatagtgatcttcttgccattccaagttgacgttaaacactcacattccagacattcagatgtcctggactgtgtttcaggagaaatgatgggagagggaatttcctgctgcttaatcatcagctttgggagttgaagagtccaagtcactcattaactgacaggaaatacctgtaaacagagattatatcaaggcttgttagctaagcaaatctttgataagtgtaccctgttaacattttaacctttcattttttttccaggaaatgaaagcatcttgctgtttatggttataatttccaacaaactaatcagtacagtaaccaatgggagaatagaaatttatttctagcattccactgaatggacaatgtctgctaaatatgagcttttatttcaggattaatttgtgatggaaaaataaatagattttgcaaaggtatattgctaaataaattttagtacgaaagtaacatagtagaattgaactgactcaattcaacattaaaatatcctgtcatttttttcacatattaaacatcctagccttaataaaattgatatattattaataaatactctaacgattttagtatatgactttgttttcctatacttttctaaaaaatatttgggaattttaccttaattaggaaatttt

SEQ ID NO: 15 - DNA, 23 bp, synthetic construct
  source 1..23: mol_type = other DNA
tctaatatttgcctttttatggg

SEQ ID NO: 16 - DNA, 137 bp, synthetic construct
  source 1..137: mol_type = other DNA
ccatagactgggacattattaagaagtcatagtatagaccattctaatatttgcctttttatgggacaatattttgaagatgtcagcaaccaaaacaaatatggctatgttaataattgctaatacttatatttaaa

SEQ ID NO: 17 - DNA, 14 bp, synthetic construct
  source 1..14: mol_type = other DNA
gacaatgttttgaa

SEQ ID NO: 18 - DNA, 14 bp, synthetic construct
  source 1..14: mol_type = other DNA
gacaatattttgaa

(19) United States
(12) Patent Application Publication
(10) Pub. No.: US 2025/0367321 A1
(43) Pub. Date: Dec. 4, 2025

(54) COMPOSITIONS AND METHODS INVOLVING ADGRG6

(71) Applicant: The Regents of the University of California, Oakland, CA (US)

(72) Inventors: Nadav Ahituv, San Francisco, CA (US); Hai Nguyen, Oakland, CA (US)

(73) Assignee: The Regents of the University of California, Oakland, CA (US)

(21) Appl. No.: 18/877,637

(22) PCT Filed: Jun. 23, 2023
(86) PCT No.: PCT/US2023/026064

§ 371 Date: Dec. 20, 2024

Related U.S. Application Data
(60) Provisional application No. 63/355,414, filed on Jun. 24, 2022.

(51) Int. Cl. A61K 48/00; A61K 31/015; A61K 31/05; A61K 31/167; A61K 31/20; A61K 31/357; A61K 31/519; A61K 31/565; A61K 31/7088; A61K 38/46; A61P 3/04; C12N 9/22; C12N 15/113

(52) U.S. Cl. CPC A61K 48/0008; A61K 31/015; A61K 31/05; A61K 31/167; A61K 31/20; A61K 31/357; A61K 31/519; A61K 31/565; A61K 31/7088; A61K 38/465; A61P 3/04; C12N 9/226; C12N 15/113

(57) ABSTRACT

The present disclosure provides compositions and methods for reducing body fat in a human male subject by mutating or reducing the expression of the Adgrg6 gene in one or more cells of the human male subject.

### CROSS-REFERENCE TO RELATED PATENT APPLICATIONS

The present application claims benefit of priority to U.S. Provisional Patent Application No. 63/355,414, filed Jun. 24, 2022, which is incorporated by reference for all purposes.

### STATEMENT AS TO RIGHTS TO DISCLOSURES MADE UNDER FEDERALLY SPONSORED RESEARCH AND DEVELOPMENT

This invention was made with government support under R01 DK124769, and P01 HD084387 awarded by the National Institutes of Health. The government has certain rights in the invention.

### BACKGROUND OF THE DISCLOSURE

Obesity, an excess of white adipose tissue (WAT), is a global epidemic and is closely associated with chronic metabolic diseases, such as type 2 diabetes and cardiovascular disease [1, 2]. Obesity-related metabolic diseases are not simply the result of excess fat, but rather the distribution of adipose tissue has a major effect on these co-morbidities. While most studies have used clinical measures of overall fat and body mass index (BMI) to estimate disease risk, many studies using CT and MRI clearly show that abdominal visceral adipose tissue (VAT), but not subcutaneous adipose tissue (SAT), is associated with an increased risk for type 2 diabetes [3-9]. Additional studies also showed that increased VAT represents a risk factor for developing insulin resistance and cardiovascular disease [4-6, 10-15]. During periods of high metabolic activity, VAT release free fatty acids (FFA) that contribute more to plasma FFA than VAT [12]. As a less metabolic active organ, SAT has better long-term lipid storage capacity and is a buffer during intake of dietary lipids, protecting other tissues from lipotoxic effects [16]. In addition, SAT was found to be associated with increased HDL-cholesterol levels and decreased LDL-cholesterol levels [17-19], indicating its protective roles against cardiovascular disease. Upon cold or □-adrenergic stimuli, SAT can also acquire characteristics of brown adipose tissue (BAT), known as browning [20-23], becoming more metabolically active by dissipating energy via thermogenesis. Thus, increased SAT with browning potential correlates with insulin sensitivity [24-26].

Body fat distribution significantly differs between genders. This fat distribution difference is observed before puberty but becomes more prominent upon puberty [27, 28]. Following puberty, females predominantly accumulate SAT, while males amass significantly more VAT. Women accumulate fat in the hip and limbs while men accumulate a greater extent of fat in the trunk [29-31]. Accumulation of adipose tissue around the viscera, the body's internal organs, is associated with an increased risk of disease in both men and women [3-5, 32]. In contrast, the preferential accumulation of adipose tissue in the lower extremities, such as the hips and legs, has been suggested to contribute to a lower incidence of myocardial infarction and coronary death observed only in women during middle age [33, 34]. This difference is thought to be due to numerous genes that are differentially expressed in adipose tissue from obese males and females, with only a few located on sex chromosomes [35]. Many of these genes are involved in immune response and lipid and carbohydrate metabolism as well as clock genes, including PER2, BMAL2, and CRY1 [36]. The differential distribution of body fat between genders has been attributed to downstream effects of sex hormone secretion. Although sex steroids, especially estrogen, are involved in determining adipose distribution, several additional factors also play an important role. Recent human genome-wide association studies (GWAS) have identified multiple novel loci and pathways associated with measures of central obesity [37, 38]. A recent GWAS study on body fat distribution difference between genders identified multiple loci that are gender-heterogenous and associated with gender-specific fat distribution [39]. Many of these loci have marked gender dimorphic patterns, the majority of which have stronger associations in women than in men; however, the mechanisms underlying this dimorphism remain largely unknown.

Identifying and modifying one or more genes that can reduce body fat, especially VAT, can help to reduce the risk associated with chronic metabolic diseases, such as type 2 diabetes and cardiovascular diseases.

### BRIEF SUMMARY OF THE DISCLOSURE

In one aspect, the disclosure provides a method of reducing body fat in a human male subject, the method comprising mutating or reducing the expression of an Adgrg6 gene in one or more cells in the human male subject. In some embodiments, mutating the Adgrg6 gene comprises knocking out the gene or introducing a nucleotide deletion or insertion into the gene.

In certain embodiments, knocking out the Adgrg6 gene comprises introducing into the one or more cells of the human male subject a nuclease targeted to the Adgrg6 gene. In some embodiments, the nuclease is a RNA-guided nuclease, a zinc finger nuclease (ZFN), or a transcription activator-like effector nuclease (TALEN).

In certain embodiments, the RNA-guided nuclease is a clustered regularly interspaced short palindromic repeats (CRISPR) nuclease and the method further comprises introducing into the one or more cells of the human male subject a guide RNA (gRNA) that targets a portion of the Adgrg6 gene, wherein the gRNA comprises a sequence having at least 90%, 95%, 98%, 99% or 100% identity to a sequence of ATCGAATGTGGAGCTGCCAT (SEQ ID NO:1) or GGACAAAACCACTCGGCAGT (SEQ ID NO:2).

In some embodiments, reducing the expression of the Adgrg6 gene comprises CRISPR interference (CRISPRi), RNA interference (RNAi), or antisense therapy. In particular embodiments, the CRISPRi comprises introducing into the one or more cells of the human male subject a catalytically-inactive nuclease and a gRNA that targets a portion of a promoter or enhancer sequence operably linked to a coding sequence of the Adgrg6 gene. In certain embodiments, the catalytically-inactive nuclease is linked to a transcriptional repressor domain. In particular embodiments, the catalytically-inactive nuclease is dCas9. In some embodiments, the gRNA targets the promoter sequence comprising the sequence of SEQ ID NO: 8. In particular embodiments, the gRNA comprises a sequence having at least 90%, 95%, 98%, 99% or 100% identity to a sequence of AGCTGAGGAAGTAGGGTGTGCGTGGG (SEQ ID NO:3), GGCGGCAGGTCCCTCCTCGCAGGGAA (SEQ ID NO:4), CCCTCCTCGCAGGGAAGTTGGCAGGG (SEQ ID NO:5), CCTCGCAGGGAAGTTGGCAGGGTGAG (SEQ ID NO:6), or CCCTCCTCGCAGGGAAGTTGGCAGGG (SEQ ID NO:7).

In some embodiments, the gRNA targets the enhancer sequence comprising the sequence of SEQ ID NO:14. In certain embodiments, the gRNA comprises a sequence having at least 90%, 95%, 98%, 99% or 100% identity to a sequence of

(SEQ ID NO: 9)
CCCGTAACATGGGATCTATGGTGTAG,
(SEQ ID NO: 10)
CCCAGAATGCAGGAAGTGCACCATTC,
(SEQ ID NO: 11)
CCCCTGAATTAAAGACAGTCACCCAG,
(SEQ ID NO: 12)
GTCTATGGTGAAAAGAGACCCCTGAA,
or

In some embodiments, reducing the expression of the Adgrg6 gene comprises knocking in a single nucleotide polymorphism (SNP) proximal to the Adgrg6 gene in one or more cells of the human male subject, wherein the SNP is rs9403383. In certain embodiments, the knocking in comprises introducing into one or more cells of the human male subject a gRNA, an RNA-guided nuclease, and a homology-directed-repair template (HDRT) comprising the SNP rs9403383. In particular embodiments, the gRNA comprises a sequence having at least 90%, 95%, 98%, 99% or 100% identity to the sequence of TCTAATATTTGCCTTTTTATGGG (SEQ ID NO:15). In certain embodiments, the HDRT comprises a sequence having at least 90%, 95%, 98%, 99% or 100% identity to the sequence of

(SEQ ID NO: 16)
CCATAGACTGGGACATTATTAAGAAGTCATAGTATAGACCATTCTAATA
TTTGCCTTTTTATGGGACAATATTTTGAAGATGTCAGCAACCAAAACAA
ATATGGCTATGTTAATAATTGCTAATACTTATATTTAAA.

In another aspect, the disclosure features a method of reducing body fat in a human male subject, in which the method comprises reducing or blocking the activity of the Adhesion G-protein coupled receptor G6 (ADGRG6) protein in one or more cells in the human male subject. In some embodiments, the method comprises administering to the human male subject a small molecule that binds to the ADGRG6 protein. In particular embodiments, the small molecule is selected from the group consisting of valproic acid, 4-(5-benzo(1,3)dioxol-5-yl-4-pyridin-2-yl-1H-imidazol-2-yl)benzamide, dorsomorphin, tetrachlorodibenzodioxin, acetaminophen, benzo(a)pyrene, bisphenol A, estradiol, tretinoin, and trichostatin A. In particular embodiments, the small molecule is selected from the group consisting of alfuzosin, terazosin, clonidine, bisoprolol, betaxolol, metoprolol, atenolol, albuterol, nadolol, penbutolol, tolterodine, atropine, scopolamine, calcimar, metoclopramide, haloperidol, olanzapine, ropinirole, pramipexole, loratadine, cetirizine, demenhydrinate, cimetidine, ranitidine, trazodone, sumatriptan, exenatide, fentanyl, codein, meperidine, oxycodone, montelukast, misoprostol, clopidogrel, aripiprazole, quetiapine, montelukast, olanzapine, and valsartan.

In some embodiments, the human male subject has or is at risk of developing a metabolic disease. In certain embodiments, the metabolic disease is obesity, Type-1 diabetes, Type-2 diabetes, or a cardiovascular disease. In particular embodiments, the obesity is diet-induced obesity.

In some embodiments, the human male subject is overweight.

In some embodiments, the human male subject is undergoing a sex reassignment therapy to change into a female subject.

In some embodiments, the method reduces VAT in the human male subject.

In some embodiments, the method reduces body weight.

In some embodiments, the method does not reduce lean mass.

In some embodiments, the method reduces blood glucose.

In some embodiments, the method increases insulin sensitivity.

In some embodiments, the method comprises mutating or reducing the expression of an Adgrg6 gene in one or more cells ex vivo and then introducing the cells into the human male subject. In certain embodiments, the method further comprises before the mutating or reducing, isolating the cells from the human male subject. In certain embodiments, the cells are adipose stem cells or adipose progenitor cells.

In another aspect, the disclosure provides an isolated adipose cell having a mutated Adgrg6 gene or an Adgrg6 gene with reduced expression compared to a wild-type adipose cell. In some embodiments, the isolated adipose cell comprises a catalytically-inactive nuclease and a gRNA that targets a portion of a promoter or enhancer sequence operably linked to a coding sequence of the Adgrg6 gene. In certain embodiments, the catalytically-inactive nuclease is linked to a transcriptional repressor domain. In particular embodiments, the catalytically-inactive nuclease is dCas9.

In some embodiments of the isolated adipose cell, the gRNA targets the promoter sequence comprising the sequence of SEQ ID NO:8. In some embodiments, the gRNA comprises a sequence having at least 90%, 95%, 98%, 99% or 100% identity to a sequence of any one of SEQ ID NOS: 3-7.

In some embodiments of the isolated adipose cell, the gRNA targets the enhancer sequence comprising the sequence of SEQ ID NO:14. In some embodiments, the gRNA comprises a sequence having at least 90%, 95%, 98%, 99% or 100% identity to a sequence of any one of SEQ ID NOS: 9-13.

In some embodiments, the isolated adipose cell is an adipose stem cell or progenitor cell.

In another aspect, the disclosure provides a composition comprising a guide RNA (gRNA), wherein the gRNA comprises a sequence having at least 90%, 95%, 98%, 99% or 100% identity to a sequence of any one of SEQ ID NOS: 3-7 and 9-13. In some embodiments, the composition further comprises a catalytically-inactive nuclease. In some embodiments, the catalytically-inactive nuclease is linked to a transcriptional repressor domain. In particular embodiments, the catalytically-inactive nuclease is dCas9.

### BRIEF DESCRIPTION OF THE DRAWINGS

FIG. 1A: ADGRG6 expression in human subcutaneous adipose tissue (SAT) and visceral adipose tissue (VAT) of men and women obtained by GTEX portal.

FIG. 1B: Luciferase assays for ADGRG6_3 and ADGRG6_4 sequences cloned into pGL4.23 luciferase reporter vector in human preadipocytes. pGL4.13 (Promega) with an SV40 early enhancer was used as a positive control and the pGL4.23 empty vector as a negative control. Data are represented as mean±S.D***≤0.001.

FIG. 1C: Luciferase assays for ADGRG6_4 and associated variant containing rs9403383. Data are represented as mean±S.D ***≤0.001.

FIG. 1D: Transcription factor binding site analysis of unassociated and associates variant sequences showing HOXA3, GR, and PGR binding.

FIG. 1E: RT-qPCR of ChIP of HOXA3, GR, and PGR relative to input using unassociated and associated variant. IgG and PPARg are used as negative controls, and Histone H3 as a positive control. Data are represented as mean±S.D*≤0.05, **≤0.01, ***≤0.001.

FIG. 2A: qRT-PCR of ADGRG6, SOX9, PPARg, and FABP4 during adipocyte differentiation of human preadipocytes.

FIG. 2B: qRT-PCR of Adgrg6, Sox9, and Fabp4 in adipocytes and stromal vascular fraction (SVF) in mouse perigonadal white adipose tissue (pWAT). Data are represented as mean±S.D.**≤0.01, ***≤0.001.

FIG. 2C: qRT-PCR of Adgr6, CD109, CD34, and CD45 in mesenchymal stem cells (MSC), adipose progenitor cells (APC), and linage positive cells (Lin+), which were FACS-sorted from mouse pWAT. Data are represented as mean±S.D.*≤0.05, ***≤0.001.

FIG. 2D: qRT-PCR of Adgr6 in mouse inguinal white adipose tissue (iWAT), and pWAT of male and female C57BL/6J mice. Data are represented as mean±S.D. ***≤0.001.

FIG. 3A: Summary of examining adipogenic capacity in ADGRG6 enhancer knockout (EKO), ADGRG6 knockout (KO), ADGRG6 associated knockin (AKI), and ADGRG6 overexpression human preadipocytes.

FIG. 3B: qRT-PCR of ADGRG6 and adipogenic markers, including SOX9, C/EBPb, PPARg, and FABP4 during adipocyte differentiation of control, ADGRG6 EKO, ADGRG6 KO, and ADGRG6 AKI cells. Data are represented as mean±S.D*<0.05, **<0.01, ***<0.001.

FIG. 3C: Oil red O staining (left) and lipid distribution (right) by Fiji of control, ADGRG6 EKO, ADGRG6 KO, and ADGRG6 AKI cells.

FIG. 3D: (Left) qRT-PCR of ADGRG6 during adipocyte differentiation of control and ADGRG6 overexpression cells. (Right) Immunoblotting for ADGRG6. Data are represented as mean±S.D***≤0.001.

FIG. 3E: Oil red O staining (left) and lipid distribution (right) by Fiji of control, ADGRG6 overexpressing cells. Data are represented as mean±S.D**≤0.01, ***≤0.001.

FIG. 3F: qRT-PCR of SOX9, C/EBPb, C/EBPd, and FABP4 during human adipocyte differentiation. Data are represented as mean±S.D.*≤0.05, **≤0.01, ***≤0.001.

FIG. 4A: CAMP levels of control, ADGRG6 KO, ADGRG6 overexpression cells and ADGRG6 overexpression cells treated with 2′,5′-Dideoxyadenosine (dAdo) in basal condition or stimulated with forskolin. Data are represented as mean±S.D*≤0.05, **≤0.01, ***≤0.001.

FIG. 4B: Immunoblotting for phospho-CREB, CREB, and GAPDH in ADGRG6 KO in basal condition or stimulated with forskolin.

FIG. 4C: Immunoblotting for myc-ADGRG6, phospho-CREB, CREB, and GAPDH in ADGRG6 overexpression cells in basal condition or stimulated with forskolin.

FIG. 4D: Schematic of ADGRG6 action activating adipogenesis by stimulating cAMP and phospho-CREB.

FIG. 5A: Schematic of generation of Adgrg6 adipose-specific knockout mice, Adgrg6ASKO/ASKO and a summary of measured metabolic parameters, including body weight, fat mass by DEXA, adipogenic gene analysis by qRT-PCR, glucose tolerance test (GTT), and insulin tolerance test (ITT).

FIG. 5B: qRT-PCR of Adgrg6 in brown adipose tissue (BAT), inguinal adipose tissue (iWAT), and perigonadal adipose tissue (pWAT) of control Adgrg6fl/fl (male=9 mice, female=8) and Adgrg6ASKO/ASKO (male=9, female=6). Data are represented as mean±S.D*≤0.05, **≤0.01, ***≤0.001.

FIG. 5C: (Top) Body weight and (Bottom) mouse image of control (male=9 mice, female=8) and Adgrg6ASKO/ASKO (male=9, female=8) measured for 17 weeks. Data are represented as mean±S.D**≤0.01.

FIG. 5D: Fat and lean mass of control (male=9 mice, female=9) and Adgrg6ASKO/ASKO (male=10, female=8) measured by dual energy X-ray absorptiometry (DEXA). Data are represented as mean±S.D**≤0.01.

FIG. 5E: Whole-body scan images of control and Adgrg6ASKO/ASKO of both male and female mice.

FIG. 5F: Tissue weight of control (male=8 mice, female=8) and Adgrg6ASKO/ASKO (male=10, female=7). Data are represented as mean±S.D**≤0.01, ***≤0.001.

FIG. 5G: Glucose tolerance test (GTT) and insulin tolerance test (ITT) of male control (n=5 mice) and Adgrg6ASKO/ASKO (n=5). Data are represented as mean±S.D**≤0.01, ***≤0.001.

FIG. 6A: qRT-PCR of Sox9, Pparg, and Fabp4 male control and Adgrg6ASKO/ASKO mice. Data are represented as mean±S.D. *≤0.05, **≤0.01.

FIG. 6B: Body weight of male and female control and Adgrg6Adipoq-Cre mice. Data are represented as mean±S.D. **≤0.01.

FIG. 7A: Schematic of male Adgrg6ASKO/ASKO mice fed with high-fat diet (HFD) and metabolic phenotypes measured.

FIG. 7B: (Left) Body weight and (right) mouse image of control and Adgrg6ASKO/ASKO male and female mice measured for 16 weeks. Data are represented as mean #S.D***≤0.001.

FIG. 7C: Tissue weight (left) and tissue image (right) of male control (n=6 mice) and Adgrg6ASKO/ASKO (n=5). Data are represented as mean±S.D**≤0.01, ***≤0.001.

FIG. 7D: Glucose tolerance test (GTT) and insulin tolerance test (ITT) of male control (n=6) and Adgrg6ASKO/ASKO (n=5 mice). Data are represented as mean±S.D*≤0.05**≤0.01, ***≤0.001.

FIG. 8A: Schematic of generation of Adgrg6 adipose enhancer knockout, Adgrg6ARS-/- and a summary of measured phenotyping parameters, including body weight, fat mass, mRNA expression of adipogenic gene by qRT-PCR, GTT, and ITT.

FIG. 8B: qRT-PCR of Adgrg6 in brown adipose tissue (BAT), inguinal adipose tissue (iWAT), and perigonadal adipose tissue (pWAT) of control (male=8 mice, female=8) and Adgrg6ARS-/- (male=12, female=6). Data are represented as mean±S.D. ***≤0.001.

FIG. 8C: qRT-PCR of adipogenic markers, including SOX9, C/EBPb, PPARg, and FABP4 in BAT, iWAT, pWAT, liver, and muscle of control (male-8 mice, female=8) and Adgrg6ARS-/- (male=12, female=6). Data are represented as mean±S.D. *≤0.05, **≤0.01.

FIG. 8D: Body weight (left) and mouse image (right) of control (male=8 mice, female=8) and Adgrg6ASKO/ASKO (male=12, female=6) measured for 17 weeks. Data are represented as mean±S.D. *≤0.05.

FIG. 8E: (Left) fat and lean mass of control (male-6, female=7) and Adgrg6ASKO/ASKO (male=10, female=10) measured by dual energy X-ray absorptiometry (DEXA). (Right) whole-body scan images of control and Adgrg6ASKO/ASKO of both male and female mice. Data are represented as mean±S.D. *≤0.05.

FIG. 8F: tissue weight of control (male=7, female=7) and Adgrg6ASKO/ASKO (male=6, female=6). Data are represented as mean±S.D. **≤0.01, ***≤0.001.

FIG. 8G: Adgrg6^{ARS-/-} male mice on high-fat diet for 13 weeks.

FIG. 8H: Adgrg6^{ARS-/-} mice had lower body weight than littermate control mice.

FIG. 8I: Adgrg6^{ARS-/-} mice had smaller iWAT and pWAT.

FIG. 8J: Adgrg6^{ARS-/-} mice showed improved GTT and ITT compared to control males.

FIG. 9A: Cartoon of intravenous tail vein delivery of Adgrg6 AAV-CRISPRi, which consists of both AAV9-CMV-sadCas9-KRAB and AAV9-U6-sasgRNA-CMV-mCherry into 5 wk-old C57BL/6J mice and a summary of metabolic parameters, including body weight, fat mass, mRNA expression, GTT, and ITT.

FIG. 9B: qRT-PCR of Adgrg6 in brown adipose tissue (BAT), inguinal adipose tissue (iWAT), and perigonadal adipose tissue (pWAT), kidney, liver, and heart of control mice (dCas9-Krab) (n=6), CRISPRi targeting promoter (n=6), or enhancer (n=6) of Adgrg6 mice. Data are represented as mean±S.D. *≤0.05, ***≤0.001.

FIG. 9C: Body weight (left) and mouse image (right) of control (n=6) and Adgrg6ASKO/ASKO (n=6) measured for 12 weeks. Data are represented as mean±S.D.*≤0.05, **≤0.01.

FIG. 9D: Fat and lean mass of control (n=6), CRISPRi targeting promoter (n=6), or enhancer (n=6) of Adgrg6 measured by dual energy X-ray absorptiometry (DEXA).

FIG. 9E: Tissue weight of control (n=3), CRISPRi targeting promoter (n=3), or enhancer (n=3) of Adgrg6 mice. Data are represented as mean±S.D. *≤0.05, ***≤0.001.

FIG. 9F: Glucose tolerance test (GTT) and insulin tolerance test (ITT) of control (n=6), CRISPRi targeting promoter (n=6), or enhancer (n=6) of Adgrg6. Data are represented as mean±S.D*≤0.05 **≤0.01, ***≤0.001.

FIG. 10A: qRT-PCR of Adgrg6 in mouse 3T3-L1 preadipocytes transfected with dCas9 and various gRNAs targeting the promoter or the enhancer of Adgrg6. Data are represented as mean±S.D. ***≤0.001.

FIG. 10B: qRT-PCR of Adgrg6 in mouse 3T3-L1 preadipocytes transduced with AAV9-dCas9 and 2 gRNAs targeting the promoter or 1 gRNA targeting the enhancer of Adgrg6. Data are represented as mean±S.D. **≤0.01.

FIG. 10C: qRT-PCR of ADGRG6 in human preadipocytes transfected with dCas9 and various gRNAs targeting the promoter or the enhancer of ADGRG6. Data are represented as mean±S.D. *≤0.05, **≤0.01.

### DETAILED DESCRIPTION OF THE DISCLOSURE

### I. Introduction

The present disclosure is directed to modifying the Adgrg6 gene to reduce body fat in a male subject. The inventors have found that the Adgrg6 gene is associated with gender-specific fat distribution and mutating or reducing the expression of the Adgrg6 gene can reduce body fat in the male subject, leading to reduced risk in developing metabolic diseases, such as such as type 2 diabetes and cardiovascular diseases.

ADGRG6 is a G-protein coupled receptor that is involved in the formation of the myelin sheath, regulates Schwann cell differentiation via activation of cyclin adenosine monophosphate (cAMP) [40-42], and maintains connective tissue in intervertebral disc [43, 44], inner ear [45], ventricles [46], and placenta [47]. Ablation of Adgrg6 in mouse 3T3-L1 adipocytes has been shown to prevent adipocyte differentiation [48]. The ADGRG6 locus is also associated with adolescent idiopathic scoliosis (AIS), and several enhancers in this locus were previously characterized due to this association [49]. However, there is very little known about the role of ADGRG6 in adipose tissue.

A noncoding single nucleotide polymorphism (SNP), rs6570507, near the adhesion G protein-coupled receptor G6 (ADGRG6; also called GPR126) was found to be associated with female trunk fat in GWAS for gender-specific fat distribution [39]. The inventors found that SNP rs9403383 has high linkage disequilibrium (LD) with rs6570507 (r2=0.99), leading to reduced enhancer activity. Association analysis using the UK Biobank [50, 51] also showed that rs9403383 is highly associated with female trunk fat (p value=5.03478e-13), but not male trunk fat. Transcription factor binding site and chromatin immunoprecipitation analyses demonstrate that the associated SNP affects HoxA3, GR (glucocorticoid receptor), and PGR (progesterone receptor) binding to an enhancer region. Knockout of this gene or enhancer in human adipocytes leads to impaired adipogenesis.

Conditional knockout of Adgrg6 in adipocytes in mice using two different promoters to express Cre (Pdgfra and Fabp4) leads to fat deposition differences, making males more female like and showing improved glucose tolerance and insulin sensitivity. Furthermore, removal of the adipocyte enhancer in mice similarly leads to female-like fat deposition and lower body in male mice. Finally, the disclosure shows that CRISPRi targeting of the promoter or enhancer of Adgrg6 prevents high-fat-diet induced obesity and improves insulin response. Combined, the results identify ADGRG6 as an important adipogenesis factor regulating gender fat deposition and showcase its use as a therapeutic target to treat obesity and its co-associated morbidities.

### II. Definitions

Unless specifically indicated otherwise, all technical and scientific terms used herein have the same meaning as commonly understood by those of ordinary skill in the art to which this disclosure belongs. In addition, any method or material similar or equivalent to a method or material described herein can be used in the practice of the present disclosure. For purposes of the present disclosure, the following terms are defined.

As used herein, the term “metabolic disease” refers to a disease, disorder, or syndrome that is related to a subject's metabolism, such as breaking down carbohydrates, proteins, and fats in food to release energy, and converting chemicals into other substances and transporting them inside cells for energy utilization and/or storage. Some symptoms of a metabolic disease include high serum triglycerides, high low-density cholesterol (LDL), low high-density cholesterol (HDL), and/or high fasting insulin levels, elevated fasting plasma glucose, abdominal (central) obesity, and elevated blood pressure. Metabolic diseases increase the risk of developing other diseases, such as cardiovascular disease. Examples of metabolic diseases include, but are not limited to, obesity, Type-1 diabetes, and Type-2 diabetes.

As used herein, the term “adiposity” refers to the fat stored in the adipose tissue of a subject.

As used herein, the term “lean mass” refers to a component of body composition which includes, e.g., lean mass, body fat, and body fluid. Lean mass can be calculated by subtracting the weights of body fat and body fluid from total body weight. Typically, a subject's lean mass is between 60% and 90% of totally body weight.

As used herein, the term “rate of glucose clearance” refers to the rate at which glucose is being cleared from the blood. In some embodiments, the rate of glucose clearance can be measured in a glucose tolerance test (GTT). In a GTT, a subject is given a certain amount of glucose and blood samples are taken afterward to determine how quickly it is cleared from the blood. In other embodiments, the rate of glucose clearance can be measured in an insulin tolerance test (ITT). The rate of glucose clearance can be used as a parameter in diagnosing and/or determining the risk of developing metabolic diseases such as obesity, diabetes, and insulin resistance.

As used herein, the term “about,” when followed by a specific numeric value that does not indicate the number of days, e.g., about 70%, refers to a range of values that is ±20% of the specific value. For example, “about 70%” includes ±20% of 70%, or from 56% to 84%. When the specific value is a percentage, the upper limit is 100%. Thus, about 95% refers to from 75% to 100%. Such a range performs the desired function or achieves the desired result. For example, “about” may refer to an amount that is within less than 20% of, less than 10% of, within less than 5% of, within less than 1% of, within less than 0.1% of, and within less than 0.01% of the specific value.

As used herein, the term “subject,” “individual,” and “patient” are used interchangeably herein to refer to a vertebrate, preferably a mammal, more preferably a human. Mammals include, but are not limited to, mice, murines, rats, simians, humans, farm animals, sport animals, and pets.

### III. Compositions and Methods Involving Adgrg6 Gene

Described herein are compositions and methods for reducing body fat in a human male subject. The compositions and methods are related to mutating or reducing the expression of an Adgrg6 gene in one or more cells in the human male subject. The Adgrg6 gene (UniProt ID NO.: Q86SQ4) was found to be an important adipogenesis factor regulating gender specific fat deposition. The disclosure provides compositions and methods that use this gene as a therapeutic target to treat metabolic diseases, such as obesity and Type-2 diabetes.

As demonstrated herein, mutating or reducing the expression of the Adgrg6 gene led to reduced abdominal visceral adipose tissue (VAT) in the human male subject. The methods described herein can also reduce body weight of the human male subject without affecting the subject's lean body mass. In some embodiments, the human male subject has or is at risk for developing Type-2 diabetes and the methods described herein can reduce blood glucose by mutating or reducing the expression of the Adgrg6 gene. In some embodiments, the human male subject has or is at risk for developing Type-2 diabetes or has Type-1 diabetes and the methods described herein can increase insulin sensitivity in the subject by mutating or reducing the expression of the Adgrg6 gene.

In some embodiments, one or more cells of the human male subject can be isolated and undergo gene therapy ex vivo to mutate or reduce the expression of the Adgrg6 gene in the cells. Once ex vivo gene therapy is complete, the altered cells can be reintroduced into the human male subject. In certain embodiments, adipose stem cells or adipose progenitor cells can be isolated from the human male subject to undergo ex vivo gene therapy to mutate or reduce the expression of the Adgrg6 gene. The disclosure also provides an isolated adipose cell (e.g., an adipose stem cell or progenitor cell) having a mutated Adgrg6 gene or an Adgrg6 gene with reduced expression compared to a wild-type adipose cell. The isolated adipose cell can contain a catalytically-inactive nuclease and a gRNA that targets a portion of a promoter or enhancer sequence operably linked to a coding sequence of the Adgrg6 gene. The catalytically-inactive nuclease (e.g., a dCas9) can be linked to a transcriptional repressor domain (e.g. KRAB). In certain embodiments, the gRNA targets the promoter sequence comprising the sequence of SEQ ID NO:8 and can comprise a sequence having at least 90%, 95%, 98%, 99% or 100% identity to a sequence of any one of SEQ ID NOS: 3-7. In certain embodiments, the gRNA targets the enhancer sequence comprising the sequence of SEQ ID NO: 14 and can comprise a sequence having at least 90%, 95%, 98%, 99% or 100% identity to a sequence of any one of SEQ ID NOS: 9-13.

### Mutating Adgrg6 Gene

The disclosure provides methods for reducing body fat in a human male subject, in which the Adgrg6 gene is mutated in one or more cells of the human male subject. Mutating the Adgrg6 gene can include knocking out the gene or introducing a nucleotide deletion or insertion into the gene.

Methods to knock out the Adgrg6 gene can include introducing into the one or more cells of the human male subject a nuclease targeted to the Adgrg6 gene. A nuclease can be an endonuclease, zinc finger nuclease, TALEN, site-specific recombinase, transposase, topoisomerase, and includes modified derivatives and variants thereof. Descriptions of nucleases that can be used in methods of the disclosure are provided further herein. In some embodiments, the nuclease can be a RNA-guided nuclease, such as a clustered regularly interspaced short palindromic repeats (CRISPR) nuclease. Methods to knock out the Adgrg6 gene can include introducing into the one or more cells of the human male subject a CRISPR nuclease and a guide RNA (gRNA) that targets a portion of the Adgrg6 gene. In certain embodiments, the gRNA comprises a sequence having at least 90%, 95%, 98%, 99% or 100% identity to a sequence of SEQ ID NO:1 or 2.

Techniques for site-directed mutagenesis to introduce a nucleotide deletion or insertion into the Adgrg6 gene include, but are not limited to, e.g., polymerase chain reaction (PCR), primer extension, and inverse PCR. During PCR, the primers are designed to include the desired change, which could be base substitution, addition, or deletion. The mutation is incorporated into the amplicon, replacing the original sequence. Site-directed mutagenesis by primer extension involves incorporating mutagenic primers in independent, nested PCRs before combining them in the final product. The reaction requires flanking primers complementary to the ends of the target sequence, and two internal primers with complementary ends. These internal primers contain the desired mutation and will hybridize to the region to be altered. Inverse PCR enables amplification of a region of sequence using primers oriented in the reverse direction. Using primers incorporating the desired change, an entire circular plasmid is amplified to change the desired sequence. Other techniques for site-directed mutagenesis to introduce a nucleotide deletion or insertion into the Adgrg6 gene are described in, e.g., Aiyar et al., Methods Mol Biol. 1996;57:177-91, and Shimada, Methods Mol Biol. 1996;57:157-65.

### Reducing Adgrg6 Gene Expression

The disclosure further provides methods for reducing body fat in a human male subject, in which the expression of the Adgrg6 gene is reduced. Techniques to reduce the expression of the Adgrg6 gene can include, but are not limited to, e.g., CRISPR interference (CRISPRi), RNA interference (RNAi), and antisense therapy.

### CRISPR Interference (CRISPRi)

CRISPRi utilizes a catalytically-inactive nuclease containing one or more amino acid mutations relative to a wild-type CRISPR nuclease and sterically represses transcription by blocking either transcriptional initiation or elongation. CRISPRi further includes a gRNA targeting a promoter or enhancer sequence of the gene whose expression is to be reduced. In eukaryotes, CRISPRi can also repress transcription via an effector domain. The catalytically-inactive nuclease can be linked to a repressor domain to allow transcription to be further repressed. For example, the well-studied Krüppel associated box (KRAB) domain can be fused to the catalytically-inactive nuclease to repress transcription of the Adgrg6 gene. In certain embodiments, the catalytically-inactive nuclease is a dead Cas9 (dCas9). Other examples of catalytically-inactive nucleases are described in detail further herein.

In some embodiments, methods for reducing body fat in a human male subject can include introducing into the one or more cells of the human male subject a catalytically-inactive nuclease (e.g., a dCas9) linked to a transcriptional repressor domain (e.g., KRAB) and a gRNA that targets a portion of a promoter sequence operably linked to a coding sequence of the Adgrg6 gene. A promoter sequence operably linked to a coding sequence of the Adgrg6 gene can comprise the sequence of SEQ ID NO:8. In specific embodiments, the gRNA targeting the promoter sequence can comprise a sequence having at least 90%, 95%, 98%, 99% or 100% identity to a sequence of any one of SEQ ID NOS: 3-7.

Further, methods for reducing body fat in a human male subject can include introducing into the one or more cells of the human male subject a catalytically-inactive nuclease (e.g., a dCas9) linked to a transcriptional repressor domain (e.g., KRAB) and a gRNA that targets a portion of an enhancer sequence operably linked to a coding sequence of the Adgrg6 gene. A enhancer sequence operably linked to a coding sequence of the Adgrg6 gene can comprise the sequence of SEQ ID NO:14. In specific embodiments, the gRNA targeting the enhancer sequence can comprise a sequence having at least 90%, 95%, 98%, 99% or 100% identity to a sequence of any one of SEQ ID NOS: 9-13.

### RNA Interference (RNAi)

RNA interference (RNAi) is a biological process in which RNA molecules (i.e., inhibitory RNA polynucleotides) are involved in sequence-specific suppression of gene expression. An inhibitory RNA polynucleotide can be synthesized to target the Adgrg6 gene to lower its expression level. The inhibitory RNA polynucleotide can be of various lengths, e.g., between 15 and 30 nucleotides (e.g., 15, 16, 17, 18, 19, 20, 21, 22, 23, 24, 25, 26, 27, 28, 29, or 30 nucleotides). In further embodiments, the inhibitory RNA polynucleotide can be single-stranded or double-stranded. The inhibitory RNA polynucleotide can specifically hybridize to or is complementary (e.g., partially complementary) to a portion of the Adgrg6 gene, such that stable and specific binding occurs between the inhibitory RNA polynucleotide and the gene. There is a sufficient degree of complementarity between the inhibitory RNA polynucleotide and the Adgrg6 gene to avoid non-specific binding of the inhibitory RNA polynucleotide to non-target sequences.

The inhibitory RNA polynucleotides described herein can be a microRNA, which is a single-stranded RNA molecule of about 21-23 nucleotides (e.g., 21, 22, or 23 nucleotides) in length. miRNAs are encoded by genes from whose DNA they are transcribed, but miRNAs are not translated into protein (non-coding RNA); instead, each primary transcript (a pri-miRNA) is processed into a short stem-loop structure called a pre-miRNA and finally into a functional mature miRNA. Mature miRNA molecules are either partially or completely complementary to one or more messenger RNA (mRNA) molecules.

miRNAs are first transcribed as primary transcripts or pri-miRNA with a cap and poly-A tail and processed to short, nucleotide stem-loop structures known as pre-miRNA in the cell nucleus by a protein complex known as the Microprocessor complex, consisting of the nuclease Drosha and the double-stranded RNA binding protein Pasha (Denli et al., Nature, 432:231-235,2004). These pre-miRNAs are then processed to mature miRNAs in the cytoplasm by interaction with the endonuclease Dicer, which also initiates the formation of the RNA-induced silencing complex (RISC) (Bernstein et al., Nature, 409:363-366, 2001). Either the sense strand or antisense strand of DNA can function as templates to give rise to miRNA. When Dicer cleaves the pre-miRNA stem-loop, two complementary short RNA molecules are formed, but only one is integrated into the RISC complex. This strand is known as the guide strand and is selected by the argonaute protein, which is the catalytically active RNase in the RISC complex, on the basis of the stability of the 5′ end (Preall et al., Curr. Biol., 16:530-535, 2006). The remaining strand, known as the anti-guide or passenger strand, is degraded as a RISC complex substrate). After integration into the active RISC complex, miRNAs base pair with their complementary mRNA molecules and induce target mRNA degradation and/or translational silencing.

Mammalian miRNA molecules are usually complementary to a site in the 3′ UTR of the target mRNA sequence (e.g., a portion of the Adgrg6 mRNA). In some embodiments, the annealing of the miRNA to the target mRNA inhibits protein translation by blocking the protein translation machinery. In some embodiments, the annealing of the miRNA to the target mRNA facilitates the cleavage and degradation of the target mRNA.

The inhibitory RNA polynucleotides described herein can also be a small interfering RNA (siRNA), which refers to a double stranded RNA with the two complementary strands each having between 15 and 20 nucleotides (e.g., 15, 16, 17, 18, 19, or 20 nucleotides). In some embodiments, the two strands of an siRNA molecule can each have a 3′-end overhang of two or three nucleotides. In an siRNA molecule, one strand (e.g., the antisense strand) is guiding and complementary (e.g., partially complementary) to the target gene (e.g., the Adgrg6 gene).

Suitable siRNA sequences can be identified using methods known in the art. For example, prediction algorithms that predict potential siRNA-targets based upon complementary DNA sequences in the target genes are available in the art. TargetScanHuman, for example, is a comprehensive web resource for inhibitory RNA-target predictions, and uses an algorithm that incorporates current biological knowledge of inhibitory RNA-target rules including seed-match model, evolutionary conservation, and free binding energy (Li and Zhang, Wiley Interdiscip Rev RNA 6:435-452, 2015 and Agarwal et al., Elife 4, 2015). In some embodiments, to further enhance silencing efficiency of the siRNA sequences, potential siRNA sequences may be analyzed to identify sites that do not contain regions of homology to other coding sequences, e.g., in the target cell or organism.

Once a potential siRNA sequence has been identified, a complementary sequence (i.e., an antisense strand sequence) can be designed. A potential siRNA sequence can also be analyzed using a variety of criteria known in the art. For example. to enhance their silencing efficiency, the siRNA sequences may be analyzed by a rational design algorithm to identify sequences that have one or more of the following features: (1) G/C content of about 25% to about 60% G/C; (2) at least 3 A/Us at positions 15-19 of the sense strand; (3) no internal repeats; (4) an A at position 19 of the sense strand; (5) an A at position 3 of the sense strand; (6) a U at position 10 of the sense strand; (7) no G/C at position 19 of the sense strand; and (8) no G at position 13 of the sense strand. The siRNA design tools that incorporate algorithms that assign suitable values of each of these features and are useful for selection of the siRNA are available in the art. One of skill in the art will appreciate that sequences with one or more of the foregoing characteristics may be selected for farther analysis and testing as potential siRNA sequences.

The inhibitory RNA polynucleotides described herein can also be a small hairpin RNA or short hairpin RNA (shRNA), which is a short RNA sequence that makes a tight hairpin turn that can be used to silence gene expression via RNA interference. The shRNA hairpin structure is cleaved by the cellular machinery into siRNA, which is then bound to the RNA-induced silencing complex (RISC). In some embodiments, shRNAs can be between 15 to 60 nucleotides (e.g., 15, 20, 25, 30, 35, 40, 45, 50, 55, or 60 nucleotides) in length. Non-limiting examples of shRNA include a double-stranded polynucleotide molecule assembled from a single-stranded molecule, in which the sense and antisense regions are linked by a nucleic acid-based or non-nucleic acid-based linker; and a double-stranded polynucleotide molecule with a hairpin secondary structure having self-complementary sense and antisense regions.

### Antisense Therapy

Antisense therapy is a gene expression suppression technique that uses antisense oligonucleotides (ASOs) to target mRNAs (e.g., Adgrg6 mRNA). Antisense therapy uses a naturally occurring enzyme, RNase H, that has the activity of destroying the RNA strand of an RNA:DNA duplex. In antisense therapy, a DNA oligonucleotide of approximately 15-30 bases in length that was complimentary to the target gene is introduced into the cells. Once inside the cell, the oligonucleotide base pairs with its targeted region in the target gene and when RNase H binds to the resulting duplex, it cleaves the RNA in multiple places, leaving the DNA oligonucleotide intact to help catalyze destruction of more target mRNAs. In this way, the production of the protein encoded by the targeted mRNA is greatly reduced.

### Small MOLECULES Targeting G-Protein Coupled Receptors

Since the ADGRG6 protein (encoded by the ADGRG6 gene) is a G-protein coupled receptor (GPCR), small molecules that target GPCRs, such as GPCR antagonists, can also be used in compositions and methods of the disclosure. Examples of small molecules that target ADGRG6 protein include, but are noted limited to, valproic acid, 4-(5-benzo(1,3)dioxol-5-yl-4-pyridin-2-yl-1H-imidazol-2-yl)benzamide, dorsomorphin, tetrachlorodibenzodioxin, acetaminophen, benzo(a)pyrene, bisphenol A, estradiol, tretinoin, and trichostatin A. Other examples of small molecules that target GPCR include, but are noted limited to, alfuzosin, terazosin, clonidine, bisoprolol, betaxolol, metoprolol, atenolol, albuterol, nadolol, penbutolol, tolterodine, atropine, scopolamine, calcimar, metoclopramide, haloperidol, olanzapine, ropinirole, pramipexole, loratadine, cetirizine, demenhydrinate, cimetidine, ranitidine, trazodone, sumatriptan, exenatide, fentanyl, codein, meperidine, oxycodone, montelukast, misoprostol, clopidogrel, aripiprazole, quetiapine, montelukast, olanzapine, and valsartan.

SNP rs9403383

The disclosure also provides methods that use the single nucleotide polymorphism (SNP) rs9403383 to reduce body fat in a human male subject. The inventors have found that SNP rs9403383 is strongly associated with female trunk fat but not male trunk fat. Further, as demonstrated in the Examples, rs9403383 significantly reduced the activity of the enhancer ADGRG6_4 associated with the Adgrg6 gene. Moreover, a human preadipocyte cell line containing the SNP rs9403383 showed strong reduction in Adgrg6 mRNA level.

As described herein, methods for reducing body fat in a human male subject can include reducing the expression of the Adgrg6 gene by knocking in the SNP rs9403383 at a location that is proximal to the Adgrg6 gene in one or more cells of the human male subject. In one example, SNP rs9403383 can be knocked in at chr6: 142-372-912 (GRCh38.p13).

In some embodiments, an RNA-guided nuclease (e.g., CRISPR nuclease), a gRNA, and a homology-directed-repair template (HDRT) comprising the SNP rs9403383 can be introduced into one or more cells of the human male subject to knock in the SNP. In certain embodiments, the gRNA comprises a sequence having at least 90%, 95%, 98%, 99% or 100% identity to a sequence of SEQ ID NO:15. In certain embodiments, the HDRT comprising the SNP rs9403383 has a sequence having at least 90%, 95%, 98%, 99% or 100% identity to a sequence of SEQ ID NO: 16.

### Subject

As described herein, the inventors have found the Adgrg6 gene to be involved in gender-specific fat distribution and adipocyte differentiation and that mutating (e.g., knocking out) or reducing the expression of the Adgrg6 gene can reduce body fat in a human male subject and further lower the risk of the human male subject developing a metabolic disease. In some embodiments, the metabolic disease can be obesity, Type-1 diabetes, Type-2 diabetes, or a cardiovascular disease. In some embodiments, the human male subject has or is at risk for developing obesity, i.e., has a body mass index (BMI) that is greater than 30 kg/m². In certain embodiments, the obesity is diet-induced obesity. In certain embodiments, the human male subject is overweight, i.e., has a body mass index (BMI) that is greater than 25 kg/m².

In some cases, a human male subject may desire to alter his body fat distribution. In certain embodiments, the human male subject may be undergoing a sex reassignment therapy to change into a female subject. Thus, by mutating (e.g., knocking out) or reducing the expression of the Adgrg6 gene, the human male subject can achieve a body fat distribution that is similar to female body fat distribution.

### IV. Methods of Delivery

In compositions and methods described herein, the RNA-guided nuclease and its associated components (e.g., gRNA, HDRT) can be delivered into one or more cells (e.g., adipocytes, adipose stem cells or adipose progenitor cells) of the human male subject using a number of techniques in the art. In some embodiments, the composition can be introduced into the cell via electroporation. In some embodiments, a ribonucleoprotein (RNP) complex containing a Cas protein (e.g., Cas9 nuclease) and a gRNA can be formed first, then electroporated into the cell. Methods, compositions, and devices for electroporation are available in the art, e.g., those described in WO2006/001614 or Kim, J. A. et al. Biosens. Bioelectron. 23, 1353-1360 (2008). Additional or alternative methods, compositions, and devices for electroporation can include those described in U.S. Patent Appl. Pub. Nos. 2006/0094095; 2005/0064596; or 2006/0087522. Additional or alternative methods, compositions, and devices for electroporation can include those described in Li, L. H. et al. Cancer Res. Treat. 1, 341-350 (2002); U.S. Pat. Nos.: 6,773,669; 7,186,559; 7,771,984; 7,991,559; 6,485,961; and 7,029,916; and U.S. Patent Appl. Pub. Nos: 2014/0017213; and 2012/0088842. Additional or alternative methods, compositions, and devices for electroporation can include those described in Geng, T. et al. J. Control Release 144, 91-100 (2010); and Wang, J., et al. Lab Chip 10, 2057-2061 (2010).

In some embodiments, the Cas protein, the gRNA, and the HDRT can be introduced into the cell via viral delivery using a viral vector. For example, viral vectors can be based on vaccinia virus, poliovirus, adenovirus, adeno-associated virus (AAV) (e.g., recombinant AAV (rAAV)), SV40, herpes simplex virus, human immunodeficiency virus, and the like. A retroviral vector can be based on Murine Leukemia Virus, spleen necrosis virus, and vectors derived from retroviruses such as Rous Sarcoma Virus, Harvey Sarcoma Virus, avian leukosis virus, a lentivirus (e.g., integration deficient lentivirus), human immunodeficiency virus, myeloproliferative sarcoma virus, mammary tumor virus, and the like. In some embodiments, a retroviral vector can be an integration deficient gamma retroviral vector. Other useful expression vectors are known to those of skill in the art, and many are commercially available. The following exemplary vectors are provided by way of example for eukaryotic host cells: pXT1, pSG5, pSVK3, pBPV, pMSG, and pSVLSV40. Examples of techniques that may be used to introduce a viral vector into a cell include, but not limited to, viral or bacteriophage infection, transfection, protoplast fusion, lipofection, calcium phosphate precipitation, polyethyleneimine (PEI)-mediated transfection, DEAE-dextran mediated transfection, liposome-mediated transfection, calcium phosphate precipitation, nanoparticle-mediated nucleic acid delivery, and the like.

Further, isolated cells (e.g., adipocytes, adipose stem cells, adipose progenitor cells) can undergo ex vivo gene therapy to mutate or reduce the expression of the Adgrg6 gene, and the modified cells can be introduced into the human male subject. Ex vivo gene therapy is a therapeutic approach that typically includes isolation and ex vivo expansion and/or manipulation of cells and subsequent administration of these cells to a patient. The isolated cells may be manipulated to mutate or reduce the expression of the Adgrg6 gene in any one of the known ways, including, for example, by using RNA and DNA transfection, viral transduction, electroporation, all of which are technologies known in the art. In some embodiments, the isolated cells can be from the same human male subject (i.e., autologous cells). In other embodiments, the isolated cells can be from a different subject (i.e., allogenic cells).

### VI. Nucleases

As described herein, a nuclease can be used in methods and compositions of the disclosure to mutate or reduce the expression of the Adgrg6 gene. Examples of nucleases include, but are not limited to, an endonuclease, zinc finger nuclease, TALEN, site-specific recombinase, transposase, topoisomerase, and modified derivatives and variants thereof.

In some embodiments, a nuclease is capable of targeting a designated nucleotide or region within the target gene (e.g., Adgrg6 gene). In some embodiments, the nuclease is capable of targeting a region positioned between the 5′ and 3′ regions of the target gene. In another embodiment, the nuclease is capable of targeting a region positioned upstream or downstream of the 5′ and 3′ regions of the target gene (e.g., upstream or downstream of the transcription start site (TSS)). A recognition sequence is a polynucleotide sequence that is specifically recognized and/or bound by the nuclease. The length of the recognition site sequence can vary, and includes, for example, nucleotide sequences that are at least 10, 12, 14, 16, 18, 19, 20, 21, 22, 23, 24, 25, 26, 27, 28, 29, 30, 35, 40, 45, 50, 55, 60, 65, 70 or more nucleotides in length.

### Cas Endonuclease

In some embodiments, the nuclease used in methods and compositions of the disclosure is a CRISPR-associated (Cas) protein. A Cas protein refers to an RNA-guided double-stranded DNA-binding nuclease protein or nickase protein. In methods and compositions described herein, a Cas protein can be used to knock out the Adgrg6 gene by cleaving the Adgrg6 gene or a portion thereof. Wild-type Cas nuclease has two functional domains, e.g., RuvC and HNH, that cut different DNA strands. A Cas protein can induce double-strand breaks in genomic DNA (target nucleic acid) when both functional domains are active. The Cas protein can comprise one or more catalytic domains of a Cas protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas protein can be a fusion protein, e.g., the two catalytic domains are derived from different bacteria species.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, variants thereof, mutants thereof, and derivatives thereof. There are three main types of Cas proteins (type I, type II, and type III), and 10 subtypes including 5 type I, 3 type II, and 2 type III proteins (see, e.g., Hochstrasser and Doudna, Trends Biochem Sci, 2015: 40(1):58-66). Type II Cas proteins include Cas1, Cas2, Csn2, Cas9, and Cfp1. These Cas proteins are known to those skilled in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI Ref. Seq. No. WP_011681470.

Cas proteins, e.g., Cas9 nucleases, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

In some embodiments, a Cas protein can be a Cas protein variant. For example, useful variants of the Cas9 nuclease can include a single inactive catalytic domain, such as a RuvC⁻ or HNH⁻ enzyme or a nickase. A Cas9 nickase has only one active functional domain and can cut only one strand of the target nucleic acid, thereby creating a single strand break or nick. In some embodiments, the Cas9 nuclease can be a mutant Cas9 nuclease having one or more amino acid mutations. For example, the mutant Cas9 having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. A double-strand break can be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

In some embodiments, a Cas protein variant that lacks cleavage (e.g., nickase) activity. A Cas protein variant may contain one or more point mutations that eliminates the protein's nickase activity. In some embodiments, Cas protein variants that lack cleavage activity can bind to a Cas protein target sequence via a gRNA that hybridizes to the Cas protein target sequence. In other embodiments, Cas protein variants that lack cleavage activity can be fused to other proteins and serve as targeting domains to direct the other proteins to the target nucleic acid. For example, Cas protein variants without nickase activity may be fused to transcriptional activation or repression domains to control gene expression (Ma et al., Protein and Cell, 2 (11):879-888, 2011; Maeder et al., Nature Methods, 10:977-979, 2013; and Konermann et al., Nature, 517:583-588, 2014).

In some embodiments, a Cas protein variant without any cleavage activity can be a Cas9 polypeptide that contains two silencing mutations of the RuvC1 and HNH nuclease domains (D10A and H840A), which is referred to as dCas9 (Jinek et al., Science, 2012, 337:816-821; Qi et al., Cell, 152(5):1173-1183). In one embodiment, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Publication No. WO 2013/176772. The dCas9 enzyme can contain a mutation at D10, E762, H983, or D986, as well as a mutation at H840 or N863. In some instances, the dCas9 enzyme can contain a D10A or D10N mutation. Also, the dCas9 enzyme can contain a H840A, H840Y, or H840N. In some embodiments, the dCas9 enzyme can contain D10A and H840A; D10A and H840Y; D10A and H840N; DION and H840A; DION and H840Y; or D10N and H840N substitutions. The substitutions can be conservative or non-conservative substitutions to render the Cas9 polypeptide catalytically inactive and able to bind to target nucleic acid. In methods and compositions described herein, a catalytically-inactive Cas protein (e.g., dCas9), optionally linked to a repressor domain (e.g., KRAB), can be used to target a promoter or enhancer sequence of the Adgrg6 gene in order to reduce the expression of the Adgrg6 gene.

In some embodiments, the Cas protein can be a high-fidelity or enhanced specificity Cas9 polypeptide variant with reduced off-target effects and robust on-target cleavage. Non-limiting examples of Cas9 polypeptide variants with improved on-target specificity include the SpCas9 (K855A), SpCas9 (K810A/K1003A/R1060A) (also referred to as eSpCas9(1.0)), and SpCas9 (K848A/K1003A/R1060A) (also referred to as eSpCas9(1.1)) variants described in Slaymaker et al., Science, 351(6268):84-8 (2016), and the SpCas9 variants described in Kleinstiver et al., Nature, 529(7587):490-5 (2016) containing one, two, three, or four of the following mutations: N497A, R661A, Q695A, and Q926A (e.g., SpCas9-HF1 contains all four mutations).

### Zinc Finger Nuclease

In some embodiments, the nuclease is a zinc-finger nuclease (ZFN). ZFNs typically comprise a zinc finger DNA binding domain and a nuclease domain. Generally, ZFNs include two zinc finger arrays (ZFAs), each of which is fused to a single subunit of a non-specific endonuclease, such as the nuclease domain from the FokI enzyme, which becomes active upon dimerization. Typically, a single ZFA consists of 3 or 4 zinc finger domains, each of which is designed to recognize a specific nucleotide triplet (GGC, GAT, etc.). A ZFN composed of two “3-finger” ZF As is therefore capable of recognizing an 18 base pair target site (i.e., recognition sequence); an 18 base pair recognition sequence is generally unique, even within large genomes such as those of humans and plants. By directing the co-localization and dimerization of the two FokI nuclease monomers, ZFNs generate a functional site-specific endonuclease that can target a particular locus (e.g., gene, promotor or enhancer).

Zinc-finger nucleases useful in the methods disclosed herein include those that are known and ZFN that are engineered to have specificity for one or more target sites described herein (e.g., promotor or enhancer nucleotide sequence). Zinc finger domains are amenable for designing polypeptides which specifically bind a selected polynucleotide recognition sequence within a target site of the host cell genome. ZFN can comprise an engineered DNA-binding zinc finger domain linked to a non-specific endonuclease domain, for example, a nuclease domain from a Type IIs endonuclease such as HO or FokI. In some examples, a zinc finger DNA binding domain can be fused to a site-specific recombinase, transposase, or a derivative thereof that retains DNA nicking and/or cleaving activity.

In some embodiments, additional functionalities can be fused to the zinc-finger binding domain, including but not limited to, transcriptional activator domains (such as VP16, VP48, VP64, VP160 and the like) or transcription repressor domains (such as KRAB). In one embodiment, the zinc finger nuclease is engineered such that the zinc finger nuclease comprises a transcriptional activator domain selected from VP16, VP48, VP64 or VP160. In one embodiment, the zinc finger nuclease is engineered such that the zinc finger nuclease comprises a transcriptional activator domain selected from HSF1, VP16, VP64, p65, RTA, MyoD1, SET7, VPR, histone acetyltransferase p300, TET1 hydroxylase catalytic domain, LSD1, CIB1, AD2, CR3, GATA4, p53, SP1, MEF2C, TAX, PPAR-gamma, and SET9. For example, engineered zinc finger transcriptional activator that interact with a promoter region of the gamma-globulin gene was shown to enhance fetal hemoglobin production in primer adult erythroblasts (Wilber et al., Blood, 115(15):3033-3041). Other polydactyl zinc-finger transcription factors are also known in the art, including those disclosed in Beerli and Barbas (see, Nature Technology, (2002) 20:135-141).

Each zinc finger domain recognizes three consecutive base pairs in the target DNA. For example, a three finger domain recognizes a sequence of nine contiguous nucleotides, with a dimerization requirement of the nuclease, two sets of zinc finger triplets are used to bind a 18 nucleotide recognition sequence. Useful zinc finger modules include those that recognize various GNN and ANN triplets (Dreier et al., (2001) J Biol Chem 276:29466-78; Dreier et al., (2000) J Mol Biol 303:489-502; Liu et al., (2002) J Biol Chem 277:3850-6), as well as those that recognize various CNN or TNN triplets (Dreier et al., (2005) J Biol Chem 280:35588-97; Jamieson et al., (2003) Nature Rev Drug Discovery 2:361-8). See also, Durai et al., (2005) Nucleic Acids Res 33:5978-90; Segal, (2002) Methods 26:76-83; Porteus and Carroll, (2005) Nat Biotechnology 23:967-73; Pabo et al., (2001) Ann Rev Biochem 70:313-40; Wolfe et al., (2000) Ann Rev Biophys Biomol Struct 29:183-212; Segal and Barbas (2001) Curr Opin Biotechnol 12:632-7; Segal et al., (2003) Biochemistry 42:2137-48; Beerli and Barbas, (2002) Nat Biotechnol 20:135-41; Carroll et al., (2006) Nature Protocols 1:1329; Ordiz et al., (2002) Proc Natl Acad Sci USA 99:13290-5; Guan et al., (2002) Proc Natl Acad Sci USA 99:13296-301;WO2002099084; WO00/42219; WO02/42459; WO2003062455; US20030059767; US Patent Application Publication Number 2003/0108880; U.S. Pat. Nos. 6,140,466, 6,511,808 and 6,453,242. Useful zinc-finger nucleases also include those described in WO03/080809; WO05/014791; WO05/084190; WO08/021207; WO09/042186; WO09/054985; and WO10/065123.

In some embodiments, a ZFN comprises a fusion protein having a cleavage domain of a Type IIS restriction endonuclease fused to an engineered zinc finger binding domain, wherein the binding domain further comprises one or more transcriptional activators. In some embodiments, the type IIS restriction endonuclease is selected from a HO endonuclease or a FokI endonuclease. In some embodiments, the zinc finger binding domain comprises 3, 4, 5 or 6 zinc fingers. In another embodiment, the zinc finger binding domain specifically binds to a recognition sequence corresponding to a promoter or enhancer disclosed herein (e.g., SIM1, MC4R, PKD1, SETD5, THUMPD3, SCN2A and PAX6 promotor or enhancer). In one embodiment, the one or more transcriptional activators is selected from VP16, VP48, VP64, or VP160. Generally, the DNA-binding domain of a ZFN contains between 3 and 6 individual zinc finger repeats and can recognize between 9 and 18 contiguous nucleotides. Each ZFN can be designed to target a specific target site in the host cell genome, e.g., a promotor sequence, an enhancer sequence, or exon/intron within a gene.

### TALENS

In some embodiments of the methods and compositions described herein, the nuclease is a TALEN. TAL effectors (TALEs) are proteins secreted by Xanthomonas bacteria and play an important role in disease or triggering defense mechanisms, by binding host DNA and activating effector-specific host genes. see, e.g., Gu et al. (2005) Nature 435:1122-5; Yang et al., (2006) Proc. Natl. Acad. Sci. USA 103:10503-8; Kay et al., (2007) Science 318:648-51; Sugio et al., (2007) Proc. Natl. Acad. Sci. USA 104:10720-5; Romer et al., (2007) Science 318:645-8; Boch et al., (2009) Science 326(5959):1509-12; and Moscou and Bogdanove, (2009) 326(5959):1501. A TALEN comprises a TAL effector DNA-binding domain fused to a DNA cleavage domain. The DNA binding domain interacts with DNA in a sequence-specific manner through one or more tandem repeat domains. The repeated sequence typically comprises 33-34 highly conserved amino acids with divergent 12^{th} and 13^{th} amino acids. These two positions, referred to as the Repeat Variable Diresidue (RVD) are highly variable and show a strong correlation with specific nucleotide recognition (Boch et al., (2009) Science 326(5959):1509-12; and Moscou and Bogdanove, (2009) 326(5959):1501). This relationship between amino acid sequence and DNA recognition sequence has allowed for the engineering of specific DNA-binding domains by selecting a combination of repeat segments containing the appropriate RVDs.

The TAL-effector DNA binding domain can be engineered to bind to a target DNA sequence and fused to a nuclease domain, e.g., a Type IIS restriction endonuclease, such as FokI (see e.g., Kim et al. (1996) Proc. Natl. Acad. Sci. USA 93:1156-1160). In some embodiments, the nuclease domain can comprises one or more mutations (e.g., FokI variants) that improve cleavage specificity (see, Doyon et al., (2011) Nature Methods, 8 (1): 74-9) and cleavage activity (Guo et al., (2010) Journal of Molecular Biology, 400 (1): 96-107). Other useful endonucleases that can be used as the nuclease domain include, but are not limited to, HhaI, HindIII, Nod, BbvCI, EcoRI, Bg1I, and A1wI. In some embodiments, the TALEN can comprise a TAL effector DNA binding domain comprising a plurality of TAL effector repeat sequences that bind to a specific nucleotide sequence (i.e., recognition sequence) in the target DNA. While not to be construed as limiting, TALENs useful for the methods provided herein include those described in WO10/079430 and U.S. Patent Application Publication No. 2011/0145940.

In some embodiments, the TAL effector DNA binding domain can comprise 10 or more DNA binding repeats, and preferably 15 or more DNA binding repeats. In some embodiments, each DNA binding repeat comprises a RVD that determines recognition of a base pair in the target DNA, and wherein each DNA binding repeat is responsible for recognizing one base pair in the target DNA. In some embodiments, the RVD comprises one or more of: HD for recognizing C; NG for recognizing T; NI for recognizing A; NN for recognizing G or A; NS for recognizing A or C or G or T; N* for recognizing C or T, where * represents a gap in the second position of the RVD; HG for recognizing T; H* for recognizing T, where * represents a gap in the second position of the RVD; IG for recognizing T; NK for recognizing G; HA for recognizing C; ND for recognizing C; HI for recognizing C; HN for recognizing G; NA for recognizing G; SN for recognizing G or A; and YG for recognizing T.

In some embodiments, the TALEN is engineered such that the TAL effector comprises one or more transcriptional activator domains (e.g., VP16, VP48, VP64 or VP160). For example, engineered TAL effectors having a transcriptional activator domain at the c-terminus of the TAL effector were shown to modulate transcription of Sox2 and Klf4 genes in human 293 FT cells (Zhang et al., Nature Biotechnology, 29(2):149-153(2011). Other TAL effector transcription factors (TALE-TFs) are also known in the art, including those disclosed in Perez-Pinera et al., (Nature Methods, (2013) 10(3):239-242) that demonstrated modulation of IL1RN, KLK3, CEACAM5 and ERBB2 genes in human 293T cells using TALE-TFs. In some embodiments, the one or more transcriptional activator domains are located adjacent to the nuclear localization signal (NLS) present in the C-terminus of the TAL effector. In another embodiment, the TALE-TFs can bind nearby sites upstream or downstream of the transcriptional start site (TSS) for a target gene. In one embodiment, the TAL effector comprises a transcriptional activator domain selected from VP16, VP48, VP64 or VP160. In another embodiment, the TAL effector comprises a transcriptional activator domain selected from HSF1, VP16, VP64, p65, RTA, MyoD1, SET7, VPR, histone acetyltransferase p300, TET1 hydroxylase catalytic domain, LSD1, CIB1, AD2, CR3, GATA4, p53, SP1, MEF2C, TAX, PPAR-gamma, and SET9.

In some embodiments, the TALEN comprises a TAL effector DNA-binding domain fused to a DNA cleavage domain, wherein the TAL effector comprises a transcriptional activator. In some embodiments, the DNA cleavage domain is of a Type IIS restriction endonuclease selected from a HO endonuclease or a FokI endonuclease. In some embodiments, the TAL effector DNA-binding domain specifically binds to a recognition sequence corresponding to a promoter region or enhancer region disclosed herein (e.g., SIM1, MC4R, PKD1, SETD5, THUMPD3, SCN2A and PAX6 promotor or enhancer). Generally, the DNA-binding domain of a TALEN is designed to target a specific target site in the host cell, e.g., a promotor sequence or an enhancer sequence.

### V. Guide RNAs

A Cas protein can be guided to its target nucleic acid by a guide RNA (gRNA). A gRNA is a version of the naturally occurring two-piece guide RNA (crRNA and tracrRNA) engineered into a two-piece gRNA or a single, continuous sequence. A gRNA can contain a guide sequence (e.g., the crRNA equivalent portion of the gRNA) that targets the Cas protein to the target nucleic acid and a scaffold sequence that interacts with the Cas protein (e.g., the tracrRNAs equivalent portion of the gRNA). A gRNA can be selected using a software. As a non-limiting example, considerations for selecting a gRNA can include, e.g., the PAM sequence for the Cas protein to be used, and strategies for minimizing off-target modifications. Tools, such as NUPACK® and the CRISPR Design Tool, can provide sequences for preparing the gRNA, for assessing target modification efficiency, and/or assessing cleavage at off-target sites.

### Guide Sequence

The guide sequence in the gRNA may be complementary to a specific sequence within a target nucleic acid (e.g., the Adgrg6 gene). The 3′ end of the target nucleic acid sequence can be followed by a PAM sequence. Approximately 20 nucleotides upstream of the PAM sequence is the target nucleic acid. In general, a Cas9 protein or a variant thereof cleaves about three nucleotides upstream of the PAM sequence. The guide sequence in the gRNA can be complementary to either strand of the target nucleic acid.

In some embodiments, the guide sequence of a gRNA may comprise about 10 to about 2000 nucleic acids, for example, about 10 to about 100 nucleic acids, about 10 to about 500 nucleic acids, about 10 to about 1000 nucleic acids, about 10 to about 1500 nucleic acids, about 10 to about 2000 nucleic acids, about 50 to about 100 nucleic acids, about 50 to about 500 nucleic acids, about 50 to about 1000 nucleic acids, about 50 to about 1500 nucleic acids, about 50 to about 2000 nucleic acids, about 100 to about 500 nucleic acids, about 100 to about 1000 nucleic acids, about 100 to about 1500 nucleic acids, about 100 to about 2000 nucleic acids, about 500 to about 1000 nucleic acids, about 500 to about 1500 nucleic acids, about 500 to about 2000 nucleic acids, about 1000 to about 1500 nucleic acids, or about 1000 to about 2000 nucleic acids. In some embodiments, the guide sequence of a gRNA comprises about 100 nucleic acids at the 5′ end of the gRNA that can direct the Cas protein to the target nucleic acid site using RNA-DNA complementarity base pairing. In some embodiments, the guide sequence comprises about 20 nucleic acids at the 5′ end of the gRNA that can direct the Cas protein to the target nucleic acid site using RNA-DNA complementarity base pairing. In other embodiments, the guide sequence comprises less than 20, e.g., 19, 18, 17, 16, 15 or less, nucleic acids that are complementary to the target nucleic acid site. In some instances, the guide sequence in the gRNA contains at least one nucleic acid mismatch in the complementarity region of the target nucleic acid site. In some instances, the guide sequence contains about 1 to about 10 nucleic acid mismatches in the complementarity region of the target nucleic acid site.

### Scaffold Sequence

The scaffold sequence in the gRNA can serve as a protein-binding sequence that interacts with the Cas protein or a variant thereof. In some embodiments, the scaffold sequence in the gRNA can comprise two complementary stretches of nucleotides that hybridize to one another to form a double-stranded RNA duplex (dsRNA duplex). The scaffold sequence may have structures such as lower stem, bulge, upper stem, nexus, and/or hairpin. In some embodiments, the scaffold sequence in the gRNA can be between about 90 nucleic acids to about 120 nucleic acids, e.g., about 90 nucleic acids to about 115 nucleic acids, about 90 nucleic acids to about 110 nucleic acids, about 90 nucleic acids to about 105 nucleic acids, about 90 nucleic acids to about 100 nucleic acids, about 90 nucleic acids to about 95 nucleic acids, about 95 nucleic acids to about 120 nucleic acids, about 100 nucleic acids to about 120 nucleic acids, about 105 nucleic acids to about 120 nucleic acids, about 110 nucleic acids to about 120 nucleic acids, or about 115 nucleic acids to about 120 nucleic acids.

### EXAMPLES

The present disclosure will be described in greater detail by way of a specific example. The following example is offered for illustrative purposes, and is not intended to limit the disclosure in any manner. Those of skill in the art will readily recognize a variety of noncritical parameters which can be changed or modified to yield essentially the same results.

### Example 1. A Preadipocyte Enhancer Near ADGRG6 is Associated With Gender-Specific Fat Distribution

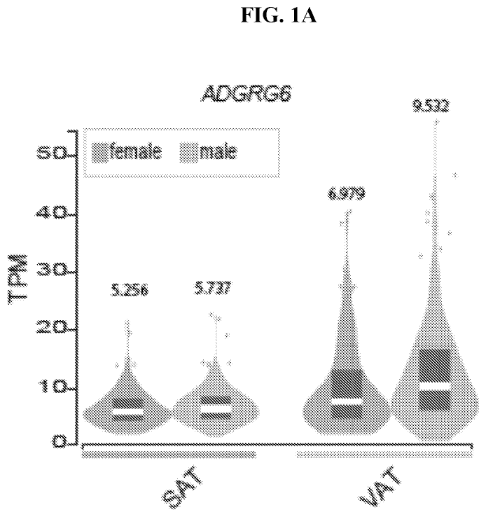

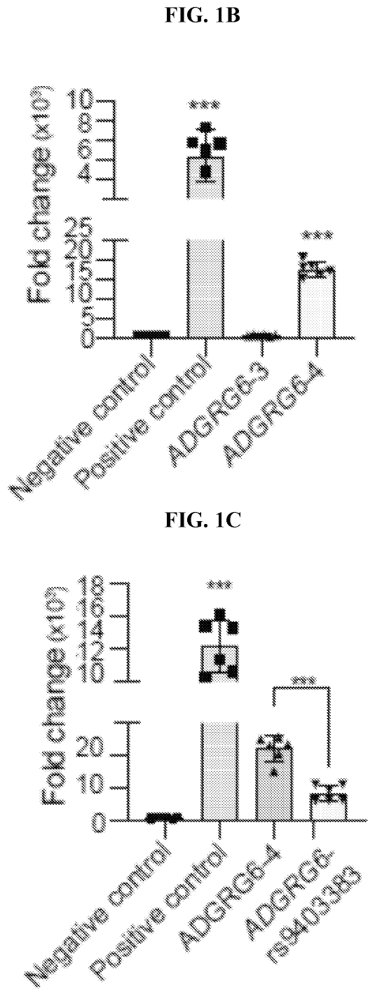

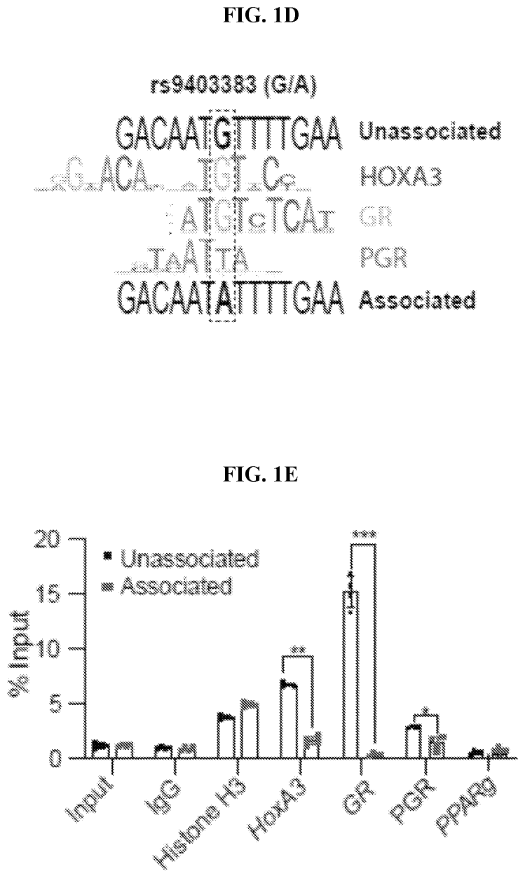

We identified a SNP near ADGRG6, rs6570507, to be associated with gender body fat distribution from segmental bioelectrical impedance analysis (sBIA) data [39]. rs6570507 was found to have a larger effect in female VAT than males. In addition, it is also associated with body fat distribution using waist-to-hip ratio adjusted for BMI and obesity in females [37, 52]. We examined ADGRG6 expression in the Genotype-Tissue Expression (GTEX) portal [53], finding higher expression in males versus females in VAT (FIG. 1A). rs9403383, which is in high LD with rs6570507 (r²=0.99), was found to be significantly associated with trunk fat mass (p-value=2.61×10⁻¹¹) in the UK Biobank [50, 51] and to have significant cis-eQTL association with both VAT (p-value=3.26×10⁻⁵) and SAT in the GTEX portal (p=5.2×10⁻¹³). To determine whether SNP rs9403383 at ADGRG6 was associated with trunk fat mass in humans and had different effects in men and women, we conducted sex-specific analyses in the European ancestry sample of the UK Biobank (UKB) cohort. While SNP rs9403383 was genome-wide significantly associated (p=5.03×10⁻¹³) with trunk fat mass in women (N=245,000), we observed no significant association (p=0.06) for this SNP in men (N=205,628).

We next set out to assess whether rs6570507 or SNPs in LD with it (r²>0.8) overlap potential active enhancer sequences, by analyzing H3K27ac ChIP-seq data from human adipose tissue [54]. We found two H3H27ac peaks, which were previously named in a previous study in our lab that characterize AIS-associated enhancers as ADGRG6_3 and ADGRG_4 [49], to overlap SNPs in LD with rs6570507 and rs9403383. To functionally validate whether sequences at both H3K27ac peaks might have adipocyte enhancer activity, we cloned both regions into an enhancer assay vector, pGL4.23 (Promega), that contains a minimal promoter followed by the luciferase reporter gene. We transfected these constructs into human preadipocytes, finding only ADGRG6_4 to have enhancer activity, showing ˜15-fold higher luciferase expression than the negative control (FIG. 1B). We next tested whether rs9403383 affects ADGRG6_4 enhancer activity, finding that the gender body fat distribution associated SNPs significantly reduced enhancer activity compared to the unassociated variant (FIG. 1C).

We next examined whether rs9403383 leads to any transcription factor binding site (TFBS) changes. Using PROMO and TRANSFAC [56, 57], we found that the unassociated sequence contains TFBS for multiple TFs that are critical for adipocyte differentiation, such as CCAAT/enhancer-binding protein delta (C/EBPd) and glucocorticoid receptor (GR). The associated variant could disrupt the TFBS of Homeobox protein A3 (HoxA3), progesterone receptor (PGR) and GR (FIG. 1D). We generated a human preadipocytes cell line carrying the rs9403383 associated variant by transfecting Cas9, gRNA, and a ssDNA donor sequence containing the associated variant. We selected for single cell colonies and validated them for the existence of this variant by sequencing. We then preformed ChIP-qPCR with either HOXA3, GR, or PGR antibodies on human preadipocytes containing either the unassociated or associated rs9403383 variant. We also used IgG and PPARg as negative controls and histone H3 as a positive control. We observed significantly decreased enrichment for all three transcription factors (HOXA3, GR, or PGR) in the cells having the associated allele (FIG. 1E), indicating that the associated rs9403383 variant affects the binding of these transcription factors to the enhancer sequence. Taken together, we identified a novel adipocyte enhancer in the intron of ADGRG6 that carries a SNP associated with gender-specific fat distribution and can hinder its activity.

### Example 2. ADGRG6 is Highly Expressed in Adipose Progenitors and Mesenchymal Stem Cells

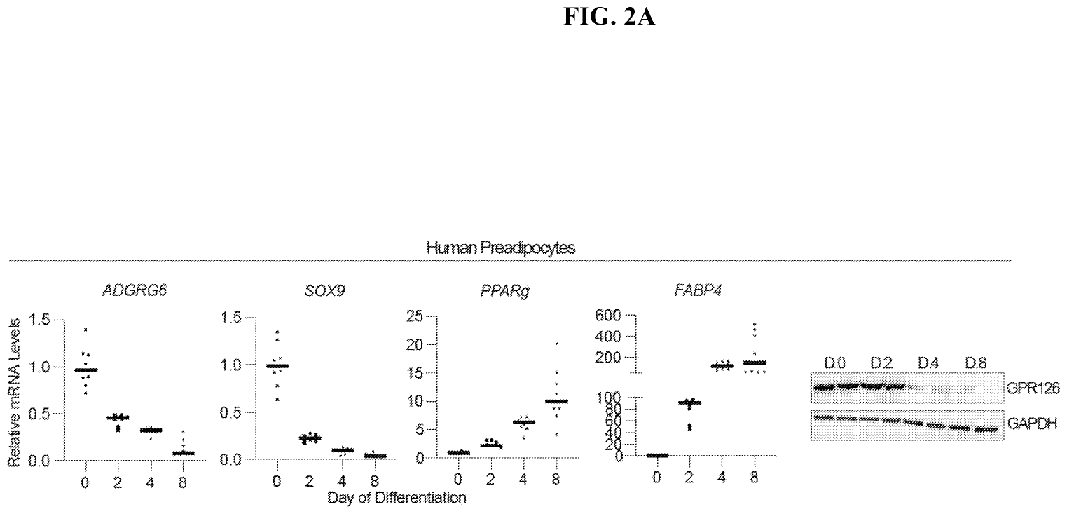

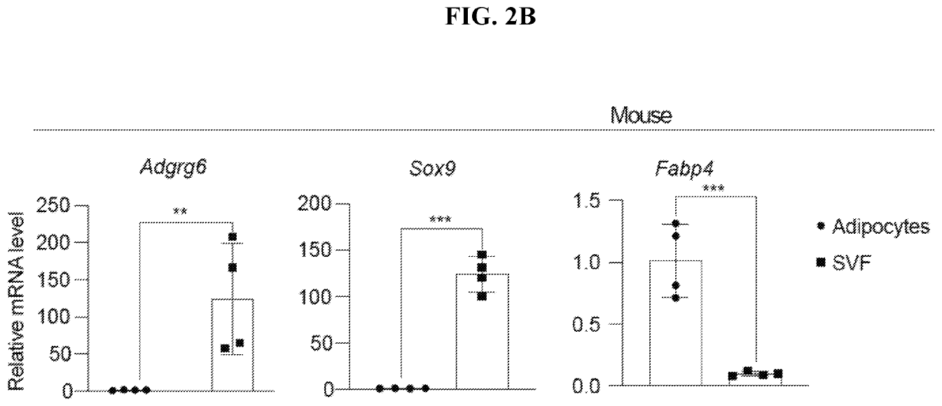

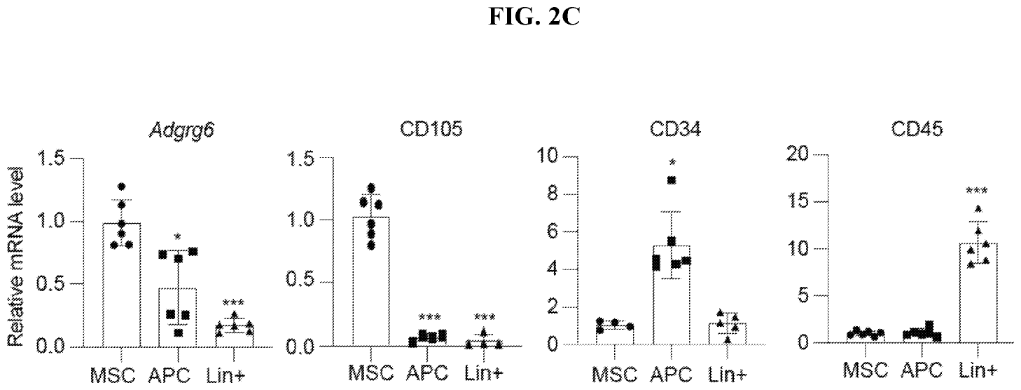

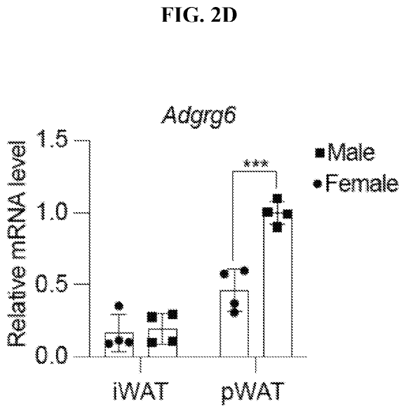

We next set out to examine the role of ADGRG6 in adipose tissue. We measured, via RT-qPCR, ADGRG6 mRNA levels during human adipocyte differentiation and found them to be the highest in preadipocytes and to significantly decrease when subjected to adipocyte differentiation (FIG. 2A). The expression pattern of ADGRG6 was similar to SOX9, which is required for adipogenesis and is known to decrease during adipocyte differentiation [58]. In contrast, PPARg and FABP4, known adipogenic markers which we used as controls, were significantly increased during adipogenesis (FIG. 2A). Analysis of ADGRG6 protein levels using Western showed similar results, whereby its protein expression was higher in preadipocytes and markedly decreased upon adipocyte differentiation (FIG. 2A).

We next examined Adgrg6 expression levels in mouse adipose tissues. We isolated the stromal vascular fraction (SVF) and adipocytes of pWAT of male mice after collagenase treatment. Gene analysis revealed that Adgrg6 mRNA levels were similar to Sox9, being 100-fold higher in the SVF fraction than adipocytes and in contrast to Fabp4 levels which were significantly higher in the adipocyte fraction (FIG. 2B). From the SVF, we further FACS-isolated mesenchymal stem cells (MSC) (CD105+), adipose progenitors (APC) (CD34+, Pdfrga+) and immune cells (CD45+). Within the SVF, we observed that Adgrg6 is highly expressed in MSC, indicating that Adgrg6 might be required for adipose lineage development (FIG. 2C). Overall, these results demonstrate that ADGRG6 is highly expressed in early adipose progenitors and MSCs and significantly decreases in expression during adipocyte differentiation, potentially playing an early role in adipogenesis. In addition, Adgrg6 shows a similar expression pattern in both humans and mice (FIG. 2D), suggesting that mice could be used as a model to characterize its function.

### Example 3. Adgrg6 is Involved in Adipogenesis

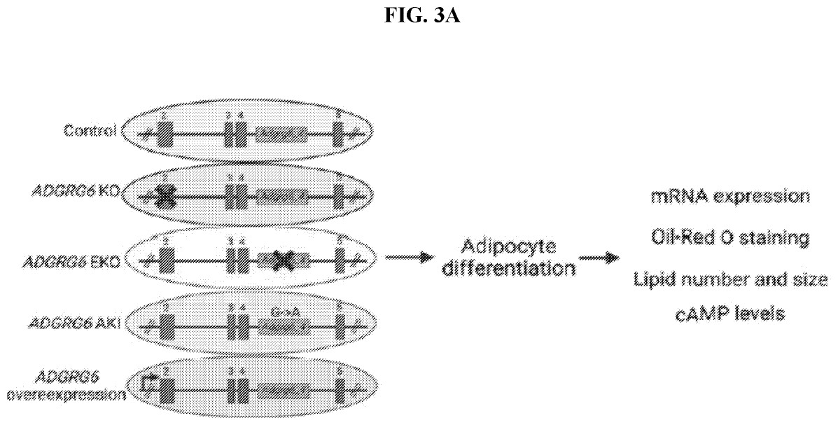

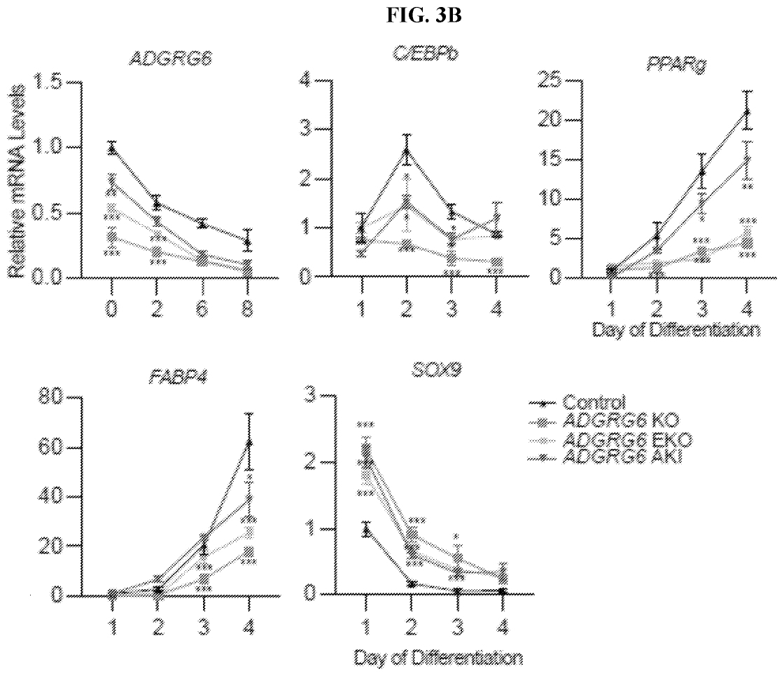

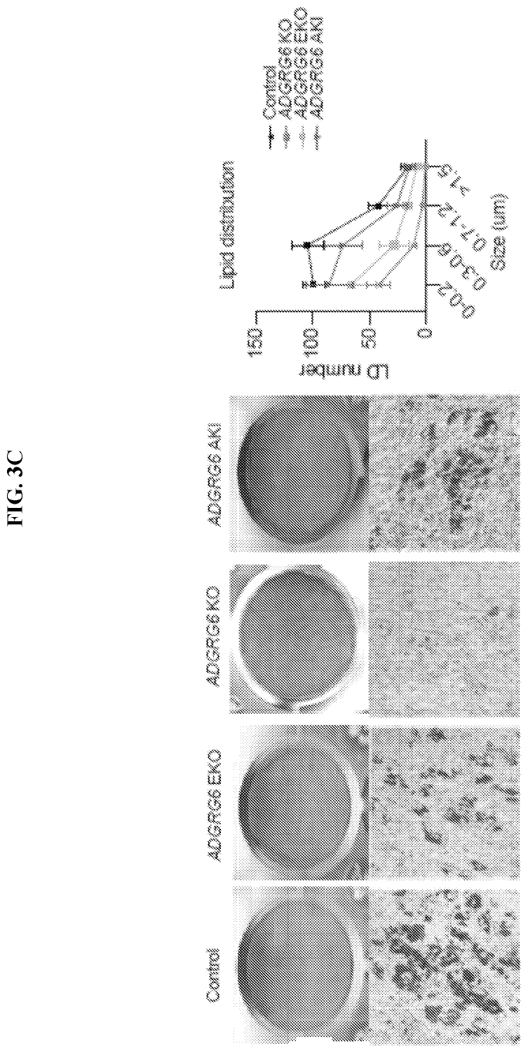

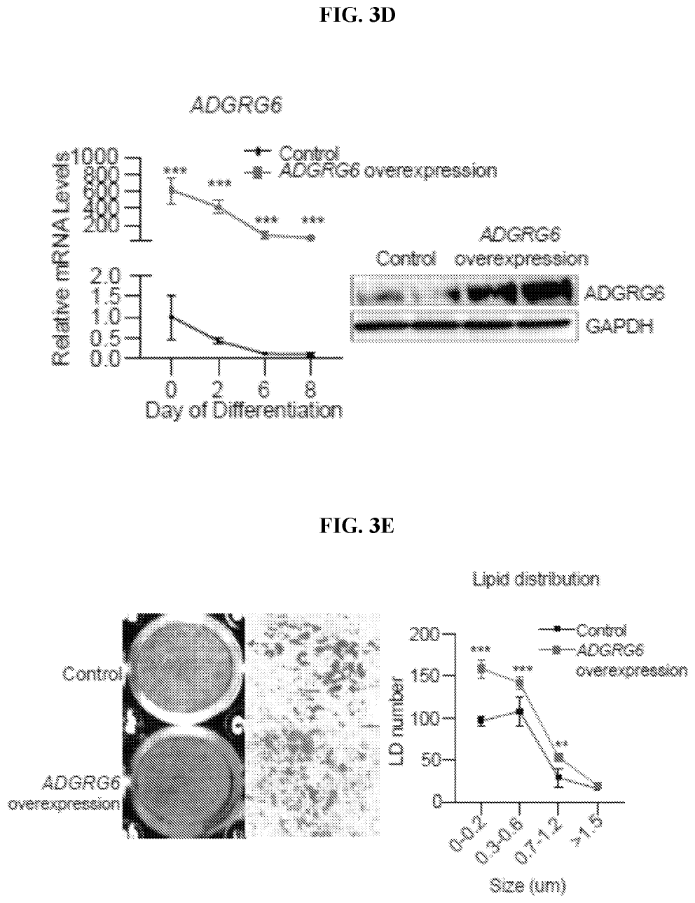

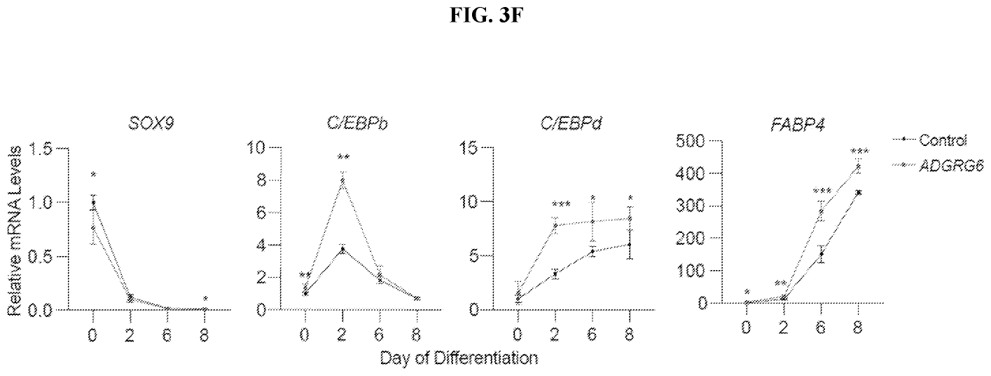

To characterize the role of ADGRG6 in adipogenesis, we knocked out the gene, the ADGRG6_4 enhancer, and generated a human preadipocyte line containing the associated rs9403383 SNP (FIG. 3A). For the gene knockout (KO), we transfected human preadipocytes with Cas9 protein and two gRNAs targeting exon 2 of ADGRG6. For the ADGRG6_4 enhancer KO (EKO), human preadipocytes were transfected with gRNAs targeting the two ends of the enhancer sequence, along with Cas9 protein. For the associated SNP knockin (AKI), we first sequenced the human preadipocyte cell line and observed that it contains the unassociated allele. These human preadipocytes were transfected with a gRNA targeting ADGRG6_4 and a ssDNA donor containing the associated SNP rs9403383. Single-cell clones for all three manipulations were FACS isolated and screened by genotyping for homozygous colonies. We then measured, using qRT-PCR, ADGRG6 mRNA levels finding the gene KO cells to have nearly depleted levels of ADGRG6 transcripts, EKO cells 50% reduction and the associated variant 20% reduction compared to wild-type (WT) cells (FIG. 3B). We then subjected the ADGRG6 KO, ADGRG6 EKO, associated variant and WT cells to adipocyte differentiation. mRNA expression analyses using qRT-PCR showed that both ADGRG6 KO, ADGRG6_4 EKO, and ADGRG6 AKI had lower expression of adipogenic markers, such as C/EBPb, PPARg, and FABP4 compared to the WT cells. In contrast, SOX9 levels, an adipogenesis inhibitor [58], were higher in ADGRG6 KO, EKO, AKI compared to WT cells (FIG. 3B). Oil red O lipid staining showed that KO, EKO, and AKI cells have lower lipid accumulation compared to WT (FIG. 3C). In addition, using Fiji [59], that quantifies lipid droplet size, we observed both ADGRG6 KO, ADGRG6_4 EKO, and ADGRG6 AKI cells to have more smaller lipid droplets containing cells than WT cells (FIG. 3C).

To analyze the effect of increased ADGRG6 expression on adipogenesis, we overexpressed ADGRG6 in human preadipocytes. Myc-tagged ADGRG6 was transfected into human preadipocytes, followed by their differentiation into adipocytes. ADGRG6 mRNA levels, as measured by qRT-PCR, were significantly higher in the overexpressing cells compared to WT cells throughout differentiation (FIG. 3D). Immunoblotting further confirmed the overexpression of ADGRG6 (FIG. 3D). In contrast to the knockout cells, ADGRG6 overexpressing cells exhibited higher levels of adipogenic genes (C/EBPb, C/EBPd and FABP4) and lower levels of SOX9 compared to WT cells (FIG. 3F). In addition, ADGRG6 overexpressing preadipocytes had much higher lipid accumulation than control cells and increased lipid droplet size (FIG. 3E). Combined, these data suggest that ADGRG6 has an important role in adipogenesis and deletion of its enhancer or the fat distribution associated SNP reduces its expression level leading to impaired adipogenesis.

### Example 4. ADGRG6 Increases cAMP Levels During Adipogenesis

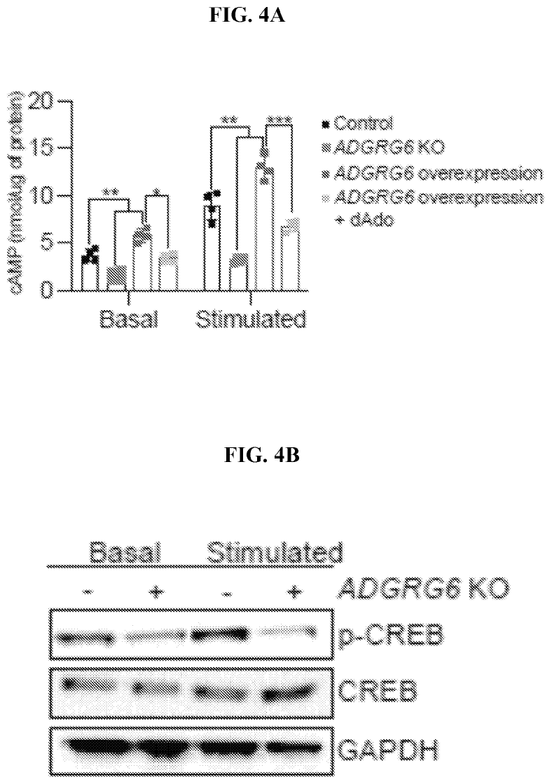

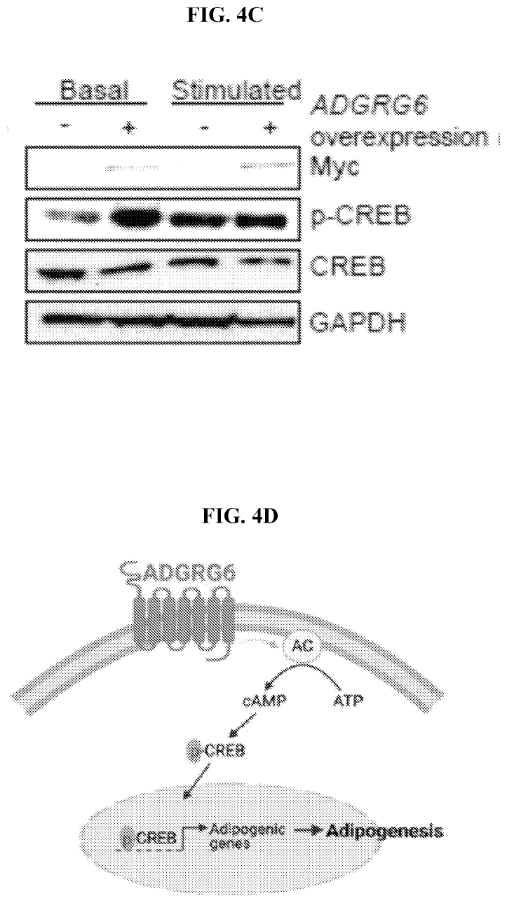

Previously characterized ADGRG6 ligands, such as Type IV collagen and Laminin-211, were shown to induce an adenosine 3′-5′-monophosphate (cAMP) response [42, 60]. CAMP is a well-known signaling molecule that initiates adipogenesis by increasing protein kinase A (pKA), which then phosphorylates early transcription factors, including C/EBPb [61-63]. To test whether ADGRG6 might increase cAMP levels in preadipocytes, we measured CAMP levels in WT and ADGRG6 KO human preadipocytes in both basal condition and forskolin which is an agonist pKA, using ELISA. We found that CAMP levels were approximately 2-fold lower in ADGRG6 KO cells compared to WT cells. In addition, upon forskolin treatment, WT cells had a 2-fold increase in cAMP levels compared to the basal condition while ADGRG6 KO cells did not show a significant increase (FIG. 4A). We also measured cAMP levels in human preadipocytes that overexpress ADGRG6. In the basal condition, ADGRG6 overexpression significantly increased cAMP levels and in the stimulated condition, cAMP levels were increased by 2-fold compared to WT (FIG. 4A). In order to test whether ADGRG6 can directly affect cAMP levels via adenyl cyclase (AC), that converts ATP to CAMP, we treated ADGRG6 overexpressing preadipocytes with 2′,5′-Dideoxyadenosine (aAdo), an inhibitor of AC. In the basal condition, cAMP levels were reduced in both WT and ADGRG6 overexpressing cells upon aAdo treatment (FIG. 4A). Moreover, in the Ado stimulated condition, the ADGRG6 overexpressing preadipocytes only increased cAMP levels by 25% compared to WT (FIG. 4A). Furthermore, we also examined the downstream target of CAMP-PKA, CREB phosphorylation. Immunoblotting revealed that in basal condition, ADGRG6 KO cells have lower levels of p-CREB compared to control cells (FIG. 4B), while ADGRG6 overexpression significantly increased p-CREB (FIG. 4C). In the stimulated condition, p-CREB increased in WT cells but not in ADGRG6 KO cells (FIG. 4B). In contrast, ADGRG6 overexpression did not further increase p-CREB (FIG. 4C), likely due to significant increase in the basal condition. Taken together, our result suggests that ADGRG6 increases CAMP levels and its downstream target CREB in human preadipocytes to promote adipocyte differentiation (FIG. 4D).

### Example 5. Conditional Knockout of Adgrg6 in Adipocytes Leads to Female-Like Fat Depots in Males

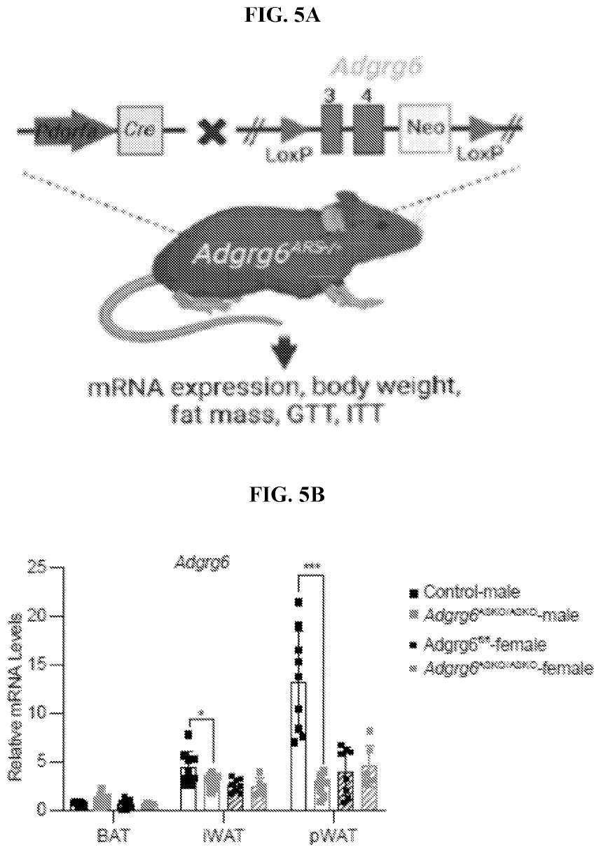

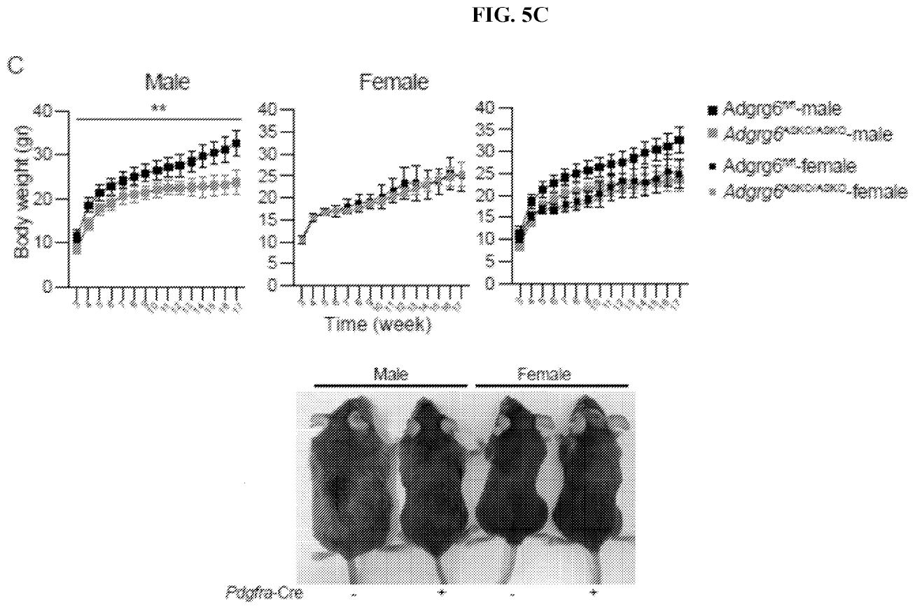

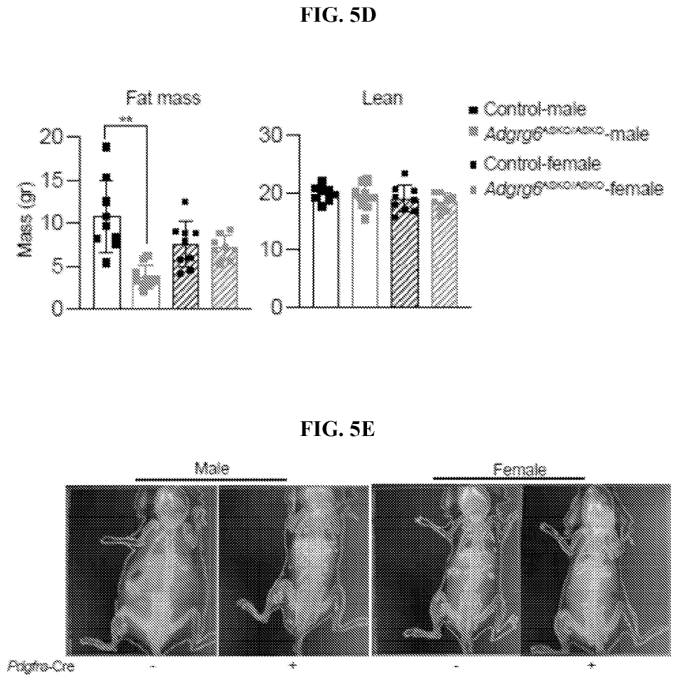

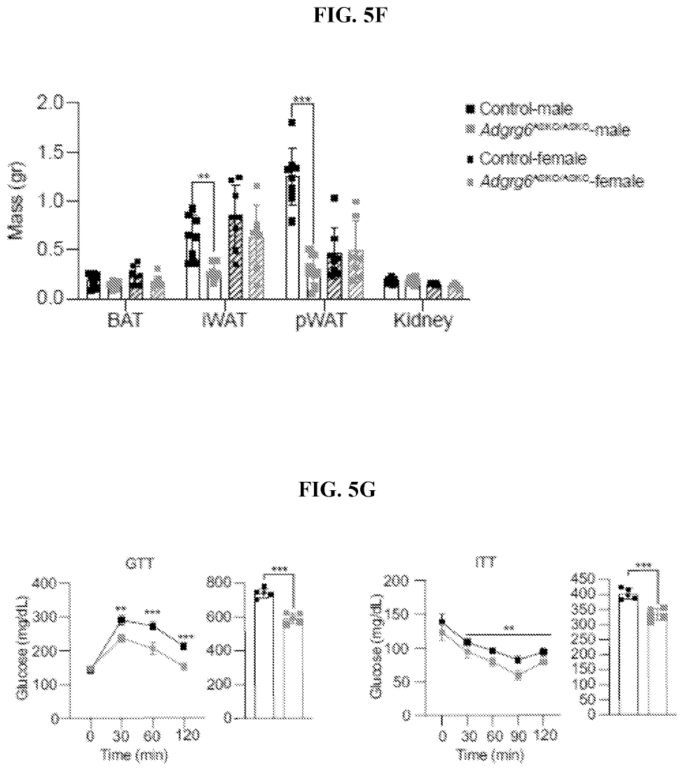

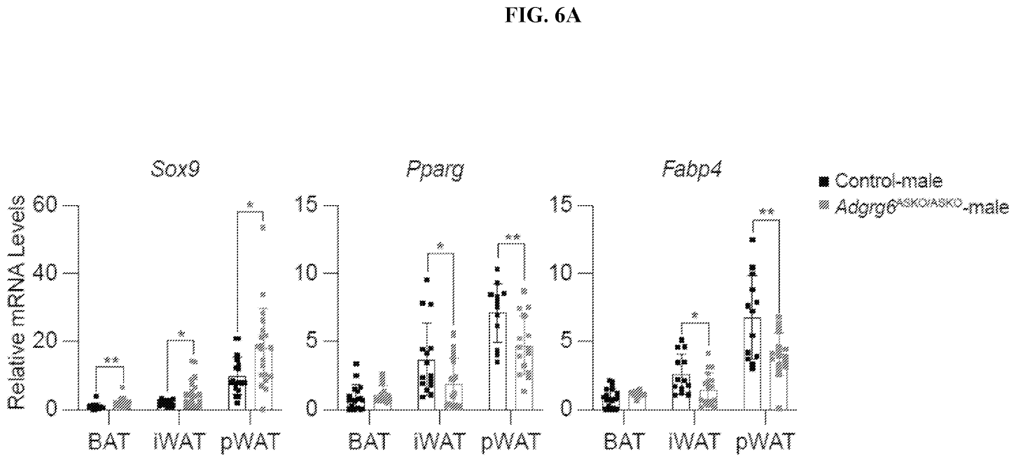

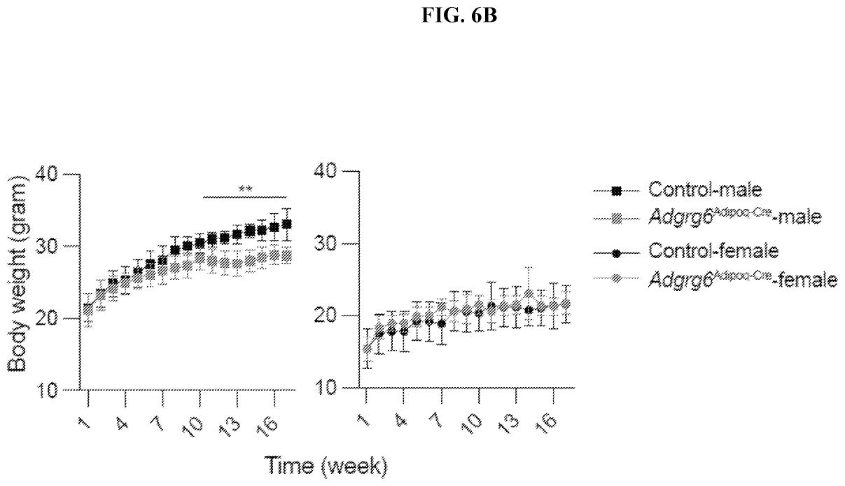

To examine the role of Adgrg6 on adipose tissue development in vivo, we generated adipose-specific knockout mice (Adgrg6^{ASKO}). We crossed loxP flanked Adgrg6 exon 3 and 4 (Gpr126^{fl/fl}) mice with the platelet derived growth factor receptor alpha (Pdgfra) promoter-driven Cre mice (Pdgfra-Cre), in which Cre is highly expressed in adipose-lineage cells and progenitors (FIG. 5A). qRT-PCR analysis of Adgrg6^{ASKO/ASKO} homozygous mice, showed Adgrg6 mRNA levels to be 80% in iWAT (inguinal) and 40% in pWAT (perigonadal) compared to the floxed Adgrg6 mice (termed hereafter as control mice). This ablation efficiency is reported in previous studies using the Pdgfra-Cre mice [66]. Notably, there was no significant difference in Adgrg6 expression between female Adgrg6^{ASKO/ASKO} and female control littermates (FIG. 5B). Adipogenic markers, such as Pparg and Fabp4 were decreased in iWAT and pWAT in Adgrg6^{ASKO/ASKO} male mice and Sox9, an adipogenesis inhibitor, showed higher mRNA levels compared to control littermate male mice (FIG. 6A). Adgrg6^{ASKO/ASKO} male mice showed significantly lower body weight (BW) than WT mice, while the female Adgrg6^{ASKO/ASKO} mice displayed no difference in BW compared to their control littermates (FIG. 5C). There was no difference in food intake between control and KO mice. However, the BW of the Adgrg6^{ASKO/ASKO} male mice was similar to age-matched female KO mice (FIG. 5C). The Pdgfra-driven promoter has been utilized to label and genetically modify adipose lineage genes, however, its expression is also detected in other tissues, such as the retina and glial cells [65-68]. We thus also conditionally knocked out Adgrg6 by crossing Gpr126^{fl/fl} mice with the adiponectin CIQ and collagen domain containing (Adipoq) promoter-driven Cre mice (Adipoq-Cre). The Adipoq promoter is known to drive expression in more mature adipocytes [69], however some studies utilized this mouse model to manipulate genes expressed in earlier adipose progenitors [70, 71]. Similar to the Pdgfra-Cre driven mice, homozygous Adgrg6^{Adipoq-Cre} male mice showed reduced body weight compared to control mice (FIG. 6B). Even though, the effect on BW was not as substantial as that of the Pdgfra-driven Cre, likely due to Adgrg6 expression being primarily in early adipose progenitors, the difference remains significant. All subsequent experiments were carried out in Pdgfra-driven Cre mice due to their stronger adipose progenitor expression.

We next used dual energy X-ray absorptiometry (DEXA) to examine body fat mass in these mice. We observed that the fat mass of Adgrg6^{ASKO/ASKO} male mice was approximately six grams lower than control littermates, with no difference in lean mass. There was no difference in fat mass between Adgrg6^{ASKO/ASKO} female mice and the perspective WT mice (FIG. 5D). Adgrg6^{ASKO/ASKO} male mice displayed similar fat mass to age-matched control female mice. Even though Pdgfra is not thought to be involved in cartilage development [72], as Adgrg6 has been reported to play a role in this process, we also measured bone mineral density (BMD) and observed no changes in skeletal abnormalities (FIG. 5E) and BMD in Adgrg6^{ASKO/ASKO} mice. Upon tissue dissection, Adgrg6^{ASKO/ASKO} male mice showed significantly lower iWAT and pWAT mass compared to control littermates, with no changes in brown adipose tissue (BAT) or kidney (FIG. 5F). No significant differences were observed between female Adgrg6^{ASKO/ASKO} and control littermates (FIG. 5F). As the BW of Adgrg6^{ASKO/ASKO} male mice was reduced and similar to control females, we next examined their glucose tolerance (GTT) and insulin sensitivity (ITT). We observed that Adgrg6^{ASKO/ASKO} male mice have significantly higher glucose tolerance than control mice at all measured time points, with significantly improved insulin sensitivity (FIG. 5G).

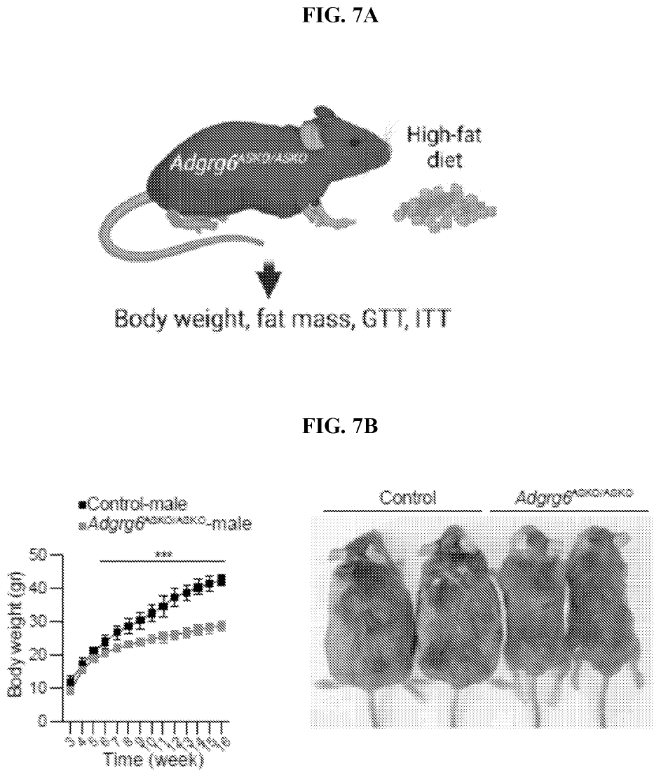

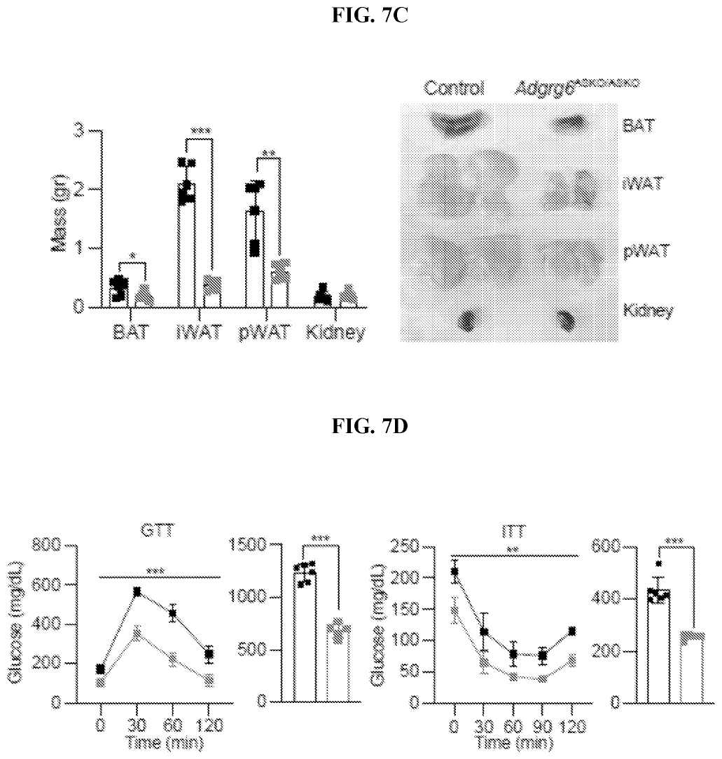

To further evaluate Adgrg6 ablation on adiposity, we placed Adgrg6^{ASKO/ASKO} male mice on a high-fat diet (HFD) (FIG. 7A). Compared to control mice, Adgrg6^{ASKO/ASKO} male mice gained significant less BW (FIG. 7B). In addition, the weight of all adipose depots, including BAT, iWAT, and pWAT of Adgrg6^{ASKO/ASKO} male mice was significantly lower than control mice with no changes in the kidney (FIG. 7C). In addition, Adgrg6^{ASKO/ASKO} male mice showed significantly improved GTT and ITT compared to control males (FIG. 7D), indicating that Adgrg6 ablation in males can reduce diet-induced obesity and improve obesity-associated insulin resistance. Taken together, our data suggest that Adgrg6 has an important role in adipogenesis in vivo and its removal in adipocytes leads to a female-like fat distribution in male mice, protecting them against HFD-induced obesity and providing improved insulin response.

### Example 6. Adgrg6_4 Enhancer Knockout Male Mice Have Female-Like Fat Depots

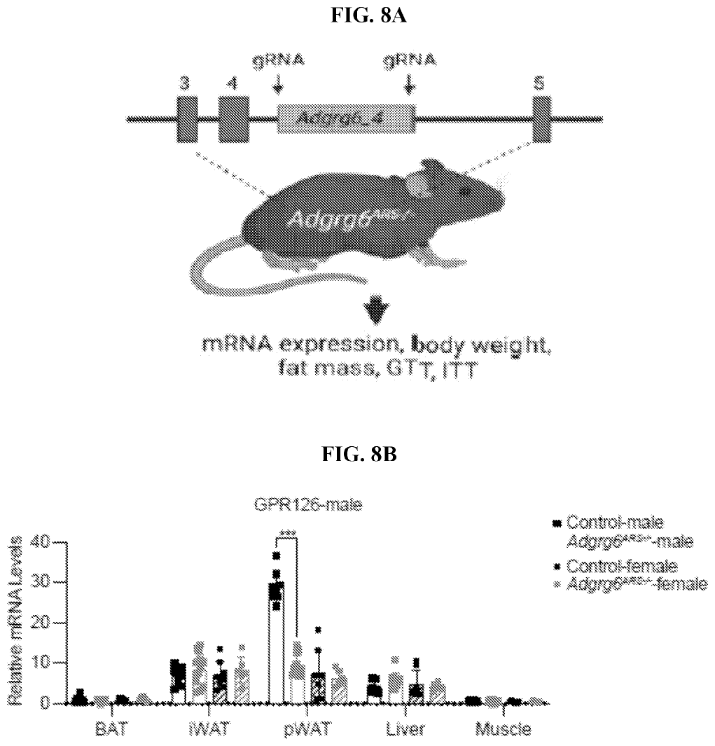

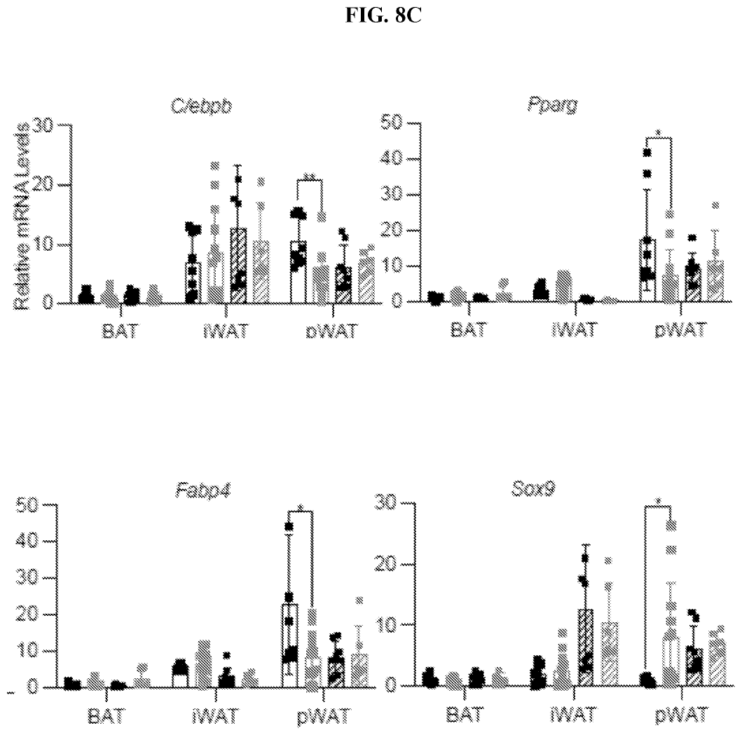

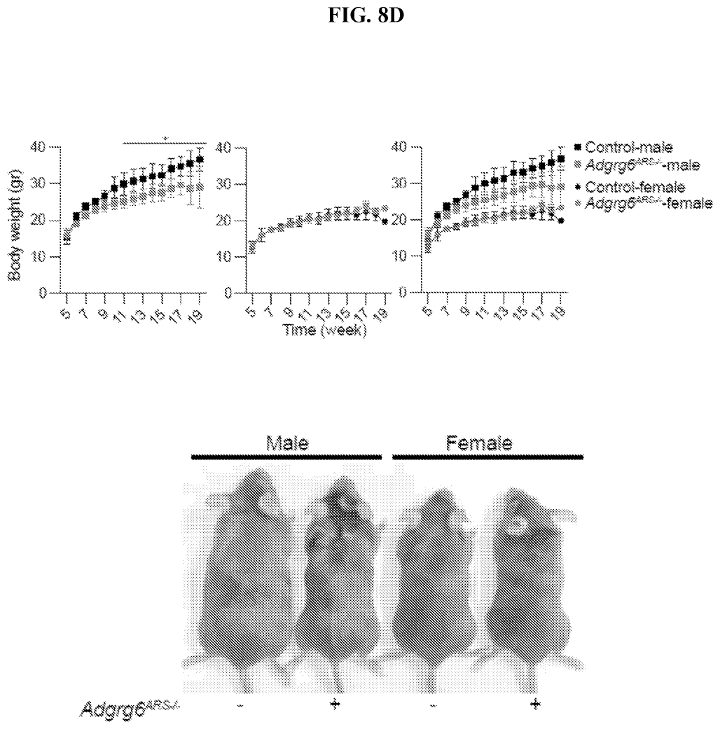

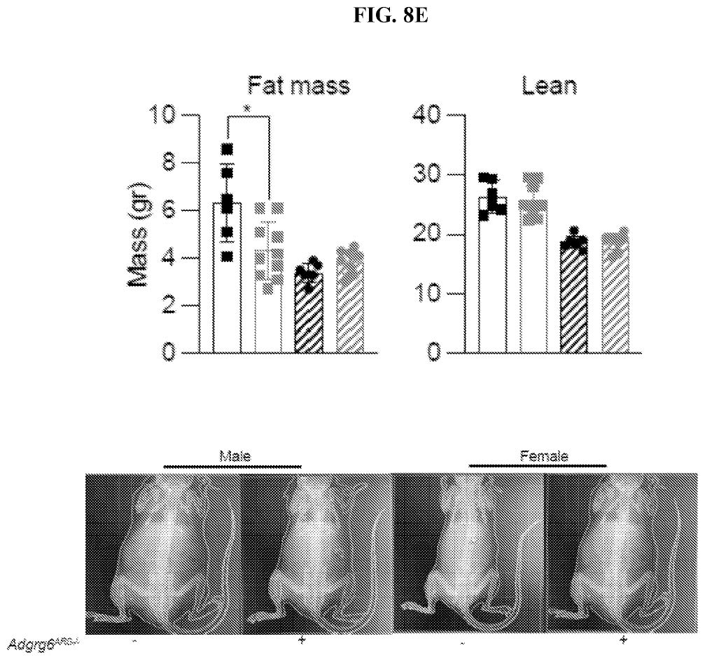

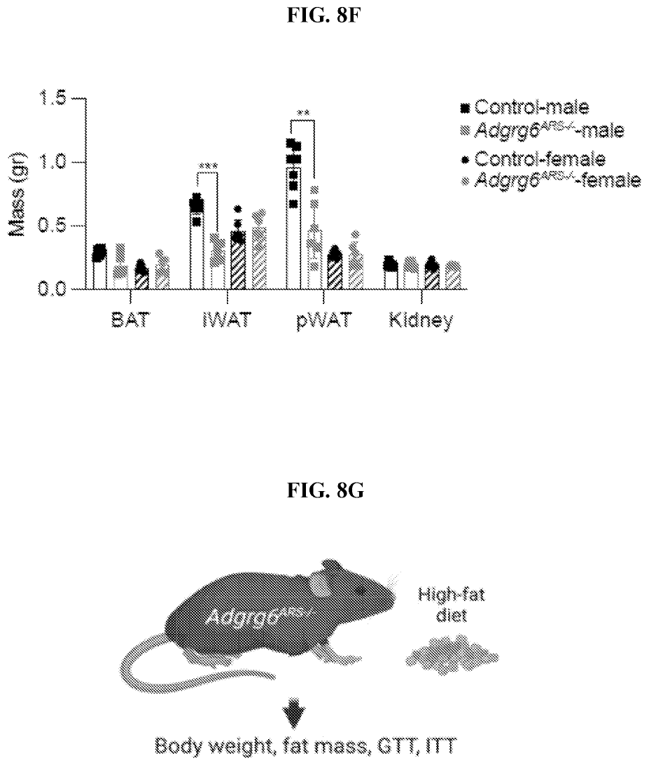

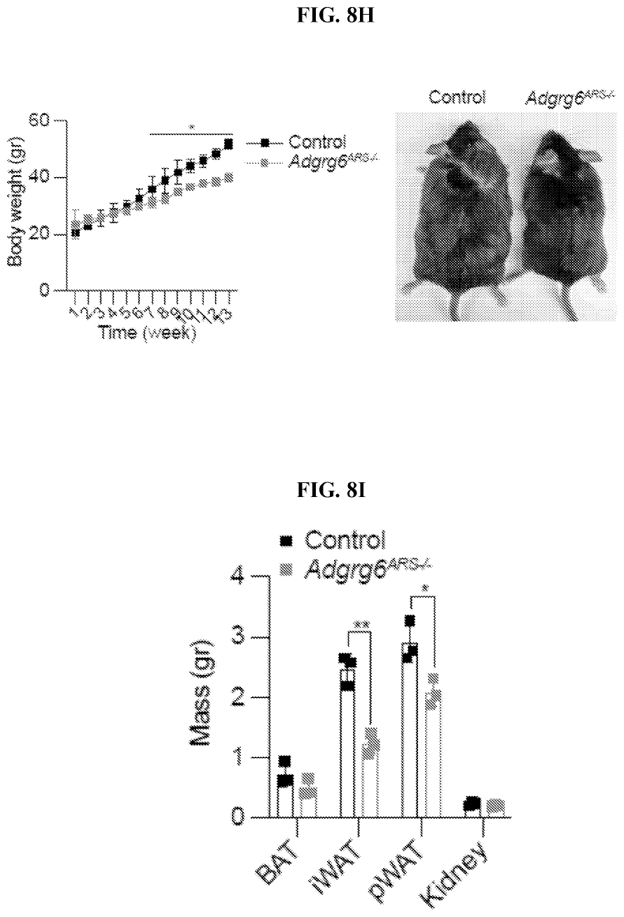

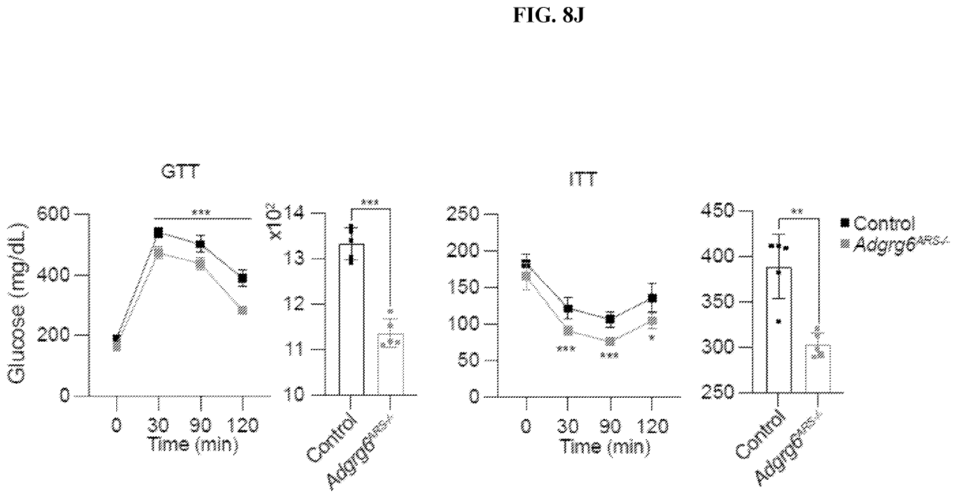

We next set out to characterize the in vivo function of the Adgrg6_4 enhancer by knocking it out in mice. We first identified sufficient mouse sequence homology to the human Adgrg6_4 enhancer using LiftOver [73]. We designed two gRNA to target the enhancer and generated Adgrg6_4 enhancer KO mice, which were named Adgrg6^{ARS-/-} (adipose regulatory sequence) using the improved-Genome editing via Oviductal Nucleic Acids Delivery (i-GONAD) approach [74, 75] (FIG. 8A). Knockout mice were validated for proper targeting via genotyping and Southern blot. Interestingly, analysis of Adgrg6 mRNA expression levels by qRT-qPCR, found it to be significantly reduced only in pWAT of male Adgrg6^{ARS-/-} mice (FIG. 8B). There were no significant changes in Adgrg6 expression in other adipose depots, including BAT and iWAT, and other tissues, such as liver and muscle. Moreover, Adgrg6 mRNA levels in female Adgrg6^{ARS-/-} mice did not change (FIG. 8B). These results suggest that the enhancer of Adgrg6 is only active in pWAT, indicating its tissue specificity. Analysis of the mRNA expression of adipogenic markers, C/ebpb, Pparg, and Fabp4 and the adipogenesis inhibitor, Sox9, showed changes similar to the conditional gene knockout only in pWAT of male Adgrg6^{ARS-/-} mice (FIG. 8C).

Similar to Adgrg6^{ASKO/ASKO} mice, male Adgrg6^{ARS-/-} mice exhibited lower body weight than littermate control mice, while there was no difference in female mice (FIG. 8D). Using DEXA, we showed that the fat mass of male Adgrg6^{ARS-/-} mice was approximately 2 grams lower than control male mice and similar to female mice (FIG. 8E). There was no difference in lean mass. Evaluation of bone mass and density due to Adggr6 known role in chondrocyte development found no changes in skeletal abnormalities in Adgrg6^{ARS-/-} mice. We next measured the mass of all adipose depots. We observed that male Adgrg6^{ARS-/-} mice had lower pWAT and iWAT (FIG. 8F). Even though, the changes only occur in pWAT, the effect in iWAT might be a result of whole-body lean phenotype. The Adgrg6^{ARS-/-} male mice did not show significant difference in GTT and ITT under chow diet. We next placed in Adgrg6^{ARS-/-} male mice on high-fat diet for 13 weeks (FIG. 8G). Adgrg6^{ARS-/-} mice had lower body weight than littermate control mice (FIG. 8H). In addition, these mice had smaller iWAT and pWAT (FIG. 81). Adgrg6^{ARS-/-} mice showed improved GTT and ITT compared to control males (FIG. 8J), indicating that Adgrg6 enhancer knockout can protect mice against diet-induced obesity and improve obesity-associated insulin resistance. Combined, our data demonstrate that the Adgrg6_4 enhancer is VAT-specific, has an important function in adipogenesis and leads to gender-specific effect on adipose tissue distribution.

### Example 7. CRISPRi-Mediated Adgrg6 Knockdown Prevents High Fat Diet-Induced Obesity

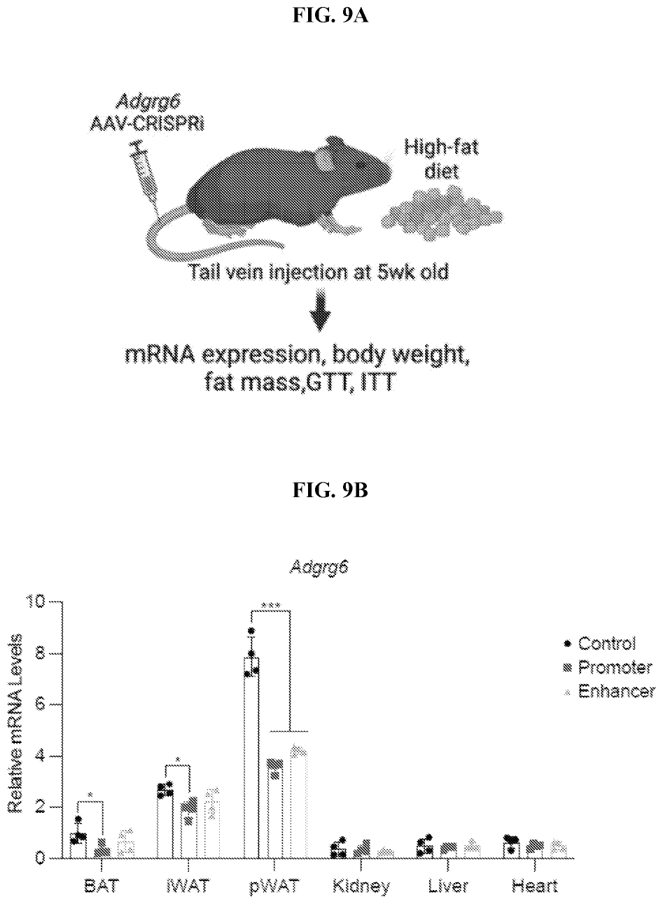

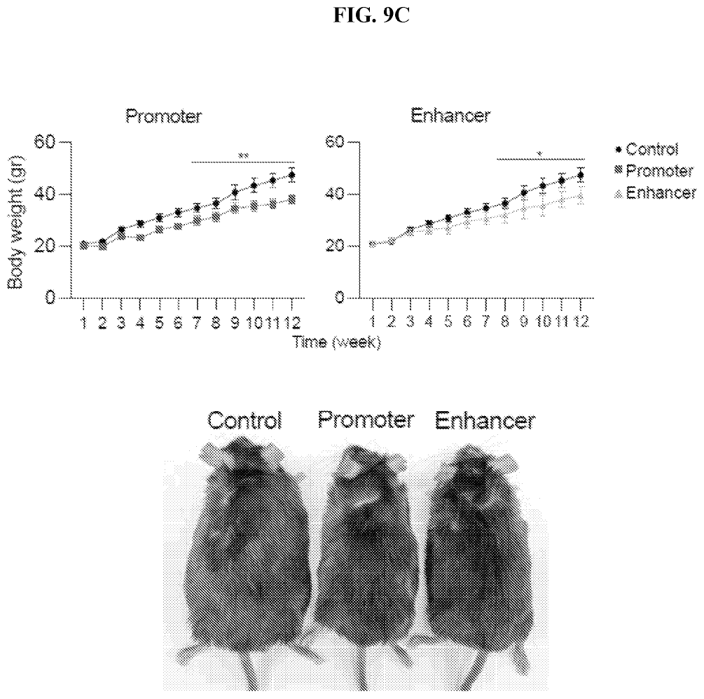

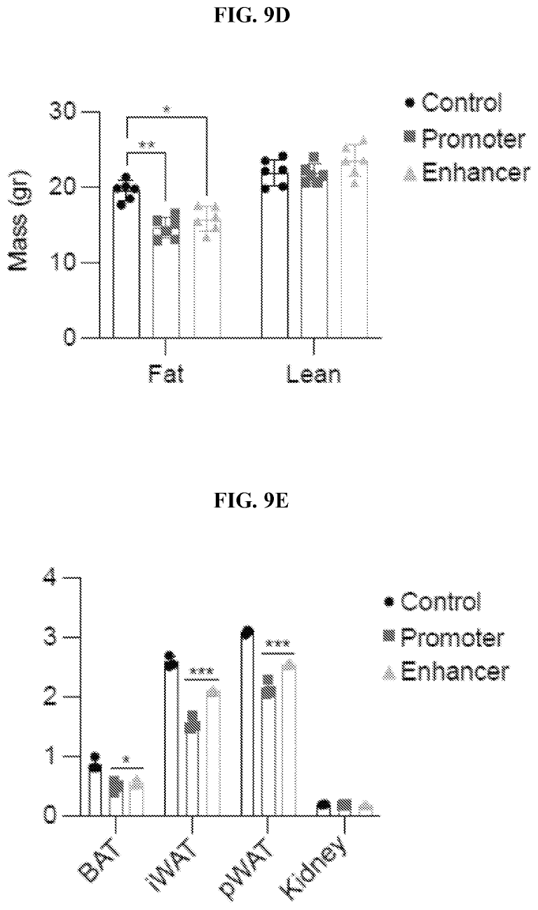

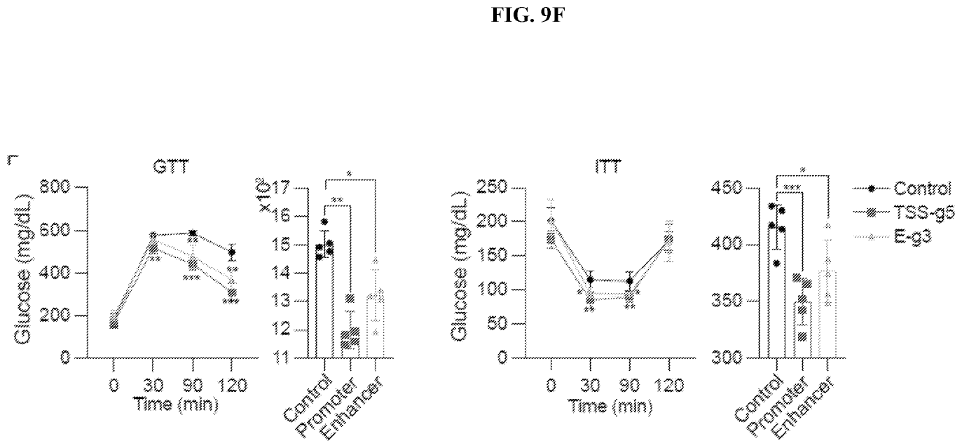

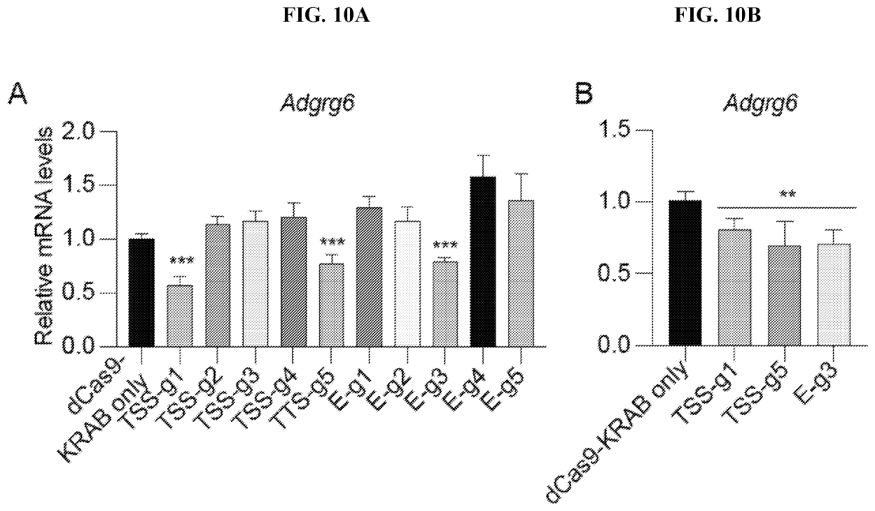

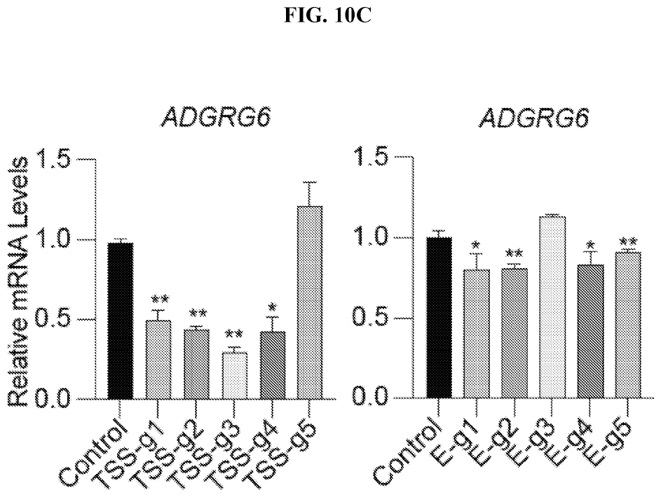

To showcase the therapeutic potential of Adgrg6 downregulation in males, we utilized CRISPRi, targeting either the Adgrg6 promoter or Adgrg6_4 enhancer, to knockdown Adgrg6 expression in vivo (FIG. 9A). We first designed five gRNAs targeting either the Adgrg6 promoter or Adgrg6_4 enhancer, cloned them into rAAV-mCherry vector and tested them along with a dCas-KRAB in mouse 3T3-L1 preadipocytes. We found two gRNAs targeting the promoter and one gRNA targeting the Adgrg6_4 enhancer to reduce Adgrg6 mRNA levels by ˜50-70%, as determined by qRT-PCR (FIG. 10A). The gRNAs along with dCas9-KRAB were packaged into AAV serotype 9, which is known to effectively express transgenes in mouse adipose depots [76]. Co-infection of individual gRNA along with dCas9-KRAB as AAV9 into 3T3-L1 cells found all three gRNAs to significantly reduce Adgrg6 expression (FIG. 10B). We also designed five gRNAs targeting either the human Adgrg6 promoter or Adgrg6_4 enhancer. We tested these gRNAs along with a dCas-KRAB in human preadipocytes. We found multiple gRNAs targeting Adgrg6 promoter or Adgrg6_4 enhancer were able to decrease ADGRG6 levels in human preadipocytes (FIG. 10C).

We next intravenously injected five weeks old C57BL/6J male mice via tail vein with a guide targeting the Adgrg6 promoter (g5 which showed the strongest AAV9-based reduction of Adgrg6 expression) or enhancer (g3) along with another virus encompassing dCas9-KRAB (control). These mice were fed on a HFD for 12 weeks (FIG. 9A). By qRT-PCR, mCherry and Adgrg6 mRNA levels were measured in adipose tissues, kidney, liver, and heart. mCherry levels were highly detected in all adipose tissues with the highest level in BAT and pWAT. This mCherry expression pattern is consistent with tissue distribution of AAV serotype 9, which has shown to effectively delivered to various adipose depots [76]. Notably, Adgrg6 levels were significantly reduced in pWAT in both CRISPRi targeting the promoter and enhancer of Adgrg6. The promoter mice also had lower expression Adgrg6 in BAT and iWAT than control mice (FIG. 9B). These data clearly indicate that our gRNAs can target expression of Adgrg6 in adipose-specific manner. This is due to the fact that Adgrg6 is highly expressed in adipose tissues. Thus, its promoter and enhancer are more active and accessible for gRNA and Cas9 complex. Previous studies using large-scale Cas9, and dCas9 cell culture screens have shown a targeting preference for regions with low nucleosome occupancy (active promoters or enhancers) [77, 78]. We observed a similar phenomenon upon CRISPRa targeting of Sim1 and Mc4r in mice [79], highlighting the tissue-specific therapeutic advantage of a cis-regulatory therapy (CRT) approach. Mice treated with CRISPRi targeting either Adgrg6 promoter or enhancer showed reduced body weight compared to ones treated with just dCas9-KRAB (FIG. 9C). Using DEXA, these mice also had lower fat mass compared to control mice (FIG. 9E). In addition, upon tissue dissection, we found that both the promoter and enhancer mice had much smaller BAT, iWAT, and pWAT than control mice (FIG. 9E). In addition, these mice showed significantly improved GTT and ITT compared to control males (FIG. 9E). All these data indicate that our CRISPRi targeting either the promoter or the enhancer of Adgrg6 can lower body weight and adiposity in mice and protects mice against high-fat diet obesity and diabetes, suggesting Adgrg6 might be a potent therapeutic target to treat obesity in humans.

### Example 8. Discussion

Body fat distribution is gender-specific and is a major risk factor for metabolic disease, including obesity, diabetes, and cardiovascular disease. Here, we found that ADGRG6, an adhesive G-protein coupled receptor, is more highly expressed in visceral fat of males than females. We identified an ADGRG6 enhancer that overlaps with a SNP that is associated with the visceral fat of females and show that the associated allele leads to reduced enhancer function and lower ADGRG6 mRNA and protein levels. We further show by either knocking out ADGRG6, its enhancer or overexpression that ADGRG6 promotes adipocyte differentiation by increasing cAMP levels. Adipocyte conditional removal of Adgrg6 or knocking out its adipocyte enhancer (Adgrg6_4) in mice, leads to decreased adiposity and obesity in male mice. These male Adgrg6^{ASKO/ASKO} or Adgrg6^{ARS-/-} mice are resistant to high-fat-diet-induced obesity with improved glucose tolerance and insulin sensitivity. CRISPRi targeting of the Adgrg6 promoter or Adgrg6_4 enhancer protected male mice against high-fat-diet-induced obesity, suggesting Adgrg6 is a potential therapeutic target to treat obesity and metabolic disease in males. To our knowledge, our study is the first to demonstrate a molecular mechanism in which a gender-specific SNP located in an enhancer decreases its activity and subsequent gene expression and results in gender-dimorphic effects.

There are several efforts to map the genetics of favorable adipose phenotypes and elucidate the causal mechanism of gender-specific genetic associations with central obesity and body fat distribution. GWAS provides a powerful tool to identify, in an unbiased manner, loci associated with complex traits and diseases, such as obesity. Large-scale GWAS and whole-exome sequencing efforts have mapped a treasure trove of novel findings for BMI, central obesity phenotypes, adipose distribution, and related metabolic traits. Interestingly, several studies have found that more than 50% of central obesity loci have significant gender dimorphism [37, 38, 80, 81], with the majority of SNPs residing in noncoding regions of the genome, likely associated with gene regulatory elements that reside in these regions. Here, we characterized one of these loci in the ADGRG6 locus, but there are many more to identify and characterize. It will also be interesting to assess combinations of these SNPs and how they might affect obesity and polygenic risk scores for this phenotype. Interestingly, rs6570507, is also associated with adolescent idiopathic scoliosis (AIS), which is more common in females compared to males [82-84]. It will be interesting to analyze whether loci influencing a gender specific effect in a certain phenotype could be used to inform regions that are more likely to have these effects also on another phenotype and how this mechanism evolved to influence multiple phenotypes.

Fat depot distribution differences is the major cause of obesity-related diseases [7-9]. VAT is highly correlated with obesity, insulin resistance and cardiovascular disease while SAT is shown to be metabolically protective against obesity-related metabolic diseases [4-6]. The differential location of fat has different functional implications on adipokine production, development of insulin sensitivity, inflammation response, mitochondrial function, lipolysis, and free fatty acid release, all of which differ between adipose depots [10-12, 14, 15, 85]. Our data showed that ADGRG6 promotes adipocyte differentiation by activating CAMP in preadipocytes. We also found ADGRG6 to have gender-differential expression in VAT in humans with VAT of males showing higher expression of ADGRG6 than females. Previous reports have found that numerous genes are differentially expressed in adipose tissue from obese males and females with very few on the sex chromosomes [35, 36, 86]. However, there is lack of understanding of how these genes are differentially regulated between genders. Here, we found rs6570507 to be highly associated with female trunk fat and reside in an adipocyte enhancer of ADGRG6. We demonstrate that this associated SNP alters the binding sites of multiple transcription factors, such as GR and PR that are involved adipogenesis [63, 87-91] and disrupts the binding affinity of these proteins, leading to lower expression of ADGRG6. Our work showcases how genetic variation can allow to identify an enhancer that can lead to gender-differential gene expression and potentially results in gender-specific fat distribution.

Ablation of Adgrg6 in adipose tissue using Pdgfra-or Adipoq-driven Cre (Adgrg6^{ASKO} and Adgrg6^{Adipoq-Cre}) or its enhancer (Adgrg6^{ARS-/-}) in mice all led to reduced BW and VAT mass in male mice but not in female mice. Male mice from all three deletions develop less VAT and exhibit similar metabolic phenotypes to female mice. Remarkably, ablation of the enhancer lead to the Adgrg6 mRNA levels reduced only in pWAT, indicating visceral fat specificity of the Adgrg6 enhancer. To date, Wilms Tumor 1 (WT1) is the only reported factor to be highly expressed in visceral fat and its promoter is used to drive Cre recombinase to conditionally ablate genes in visceral fat [93]. Now, our data clearly suggest that Adgrg6 enhancer can also be utilized to drive specific expression of a gene of interest in pWAT of male mice. They also have lower adiposity with improved glucose tolerance and insulin sensitivity than WT male littermates. Moreover, these male mice also became resistant to high-fat diet-induced obesity. Similar to humans, Adgrg6 expression was higher in VAT than SAT in male mice compared to female mice. Thus, the gender-specific body weight and adiposity phenotypes we observed in Adgrg6^{ASKO/ASKO} or Adgrg6^{Adipoq-Cre} might be due to the fact that Adgrg6 basal expression level is low in VAT of females compared to males. Deleting this gene or the Adgrg6 4 enhancer, that is conserved between mouse and humans, did not have a significant impact on VAT development in females. While further work needs to be done, for example in non-human primates, to suggest this gene and regulatory element have a similar effect in humans, our results provide strong support for its role in adipogenesis and gender specific fat distribution.

Cis regulation therapy (CRT), the use of nuclease deficient gene editing systems coupled to transcription modulating proteins has shown great promise to treat genetic disease [94]. For example, our lab has demonstrated that by upregulating via CRISPRa either Sim1 or Mc4r in heterozygous mice we can rescue their obese phenotype [79]. Gender-specific fat distribution contributes to differential metabolic outcomes and co-morbidities. Utilizing CRISPRi, we show that downregulation of Adgrg6 via CRISPRi by targeting either its promoter or enhancer could provide a viable therapeutic option for male obesity and its associated co-morbidities. Our data clearly showed that our gRNAs specifically target Adgrg6 in adipose tissues and can lower body weight, adiposity, and improved insulin sensitivity, Further development of CRT approaches that specifically target and downregulate ADGRG6 could provide a novel strategy to combat obesity and its co-morbidities in males. In addition, fat transplantation could also be considered for this treatment. As it is commonly used in many surgical procedures, such as aesthetic and reconstructive surgery, it could readily be used for therapeutic treatments [95]. In humans, there is a growing interest to carry out human adipose tissue grafting by using adipose stem cells/progenitors due to their resistance to trauma and long-term survival following transplantation [96-98]. Modulating ADRG6 in VAT and transplanting it in individuals might be novel way to treat obesity and associated metabolic diseases.

### Example 9. Materials and Methods

### UK Biobank Cohort and Sex-Specific Analyses at ADGRG6

The UK Biobank (UKB) is a large prospective study following the health of approximately 500,000 participants from 5 ethnic groups (European, East Asian, South Asian, African British, and mixed ancestries) resident in the UK aged between 40 and 69 years-old at the baseline recruitment visit [51, 99]. Demographic information and medical history were ascertained through touch-screen questionnaires. Participants also underwent a wide range of physical and cognitive assessments, including blood sampling. In the UKB, the trunk fat mass phenotype data (Data-Field 23128 on the web-site: biobank.ndph.ox.ac.uk/showcase/field.cgi?id=23128; description: Trunk fat mass in kg; category: Body composition by impedance; version March 2022) were collected on 492,768 participants. Phenotyping, genotyping, and imputation were carried out by members of the UK Biobank team. Imputation to the Haplotype Reference Consortium (HRC) has been described (www.ukbiobank.ac.uk), and imputation at a few non-HRC sites (for replication) was done pre-phasing with Eagle [100] and imputing with Minimac3[101] with the 1000 Genomes Project Phase I. We first ran a linear regression of trunk fat mass and SNP rs9403383 at ADGRG6 using PLINK [102] v1.9 (www.cog-genomics.org/plink/1.9/) adjusting for age, sex, and ancestry principal components (PCs). We then assessed the association between trunk fat mass and SNP rs9403383 by sex (245,000 women and 205,628 men, respectively, all of European ancestry). The analyses presented in this paper were carried out under UK Biobank Resource project #14105.

### Human Adipocytes

For adipocyte differentiation, human preadipocytes were cultured to 100% confluency in DMEM, supplemented with 10% FBS and fresh media were replaced. After 48 hr, cells were subjected to adipocyte differentiation by adding MesenCult Adipogenenic Differentiation Kit (Stemcell, 05412). Media was replaced every 2 days during differentiation. To generate ADGRG6 KO, ADGRG6 EKO, and ADGRG6 AKI cells, in a 6-well plate, human preadipocytes were transfected with 6,25 ug Cas9 protein (Fisher Scientific, A36498) and 800 ng sgRNAs (IDT), 1.5 ug ssDNA donor (IDT) (for ADGRG6 AKI) and 0.5 ug GFP plasmid (Addgene, 13031) using LipoMag transfection reagent (OZ Biosciences, LM80500) following the manufacturer's protocol. After 48 hours, GFP+ cells were isolated using (BD FACSAria Fusion), single clones were isolated into 96 well-plates. These colonies were then genotyped to collect properly edited clones. For ADGRG6 overexpression, in 12-well plate, sub-confluent human preadipocytes were transfected with 500 ng ADGRG6 expression plasmid Myc-DDK-tagged human G protein-coupled receptor 126 (Origene, RC212889) using LipoMag transfection reagent and cells were then subjected to the adipocyte differentiation protocol described above.

### Luciferase Assay

ADGRG6_3 and ADGRG_4 sequences were PCR amplified from the human genomic DNA, cloned into the pGL4.23 plasmid (Promega, E8411). Human preadipocytes, in a 12-well plate, were transfected with 0.5 ug luciferase constructs and 50 ng pGL4.73 [hRluc/SV40] plasmid (Promega, E6911) containing Renilla luciferase using LipoMag transfection reagent (OZ Biosciences) following the manufacturer's protocol. Empty pGL4.23 was used as negative control and pGL4.13 (Promega, E668A) with an SV40 early enhancer as positive control. Six technical replicates were performed for each condition. Forty-eight hours after transfection, cells were lysed, and luciferase activity was measured using Dual-Luciferase Reporter Assay System (Promega, E1910).

RNA Isolation and qRT-PCR

Total RNA from sorted cells or cultured cells was extracted using Trizol reagent (ThermoFisher, 15596026). Reverse transcription was performed with lug of total RNA using qScript cDNA Synthesis Kit (Quantabio, 95047) following the manufacturer's protocol. qRT-PCR was performed on QuantStudio 6 Real Time PCR system (ThermoFisher) using Sso Fast (Biorad, 1705205). Statistical analysis was performed using ddct method with GAPDH primers as control. Gene expression results were generated using mean values for over n=4-6 biological replicates.

### Separation of SVF and Adipocyte Fractions

SVF fractionation was carried out as previously described [103]. Subcutaneous and visceral mouse adipose tissue depots were dissected from 16 wk-old C57BL/6J mice into ice-cold PBS. The tissue was finely minced using scissors and digested with Collagenase type II in 3% BSA-HBSS buffer at 37° C. for 45 min with gentle shaking. The cell suspension was then centrifuged. The floating fraction containing adipocytes were collected and the pellet was resuspended and passed through 100 μm cell strainer and spun at 500 g for 5 min. The cell pellet was resuspended in HBSS buffer and passed through 70 μm and 40 μm cell strainers to ensure a single cell preparation. The cells were spun at 500 g for 5 min to pellet, and the red blood cells in the pellet were then lysed by incubating the pelleted cells in red blood cell lysis buffer on ice for 5 min with shaking. The remaining cells were washed twice with HBSS and spun at 500 g for 5 min to pellet.

SVF cells were incubated with indicated antibodies (e.g., antibodies targeting histone H3, glucocorticoid receptor, progesterone receptor, mouse IgG, Pparg, Gpr126, CD105, CD31, CD90, CD45, Ter 119, CD34, and CD140a (PDGFRa)) for 20 minutes in the dark, washed, spun at 300 g for 5 minutes, resuspended in PBS and passed through a 40 μm filter prior to FACS analysis. FACS was performed on ARIA Fusion Cell Sorter. Cells that were not incubated with antibody were used as a control to determine background fluorescence levels. Cells were initially chosen based on forward and side scatter (FFS, and CCS). Single cells were then separated into Lin+ cells (CD45+, CD31+, Ter119) and Lin-cells. In Lin-fraction, messenchymal stem cells (MSC) were then selected by CD109, adipose progenitors (APC) were sorted by CD34 and Pdfrga. Cells were collected in PBS for RNA isolation or in DMEM+20% FBS for cell culture.

### Oil Red O Staining

Differentiated adipocytes were fixed with 1% paraformaldehyde (PFA) for 20 minutes. Cells were then washed 3× with water and incubated in 60% isopropanol. Oil red O staining solution (3 mg/ml) was added to the cells and followed by staining for 15 minutes. Cells were then washed with water and images were obtained with a . . . . Lipid droplet size and number were analyzed by Fiji software [59]. There were 100 cells counted for each condition.

ChIP qPCR

ChIP was performed using ChIP-IT Express Chromatin Immunoprecipitation kits (Active Motif, 53009). Briefly, cells cultured on 15 cm plate were harvested and fixed with 1% PFA for 10 minutes. The reaction was stopped by incubating with 125 mM glycine for 10 minutes. Cells or tissues were rinsed with ice-cold phosphate-buffered saline (PBS) for three times, and lysed in IP lysis buffer. Nuclei were collected by centrifugation at 600×g for 5 min at 4° C. Nuclei were released by douncing on ice and collected by centrifugation. Nuclei were then lysed in nuclei lysis buffer and sonicated three times by 20 second bursts, each followed by 1 minute cooling on ice using Covaris m220 ultrasonicator. Chromatin samples were diluted 1:10 with the dilution buffer containing 16.7 mM Tris pH 8.1, 0.01% SDS 1.1% Triton X-100 1.2 mM EDTA, 1.67 mM NaCl, and proteinase inhibitor cocktail. Antibodies (2 ug) of Histone H3 (Abcam, ab4729), glucocorticoid receptor (Abcam, ab3671), progesterone receptor (Abcam, ab2765), and HoxA3 (Sigma-Aldrich, H3791) or normal mouse IgG (Diagenode, C15400001) and protein A/G magnetic beads (Thermo-Fisher, 88802). After the antibody-bead-chromatin mixtures were incubated at 4 C overnight, the beads were washed and cross-linked reversed. DNA fragments were purified, and samples were analyzed by qPCR for enrichment in target area of Adgrg6 enhancer using primers targeting 150 bp around the associated SNP. Four replicates were used for each antibody. The fold enrichment values were normalized to input.

Mouse lines

All animal studies were carried out in accordance with University of California, San Francisco ACUC and OLAC regulations. Mice were housed in a 12:12 light-dark cycle, and chow and water were provided ad libitum. Mice were fed with either chow diet or high fat diet with 40% fat (Research Diet, D12492i). GPR126^{fl/fl} mice were provided by Dr. Ryan Gray. Adgrg6^{ASKO/ASKO} constitutive adipose tissue knockout mouse was generated by cross-mating Pdgfra-driven Cre mouse (Jackson Laboratory, 012148) and GPR126^{fl/fl} mouse. Adgrg6^{Adipoq-Cre} mouse was created by crossing the Adipoq-driven Cre mouse (Jackson Laboratory, 010803) with the GPR126^{fl/fl} mouse. The Adgrg6^{ARS-/-} mouse was generated as described in [104]. Briefly, the 4 kb Adgrg6 adipose regulatory sequence was converted to mouse sequence using the UCSC Genome Browser LiftOver tool [73], Two gRNAs (IDT) (30 uM), designed to target the 5′ and 3′ ends of this region, were mixed Cas9 (1 mg/ml) protein and were injected into the oviduct lumen of female mice that were mated the night before. Subsequent offspring were genotyped via PCR-Sanger sequencing of the breakpoint and Southern blot.

### Sanger Sequencing and Southern Blot.

PCR-Sanger sequencing was preformed using standard techniques using three primers. For Southern blot analyses, genomic DNA were treated with AvrII (New England Biolabs, catalog no. R0193) and fractionated by agarose gel electrophoreses. Following capillary transfer onto nylon membranes, blots were hybridized with Digoxigenin (DIG)-labeled DNA probes (corresponding to chr2: 147,202,083-147,202,444; mm9) amplified by the PCR DIG Probe Synthesis Kit (Sigma-Aldrich, 11636090910). The hybridized probe was immunodetected with antidigoxigenin Fab fragments conjugated to alkaline phosphatase (Sigma-Aldrich, 11093274910) and visualized with a CDP star (Sigma-Aldrich, 11685627001) according to the manufacturer's protocol. Chemiluminescence was detected using the FluorChem E (ProteinSimple, 92-14860-00).

### Body Composition and Food Intake Analyses

Body composition was measured using dual energy x-ray absorptiometry (DEXA) by PIXImus Mouse Desnitometer (GE Medical Systems). For DEXA, mice were anesthetized with 2% isoflurane and measured for bone mineral density and tissue composition (fat mass and lean mass). Food intake was measured by using the Columbus Instruments Comprehensive Lab Animal Monitoring System (CLAMS) (Columbus Instruments). Mice were housed individually and acclimatized on powdered picodiet (PicoLab 5058) for 3 to 4 days, and food intake measurements were done over 4 to 5 days.

### Glucose Tolerance Test (GTT)

Mice were fasted overnight. Measure blood glucose of each mouse to get the value for O time point. Blood glucose level will be determined by Contour Next Blood Glucose meter (Contour, 7277) in tail vein blood. Tail snipping is used to get blood. Glucose (1 g/kg of BW) (Sigma-Aldrich, G8270) was then administered intraperitoneally. Blood glucose was measured at 30, 60, and 120 min after the injection.

### Insulin Tolerance Test (ITT)

Mice were fasted for 4 hours. Measure blood glucose of each mouse to get the value for O time point. Blood glucose level will be determined by Contour Next Blood Glucose meter (Contour, 7277) in tail vein blood. Recombinant human insulin (0.75 IU insulin/kg BW) (Sigma-Aldrich, 19278) was then administered intraperitoneally. Blood glucose was measured at 30, 60, and 120 min after the injection.

### CRISPRa AAV in Vitro Optimization

Five gRNAs targeting the promoter of mouse Adgrg6 or human ADGRG6 were designed using the Broad Institute CRISPick Tool [105]. These guides were individually cloned into pAAV-U6-sasgRNA-CMV-mCherry-WPREPA at the BstXI and Xhol restriction enzyme sites using the In-Fusion cloning methods as described in [Ref]. Mouse gRNA constructs and pCMV-sadCas9-KRAB were co-transfected in human preadipocytes while human gRNA plasmids and pCMV-sadCas9-KRAB were co-transfected into mouse 3T3-L1 preadipocytes, both cell lines were maintained in DMEM supplemented with 10% FBS. After 48 hr, cells were lysed with Trizol and RNA were collected and cDNA were made as mentioned. qRT-PCR were performed, and differential expression were determined using ddct method with GAPDH primers as control. The best performed two gRNAs capable to reduce the expression of either human ADGRG6 or mouse Adgrg6 were packaged into rAAV-9 serotype virons. These gRNAs AAV viruses (1×10³ MOI) and dCAS9-KRAB AAV (1×10³ MOI) were used to infect human or mouse preadipocytes. After 5 days, RNA was collected and cDNA were made as described. qRT-PCR were performed, and differential expression were determined using ddct method with GAPDH primers as control.

### AAV Production

rAAV-9 serotype virons were produced by transfecting AAVpro 293T cell (Takara, 6322723) with pCMV-sadCas9-KRAB (Addgene, 115790) or pAAV-U6-sasgRNA-CMV-mCherry-WPREpA ([79]) along with packaging vectors, including PAAV2/9n (Addgene, 112865) and pHelper vectors using TransIT293 reagent (Mirus, 2700). After 72 hr, AAV particles were collected and purified using AAVpro Cell & Sup. Purification Kit Maxi (Takara, 6676) and quantified by the AAVpro Titration Kit. (Takara, 6233).

### CRISPRi Mouse Injections

C57BL/6J mice at 4 wk old were kept under live anesthetic isoflurane at 1.0%-2.0%. AAV9-dCas9-Krab and AAV9-gRNA (1×10¹¹ vg/mouse) at 1:1 ratio was injected intravenously via tail-vein. Body weight was recorded weekly. Five-week post-injection, mice were subjected to DEXA, GTT, and ITT.

INFORMAL SEQUENCE LISTING

SEQ ID NO: 1: gRNA sequence targeting the Adgrg6 gene
ATCGAATGTGGAGCTGCCAT
SEQ ID NO: 2: gRNA sequence targeting the Adgrg6 gene
GGACAAAACCACTCGGCAGT
SEQ ID NO: 3: gRNA sequence targeting the promoter of the Adgrg6 gene
AGCTGAGGAAGTAGGGTGTGCGTGGG
SEQ ID NO: 4: gRNA sequence targeting the promoter of the Adgrg6 gene
GGCGGCAGGTCCCTCCTCGCAGGGAA
SEQ ID NO: 5: gRNA sequence targeting the promoter of the Adgrg6 gene
CCCTCCTCGCAGGGAAGTTGGCAGGG
SEQ ID NO: 6: gRNA sequence targeting the promoter of the Adgrg6 gene
CCTCGCAGGGAAGTTGGCAGGGTGAG
SEQ ID NO: 7: gRNA sequence targeting the promoter of the Adgrg6 gene
CCCTCCTCGCAGGGAAGTTGGCAGGG
SEQ ID NO: 8: Adgrg6 gene promoter
TGTACTTTCCACTATACACTCCATTTTCCTAACATGCTTTTTCAAAAGAAAAGAAC
AAATTCTTAGTTTGAGGACAAAACTGTCAAAATATACAGATTTTATTAACAAACT
GACCAATGAAAGGCACTGATTCATTGAAATATTTTAGCTATGATTCTGATCATAT
ATTAATTTCAATGCATAAAATTATGAACATTTTAACATATAATAATGGCACGTGT
GAAAACTACTATCTTCTTGATGTATGTCCATTTTTTTTCGGTTAATGTCATGGTTC
ATTTTATGTGTTAACTTGGCTTGGCCATGGTGTCCAGATTTGGCCAAACGTTATTC
TGTGTGTGTCTCTGATGGTGTTTCTTGGATGAGATTAACATTTGAATCAGTGGGTC
TTGCATAATGTCCTCTAGGTCCATCCACGTGTCACAAATGACAGAATTTCTAACT
TAAAGGTTGAACGGCATTCTGATGTGTATATATTCCACGTTTTCTTTACCCATTCA
TCCACTGATGGACAGAAAGAGAAATAGCAAATAATCTCACTTATATGTGGAATC
TGAAACAAACTCATAAGAGTAGAGAGTACAATGGTGGTTACCAGAGGCTAGAGG
GTGAGGTGGGGTGGAATGGTGAAATGTTGGTCAAAGGATACAAAGTTTCAGTTA
GAAGGAATAAGTTTTTGAAATCTATTTCACAGCATTGTGAATATAGATAATAATG
CCTTGCATATTTCAAAATTGCTAAATTTCAAATGTTCCTATCACAAAAATGATAG
ACATGTTAATTAGCTTGATATAATCATTCCACATGGAATAAAAATATAAAAATAT
CACATTGTATCCCATAAATATATACAATTATAATATGTCAATTAAAGATAAATGT
CAAAAAGAAAATCGGAATAAAAACTATTAAAAAATCAGTGGACAATGTACAAA
GAAGATTATTCTCCATTCTGCAGGTAGGCCTCATCCAATCAGTTGAAGGCCTTAA
CAGAACAAAGACTGATCTACCCTAAGCAAGAAGGAATTCTGCCAGACTGCATTT
GGAGTTGGACTATAACTTTTCCCTGAATCTCCAGGCTGCCTGACTGTAAATTTTG
CACTAGTACCTCCACAATACCTTAAAACTAAAATATATATATCTATGTGCATACA
CACATCTAGCTGGCTCCGTTTTTCTGGAGAACACTGAATAATGAAGTTATTTATA
TGTCTTTTCTTTTTTTGAGGCAGAGTCTAGCTCTTTTGCCCAGGCTGGAGTTCAGT
GGTGCAACCTCAGCTCACTGCAAGCTCCACCTCCCGGGTTCAAGTGATTCTCCTG
CCTCAGAAAAGGTAGCTGGGATTACAGGTGTGTACCACCAGGACTAATTTTTTTT
TTTTTTTTCAGTAGAGATGAGGTCTCACCATGTTGGCCAAACTGGTCTCGAACTC
CTGACCCCAACTGACTCGCCTCAGCTTCCCAAAGTGCTGGGATTACAGGCATGAG
CCACCGTGCCTGGCAGTTATTTATATTTATTTGCTCTTATGTTTCCAGGTTTCTTCT
AACTTTAAAATTCTATGACTGGAACAAATACATATGTGAACTTGGAAGTAGATCT
TTCCCCTGTCCAGCCTTCAGATCAGACCACAGCCTTAGTCACCACTCCAATTGTG
GCCTCCTGAGAGACACTGAGCTAAGGGCATCCAGGTTCTTGACCCACAAACTGT
GAAATACATGTTGCTTTTAAAAAATTCCAAGAGTGGCCCTTAAATTTCTTTGGGA
CTATACCGTGTAATAGCGACTGACTCAAAAGGGTAGTCTGGAAACCTAATCTTAG
CCCCTGGTCACCTCTAGCTAGTGTGCACAGACCTCAGATCTTGTCAGTCTCATTTC
CCTACCTGTTAACGTGAGACAGTTGGGCAGAAGAACTTTAGCTTCCCTTAAATGC
TACAACTCAGCGTCTCTATTAATCATACCGTTTATTCATCAAAATGAATACTGAT
AAAAATGACAAGTAGAACACACCCTAGATGTTCAGGCCTCTGGAATTGGTGGCA
GCGTTCCAGCTTCCTTTCTGCCTGTAACTGTGAGTAAGTCCTGCTTTACCTCCTTC
CCACCCCCCGCACCCACCAACAGCTCTCTATCATAAGCTAATCAGCTTGAAATAA
TTGTTAGGGCACTTCTGTGTATGGCCTGTGCATGTGCCAGTGTGTACACCGTGCA
AGCTGAGCTGTTGAGGTTCTGGATGTGCATACAGCCCTGGCACCGGAAGGTTTGC
CTCTAGCATTTTGTGGACTAACTCTGGCAGTACACAATGGGAAGAAAACCTCTTT
CCAAGTGTGAGCCCTTCTCTCTTCTCATTCCCAAGGAGGTGTGTCCAGATCCCTTT
GCCAGACAGCTACTTTACAGGAGCATTGGAGAGCAGGCAGCTAACTTTCCCCAA
CAAGCAGCTGGATTTACGTTGGCAAATCAATCCCTCTCAATAAAATGTTATGTGA
CCATCTTCCCAGCCCCCCGTTAAAATCCCTTCATTTTTTCGAGCAAGCGAACTAGT
CAGAGAAGACAGAAAAGTTAAGTAAAGAAAGTGGCAGAAAAGTGAAGCAAAGC
AACAGCCCGGGAGCGTGGCGGGGGGACCCGCGCGGGGACTCGGGTAGGCTGCA
AACGCTGGGGAGATGCCACAGGAGCGCCTGCCTTCCCAGCTTCTCCTTTCTCTCG
CCGAGGGTCTCTGGGGTTCAGCGCTCCCGGCGTTTCGGCGACCTCCGGGTCCGCC
CTGTCCCCTCCCAGCTGGGGAGCTGAGCCCGGCTCTGCGCCCATCGCCGACCCCG
GCCACTCTCCCTTCGCTGCTCGCTCCAAAAGCGCCTGTCCCGCCCCGCGGGGCGA
CTTCTCCCTTTCGGCCCTGGGGTTGAAGATCCTGTGTGGGGCAGCTGCGCCTTCG
GGACAGCCCCAGCCGCGGAGCGCCCAGACCAGGGCAGCCGGAGTCGGGTCATAC
GCCGCACGCCGCCGGGGCCGCCGCACCTGAGCCCAGCTCCAGTCACGCCGGAAG
CGCGGGCGGAGCGCACGGGTCCGGGGAGAGTCGCCTGCGCCCGCGAGGGGGAC
CCAAGTACCTAGGGTGGTGGCCGAGTCCCGCCTCCCGCCAGCGGGGGCGAGGAC
CTGCGACGCGCACCCCTGCCTGGCCCGGTCTCCTCAGCACCAGCCCCACGCACAC
CCTACTTCCTCAGCTTCTCGCCCTCACCCTGCCAACTTCCCTGCGAGGAGGGACC
TGCCGCCAGCCTGCTTCCTCGTCCGCAGGCCCTGCGCTGAACGCTGCCGCGCCCA
GGGTTCACCTTGCGCCGTCGGGAAAGCCCATGAACTCTCCAGAAACGGCGTAAA
GGAGGGTCCCGCCGCGGCGCAGGGCTGGGGCGCCTGGGTTCCCCCTGGGTGGAG
CAGCGGCAGCAGAGCGGGAAAGTGGTGGAGGATGATCTTGCGGCCAAAGGGGA
CCTCGGCGCAGTAATGTCAACATGTAAGTCTCACCTTTCAGCTTGGAATTCCTTC
ACTCTTTTATCTGTGTCCACCTTCTGTCGCCCCCTCCCCGACCACCCCACCCTCAA
GAGAAATGATTTTATAAGGACTTCAACTGCACGGAGGGAATAAACCGGTTTGTCT
TCAGTTTGCCCCTCGAGGCACTGAGGAGTAGGACTATTGCCAAGTGGAGTTGTGA
CATGTGTGTGTGGCTGTTTTTTTTCCCCCAGCGAGGTAAACCTCACATTTGGGCG
GTGTAAAGCCTAATAAGAAGGGCAGGGCTTCAAGAATGGGGAAAACGTGTTTTC
AATTCAAATTAAACAAGGAGTAACAGGCACCGCTCCTCAGTCCAGAGGCCTGGC
CCTGCCAAGAAGGCGCTCTCCGGAATCAACACCTGGGGGCTTGGAAG
SEQ ID NO: 9: gRNA sequence targeting the enhancer of the Adgrg6 gene
CCCGTAACATGGGATCTATGGTGTAG
SEQ ID NO: 10: gRNA sequence targeting the enhancer of the Adgrg6 gene
CCCAGAATGCAGGAAGTGCACCATTC
SEQ ID NO: 11: gRNA sequence targeting the enhancer of the Adgrg6 gene
CCCCTGAATTAAAGACAGTCACCCAG
SEQ ID NO: 12: gRNA sequence targeting the enhancer of the Adgrg6 gene
GTCTATGGTGAAAAGAGACCCCTGAA
SEQ ID NO: 13: gRNA sequence targeting the enhancer of the Adgrg6 gene
CTCTAATACCAAACTTTCCAAGCTCC
SEQ ID NO: 14: Adgrg6 gene enhancer
TCTCCATTTTTTTTAAAATGAGGATTTATAGTTTGTTTCAAATACTGTGCTGTCTCT
GGTTCTTCTTTGTGAGACTGTAAATGTGCTATTATTGGAAGTAGCTAAATGCAGA
ATATTTTGAGAAATAATTCTGTGTGATAAAAATTGTATTAAAATAGATCTCCACC
TAATATTCTCCATTTACACAAGTTAATTCTGGAAGCCTTTATTTTATTATAGGACT
GATTCAAACACAAAAAGTAGAAGTTTCATTATCAACTTTAAATGTCATATGACTA
AATCTAAGGCAGTGATAATATATACATTTTAGTCACAGACATAACTCTCAATTAA
AAAGGAGAAGAAGGAGCCTAGTATTCAAAAGTAATTTGTATATGATATTGGAGG
GAAGTTTACTGGGAATTTATTCCCACGCTTTGAGCAAAGTAGCACCCTTATGATG
ACTCAAGGCCACCTCCCAATTTCTCAGACCCATGCCTGCCCTTCTGGCCTGAGAA
CTCCTGTTACCCACCTGTTCGTGTGGTCCATGGCACTCCTACAGGTGGTGGAAAG
GCTGTGAAGTGATGTCATTTGAAAGATATTTCCTTGCAGGATGTTGGAAAGTAGA
TAAACCACAACCCTGTTCCACAAGGGAACTGCAGAGCTTTATAAGAGCTGGTGA
TGTTCCCTAAGATCTTCTCTCAGTGTTAGGATAGCTAGAAAGGACCTTCAGGGAC
TCCTTGAGAAGTCTTTTATCAGAGAAGAAATTGAGAAAGTCAGGTGTGTATGTTT
GGTGTGGGACAGTGAAGAATAAGGAGAGGGAATGGTGCACTTCCTGCATTCTGG
GTGACTGTCTTTAATTCAGGGGTCTCTTTTCACCATAGACTGGGACATTATTAAG
AAGTCATAGTATAGACCATTCTAATATTTGCCTTTTTATGGGACAATGTTTTGAAG
ATGTCAGCAACCAAAACAAATATGGCTATGTTAATAATTGCTAATACTTATATTT
AAATTGAACACACATTTTGTACTAAATGTCATTTTAAGCACTTAACACATATTGG
TTGATTACCTACACCATAGATCCCATGTTACGGGTTCTATAATTGATCCTATTTTG
CAAATAAGGAACCTGAGGCACGTGGAGATTAACTAACTTATCCAGTGTCTCTTAA
TTAGTAAGTAGCCAAGCCAGGATTGGAGCTTGGAAAGTTTGGTATTAGAGCTGTG
CTCTTCTAACCAGTATAAATTAGACTGTCATTCATCATAAACAGGATTAGATATT
AGATTTAGTACCAGGATAGGCTAAAGCTCATTCTTGCTCAGGGGAGAAGTATGA
AGGTCCAAGAGCCATCTCTCTGGCCAGCAGTGCATTCTGTGAGAAAACACTAGG
TTTGCAGTGTGTTAACATATTTGTTTCAAACAAATAATATTTGTTTCCCACAATAC
TTCCTGGATACCTAACTATGAAAAAGTATGATTCAGTTTTACAGGGATTCCTAAG
CTTCTCTAAGAATTTCTCTAGACCAGAGGTTGGCAAATTAGGAGGGCCTGTAACA
GGCCATGCATTTTTGTTTACTTGTTTGTTTAGAGACGGGGTTTCACTCTGTCCCCC
AGGGTGGACTGTAGTGGTGCCATCAGAGATCGCTATAACCTTGAACTTCTGGGCT
CAAGTGATCCTCCTACTTCAGCTCTCCTGAGTAGGTAGGACATCAGGTGTGCACC
ACCATGCCCAGCTAATTTTTTAAAACATTTATTGTAGAGATGAGGTCTCACTGTG
TTGTCAAGGCTGGTCTTGAACTCCTGGCCCCATGTGATCCTCCCTCCTCAGCCTCC
CAAAGTGGTGGGATTACAGGCATGAGGGATTGTGCCCAGCACACATGTTTTTTTA
ATACTTGTTTTTCTTTTTCTTTTCTTTTTTTTTTTTTTTTTTTTTTTTAAGTTGGGGTC
TTACTGTGTTGCCCAGACTGGCCTCAAACTCCTGGGCTAAAGCAATCTTCTAATC
TCAGCCTTCTGAGTAGCTGGGACTAAGCTCCCTTGTTCTTATAAATAAAGTTTAAT
TGGAACACAGCCATGCCCATTAGTTTATATATTGTCTTTGTCTGCCTCTGTGCTAC
AATGGCTGTGTTAATTGATTGTGATCAATACAGTATAGCCTGCCATCCTAAAATG
TTTACAGTCTAGCCCTTTAGGAGAACATTTGCCACCCCCTGAAACTAGTCTAAAG
AACTGAGAGTCCGCTTTGCTGAACCACATACTTTGGACAGCACTAGAAAAGACC
TGCTAGATCTCCTCAGCCAGGGAAAACAAACCAATCACCCACAGGCTAGTTTAG
CCCTACGACATGGGTGGTGGTAGTAGCATTTATTTGTTCCTGTTCATTTACATTTG
TTTCACACAGTGCTTTTAATTGATGAATTATTTCCAATATTAAACAATTAGATTTC
ACATAAAAATGAAGTTTTGGATTTTTCTGGTAAACAGAATACACTAGGCTACTAT
ATCTGCATGGCCAACACAGCTGGAGCTAAATGGTGGCTGCTGCTTTCAAAGGAG
CTTCCCAGTTTACTTTAGCCCTTAGCAACCCCTTATTCTTCCCACTCCAGCCCCCT
TTTTGTCTTTAGATGGCCATCTGGCCCTGATGGGCTTTTAAATGGGAATGCTGATT
TATGCCACTCCCTTTATACGGGCCAGGAAACTGAAGCTTAGACAAAAAAAAGTG
GCAGAGGCAGTTGGTGGGCTCCAACACGTGTCTCCTGTCTCCTTGTCTAGTATTT
ATGTCTGGGAATACTCATTTAAAAAATTATTTTAAATTATTTTAATTGATTGATAA
GAACTATATGTGTTTATGGTATACAACATGTTTTAATATAGGTATACATTGTGGA
ATGGCTAATATGAGCTAATTAACTTTCATTGTTATCTCATATACTTATCATTTTAT
TTGTAGACAGAACATTTAAAATCTCTCAGCAATTTTCAAGTATACAATCATTGTT
ATTAACAATAATCATCATGTTGTACAATAGATTTCTTGAATGTATTTCTCCAGTCT
AACTGAAATTTTCCATCCTTTGACCCATACATTCTCAACTCCTGTGCCCCATCCAC
CCCTGTGCCCCTGGAAACCACTCTTCTATTCTTTGCTTTTATGAGTTCAACCTTTTT
AGGTTCCACATTTAAGTAAGATCATGCAGTTCTCATAATTTCTTAAACTCTGTCCC
TGAGACTCCTTTCCCAAGCCTTAAGATTATCGGAGTGCATCAAATACATACTCAT
GTATCTTAGTCATCTATAAGTTTTCAAAGTAATTTTGTCAAAAGGGTACCGTGAA
CTTAACGGAGTCAATATATTTGAGTCTTTCTAACTTTTATAGTGATCTTCTTGCCA
TTCCAAGTTGACGTTAAACACTCACATTCCAGACATTCAGATGTCCTGGACTGTG
TTTCAGGAGAAATGATGGGAGAGGGAATTTCCTGCTGCTTAATCATCAGCTTTGG
GAGTTGAAGAGTCCAAGTCACTCATTAACTGACAGGAAATACCTGTAAACAGAG
ATTATATCAAGGCTTGTTAGCTAAGCAAATCTTTGATAAGTGTACCCTGTTAACA
TTTTAACCTTTCATTTTTTTTCCAGGAAATGAAAGCATCTTGCTGTTTATGGTTAT
AATTTCCAACAAACTAATCAGTACAGTAACCAATGGGAGAATAGAAATTTATTTC
TAGCATTCCACTGAATGGACAATGTCTGCTAAATATGAGCTTTTATTTCAGGATT
AATTTGTGATGGAAAAATAAATAGATTTTGCAAAGGTATATTGCTAAATAAATTT
TAGTACGAAAGTAACATAGTAGAATTGAACTGACTCAATTCAACATTAAAATATC
CTGTCATTTTTTTCACATATTAAACATCCTAGCCTTAATAAAATTGATATATTATT
AATAAATACTCTAACGATTTTAGTATATGACTTTGTTTTCCTATACTTTTCTAAAA
AATATTTGGGAATTTTACCTTAATTAGGAAATTTT
SEQ ID NO: 15: gRNA sequence to knock in SNP rs9403383
TCTAATATTTGCCTTTTTATGGG
SEQ ID NO: 16: HDRT sequence to knock in SNP rs9403383
CCATAGACTGGGACATTATTAAGAAGTCATAGTATAGACCATTCTAATATTTGCC
TTTTTATGGGACAATATTTTGAAGATGTCAGCAACCAAAACAAATATGGCTATGT
TAATAATTGCTAATACTTATATTTAAA

### REFERENCES

1. Lumish, H. S., M. O'Reilly, and M. P. Reilly, Sex Differences in Genomic Drivers of Adipose Distribution and Related Cardiometabolic Disorders: Opportunities for Precision Medicine. Arterioscler Thromb Vasc Biol, 2020. 40(1): p. 45-60
2 Wang, Y., et al., Will all Americans become overweight or obese? estimating the progression and cost of the US obesity epidemic. Obesity (Silver Spring), 2008. 16(10): p. 2323-30
3. Ashwell, M., T. J. Cole, and A. K. Dixon, Obesity: new insight into the anthropometric classification of fat distribution shown by computed tomography. Br Med J (Clin Res Ed), 1985. 290(6483): p. 1692-4 PMC1416121.
4 Neeland, I. J., et al., Dysfunctional adiposity and the risk of prediabetes and type 2 diabetes in obese adults. JAMA, 2012. 308(11): p. 1150-9 PMC3556508.
5. Mahabadi, A. A., et al., Association of pericardial fat, intrathoracic fat, and visceral abdominal fat with cardiovascular disease burden: the Framingham Heart Study. Eur Heart J, 2009. 30(7): p. 850-6 PMC3693564.
6 Britton, K. A., et al., Body fat distribution, incident cardiovascular disease, cancer, and all-cause mortality. J Am Coll Cardiol, 2013. 62(10): p. 921-5 PMC4142485.
7. Pi-Sunyer, F. X., The epidemiology of central fat distribution in relation to disease. Nutr Rev, 2004. 62(7 Pt 2): p. S120-6
8. Janssen, I., P. T. Katzmarzyk, and R. Ross, Waist circumference and not body mass index explains obesity-related health risk. Am J Clin Nutr, 2004. 79(3): p. 379-84
9. Neeland, I. J., et al., Associations of visceral and abdominal subcutaneous adipose tissue with markers of cardiac and metabolic risk in obese adult. Obesity (Silver Spring), 2013. 21(9): p. E439-47 PMC3751977.

10. Ramis, J. M., et al., Depot- and gender-related differences in the lipolytic pathway of adipose tissue from severely obese patients. Cell Physiol Biochem, 2006. 17(3-4): p. 173-80
11. Wajchenberg, B. L., Subcutaneous and visceral adipose tissue: their relation to the metabolic syndrome. Endocr Rev, 2000. 21(6): p. 697-738
12. Hajer, G. R., T. W. van Haeften, and F. L. Visseren, Adipose tissue dysfunction in obesity, diabetes, and vascular diseases. Eur Heart J, 2008. 29(24): p. 2959-71
13. Yang, Y. K., et al., Human mesenteric adipose tissue plays unique role versus subcutaneous and omental fat in obesity related diabetes. Cell Physiol Biochem, 2008. 22(5-6): p. 531-8
14. Chowdhury, B., et al., A multicompartment body composition technique based on computerized tomography. Int J Obes Relat Metab Disord, 1994. 18(4): p. 219-34
15. Hoffstedt, J., et al., Variation in adrener gic regulation of lipolysis between omental and subcutaneous adipocytes from obese and non-obese men. J Lipid Res, 1997. 38(4): p. 795-804
16. Frayn, K. N., Adipose tissue as a buffer for daily lipid flux. Diabetologia, 2002. 45(9): p. 1201-10
17. Snijder, M. B., et al., Low subcutaneous thigh fat is a risk factor for unfavourable glucose and lipid levels, independently of high abdominal fat. The Health ABC Study. Diabetologia, 2005. 48(2): p. 301-8
18. Seidell, J. C., et al., Waist and hip circumferences have independent and opposite effects on cardiovascular disease risk factors: the Quebec Family Study. Am J Clin Nutr, 2001. 74(3): p. 315-21
19. Buemann, B., et al., Possible role of adiponectin and insulin sensitivity in mediating the favorable effects of lower body fat mass on blood lipids. J Clin Endocrinol Metab, 2006. 91(5): p. 1698-704
20. Cereijo, R., M. Giralt, and F. Villarroya, Thermogenic brown and beige/brite adipogenesis in humans. Ann Med, 2015. 47(2): p. 169-77
21. Symonds, M. E., et al., Recent advances in our understanding of brown and beige adipose tissue: the good fat that keeps you healthy. F1000Res, 2018. 7 PMC6058473.
22. Finlin, B. S., et al., Human adipose beiging in response to cold and mirabegron. JCI Insight, 2018. 3(15) PMC6129119.
23. van Marken Lichtenbelt, W. D., et al., Cold-activated brown adipose tissue in healthy men. N Engl J Med, 2009. 360(15): p. 1500-8
24. Kriszt, R., et al., Optical visualisation of thermogenesis in stimulated single-cell brown adipocytes. Sci Rep, 2017. 7(1): p. 1383 PMC5431191.
25. Cypess, A. M., et al., Identification and importance of brown adipose tissue in adult humans. N Engl J Med, 2009. 360(15): p. 1509-17 PMC2859951.
26. Virtanen, K. A., et al., Functional brown adipose tissue in healthy adults. N Engl J Med, 2009. 360(15): p. 1518-25
27. He, Q., et al., Sex and race differences in fat distribution among Asian, African-American, and Caucasian prepubertal children. J Clin Endocrinol Metab, 2002. 87(5): p. 2164-70
28. Taylor, R. W., et al., Sex differences in regional body fat distribution from pre- to postpuberty. Obesity (Silver Spring), 2010. 18(7): p. 1410-6
29. Kotani, K., et al., Sexual dimorphism of age-related changes in whole-body fat distribution in the obese. Int J Obes Relat Metab Disord, 1994. 18(4): p. 207-2
30. Havel, P. J., et al., Gender differences in plasma leptin concentrations. Nat Med, 1996. 2(9): p. 949-50
31. Macotela, Y., et al., Sex and depot differences in adipocyte insulin sensitivity and glucose metabolism. Diabetes, 2009. 58(4): p. 803-12 PMC2661589.
32. Ding, J., et al., The association of pericardial fat with incident coronary heart disease: the Multi-Ethnic Study of Atherosclerosis(MESA). Am J Clin Nutr, 2009. 90(3): p. 499-504 PMC2728641.
33. Meisinger, C., et al., Body fat distribution and risk of type 2 diabetes in the general population: are there differences between men and women? The MONICA/KORA Augsburg cohort study. Am J Clin Nutr, 2006. 84(3): p. 483-9
34. de Koning, L., et al., Waist circumference and waist-to-hip ratio as predictors of cardiovascular events: meta-regression analysis of prospective studies. Eur Heart J, 2007. 28(7): p. 850-6
35. Viguerie, N., et al., Determinants of human adipose tissue gene expression: impact of diet, sex, metabolic status, and cis genetic regulation. PLoS Genet, 2012. 8(9): p. e1002959 PMC3459935.
36. Gomez-Abellan, P., et al., Sexual dimorphism in clock genes expression in human adipose tissue. Obes Surg, 2012. 22(1): p. 105-12 PMC4426975.
37. Shungin, D., et al., New genetic loci link adipose and insulin biology to body fat distribution. Nature, 2015. 518(7538): p. 187-196 PMC4338562.
38. Winkler, T. W., et al., The Influence of Age and Sex on Genetic Associations with Adult Body Size and Shape: A Large-Scale Genome-Wide Interaction Study. PLoS Genet, 2015. 11(10): p. e1005378 PMC4591371.
39. Rask-Andersen, M., et al., Genome-wide association study of body fat distribution identifies adiposity loci and sex-specific genetic effects. Nat Commun, 2019. 10(1): p. 339 PMC6341104.
40. Mogha, A., et al., Gpr 126/Adgrg6 Has Schwann Cell Autonomous and Nonautonomous Functions in Peripheral Nerve Injury and Repair. J Neurosci, 2016. 36(49): p. 12351-12367 PMC5148226.
41. Mogha, A., et al., Gpr126 functions in Schwann cells to control differentiation and myelination via G-protein activation. J Neurosci, 2013. 33(46): p. 17976-85 PMC3828454.
42. Petersen, S. C., et al., The adhesion GPCR GPR126 has distinct, domain-dependent functions in Schwann cell development mediated by interaction with laminin-211. Neuron, 2015. 85(4): p. 755-69 PMC4335265.
43. Liu, Z., et al., Dysregulation of STAT3 signaling is associated with endplate-oriented herniations of the intervertebral disc in Adgrg6 mutant mice. PLoS Genet, 2019. 15(10): p. e1008096 PMC6834287.
44. Liu, Z., et al., An adhesion G protein-coupled receptor is required in cartilaginous and dense connective tissues to maintain spine alignment. Elife, 2021. 10 PMC8328515.
45. Geng, F. S., et al., Semicircular canal morphogenesis in the zebrafish inner ear requires the function of gpr 126 (lauscher), an adhesion class G protein-coupled receptor gene. Development, 2013. 140(21): p. 4362-74 PMC4007713.
46. D′Amato, G., et al., Sequential Notch activation regulates ventricular chamber development. Nat Cell Biol, 2016. 18(1): p. 7-20 PMC4816493.
47. Torregrosa-Carrion, R., et al., Adhesion G protein-coupled receptor Gpr 126/Adgrg6 is essential for placental development. Sci Adv, 2021. 7(46): p. eabj5445 PMC8589310.
48. Suchy, T., et al., The repertoire of Adhesion G protein-coupled receptors in adipocytes and their functional relevance. Int J Obes (Lond), 2020. 44(10): p. 2124-2136 PMC7508673.
49. Makki, N., et al., Genomic characterization of the adolescent idiopathic scoliosis-associated transcriptome and regulome. Hum Mol Genet, 2021. 29(22): p. 3606-3615 PMC7823110.
50. Watts, G., UK Biobank opens it data vaults to researchers. BMJ, 2012. 344: p. e2459
51. Allen, N. E., et al., UK biobank data: come and get it. Sci Transl Med, 2014. 6(224): p. 224ed4
52. Justice, A. E., et al., Genome-wide meta-analysis of 241,258 adults accounting for smoking behaviour identifies novel loci for obesity traits. Nat Commun, 2017. 8: p. 14977 PMC5414044.
53. Consortium, G. T., The Genotype-Tissue Expression (GTEx) project. Nat Genet, 2013. 45(6): p. 580-5 PMC4010069.
54. Mikkelsen, T. S., et al., Comparative epigenomic analysis of murine and human adipogenesis. Cell, 2010. 143(1): p. 156-69 PMC2950833.
55. Messeguer, X., et al., PROMO: detection of known transcription regulatory elements using species-tailored searches. Bioinformatics, 2002. 18(2): p. 333-4
56. Wingender, E., The TRANSFAC project as an example of framework technology that supports the analysis of genomic regulation. Brief Bioinform, 2008. 9(4): p. 326-32
57. Matys, V., et al., TRANSFAC and its module TRANSCompel: transcriptional gene regulation in eukaryotes. Nucleic Acids Res, 2006. 34 (Database issue): p. D108-10 PMC1347505.
58. Gulyaeva, O., et al., Sox9-Meis1 Inactivation Is Required for Adipogenesis, Advancing Pref-1(+) to PDGFRalpha(+) Cells. Cell Rep, 2018. 25(4): p. 1002-1017 e4 PMC6903418.
59. Schindelin, J., et al., Fiji: an open-source platform for biological-image analysis. Nat Methods, 2012. 9(7): p. 676-82 PMC3855844.
60. Paavola, K. J., et al., Type IV collagen is an activating ligand for the adhesion G protein-coupled receptor GPR126. Sci Signal, 2014. 7(338): p. ra76 PMC4159047.
61. Petersen, R. K., et al., Cyclic AMP (CAMP)-mediated stimulation of adipocyte differentiation requires the synergistic action of Epac-and cAMP-dependent protein kinase-dependent processes. Mol Cell Biol, 2008. 28(11): p. 3804-16 PMC2423297.
62. Li, F., et al., Protein kinase A suppresses the differentiation of 3T3-L1 preadipocytes. Cell Res, 2008. 18(2): p. 311-23
63. Steger, D. J., et al., Propagation of adipogenic signals through an epigenomic transition state. Genes Dev, 2010. 24(10): p. 1035-44 PMC2867208.
64. Karner, C. M., et al., Gpr 126/Adgrg6 deletion in cartilage models idiopathic scoliosis and pectus excavatum in mice. Hum Mol Genet, 2015. 24(15): p. 4365-73 PMC4492399.
65. Shin, S., et al., Dynamic control of adipose tissue development and adult tissue homeostasis by platelet-derived growth factor receptor alpha. Elife, 2020. 9 PMC7338051.
66. Sun, C., et al., Mosaic Mutant Analysis Identifies PDGFRalpha/PDGFRbeta as Negative Regulators of Adipogenesis. Cell Stem Cell, 2020. 26(5): p. 707-721 e5 PMC7214206.
67. Joshi, P. A., et al., PDGFRalpha(+) stromal adipocyte progenitors transition into epithelial cells during lobulo-alveologenesis in the murine mammary gland. Nat Commun, 2019. 10(1): p. 1760 PMC6465250.
68. Berry, R. and M. S. Rodeheffer, Characterization of the adipocyte cellular lineage in vivo. Nat Cell Biol, 2013. 15(3): p. 302-8 PMC3721064.
69. Hu, E., P. Liang, and B. M. Spiegelman, AdipoQ is a novel adipose-specific gene dysregulated in obesity. J Biol Chem, 1996. 271(18): p. 10697-703
70. Shan, T., et al., Lkb1 controls brown adipose tissue growth and thermogenesis by regulating the intracellular localization of CRTC3. Nat Commun, 2016. 7: p. 12205 PMC4974456.
71. Zhang, X., et al., A bone-specific adipogenesis pathway in fat-free mice defines key origins and adaptations of bone marrow adipocytes with age and disease. Elife, 2021. 10 PMC8412938.
72. Bartoletti, G., et al., Pdgfra regulates multipotent cell differentiation towards chondrocytes via inhibiting Wnt9a/beta-catenin pathway during chondrocranial cartilage development. Dev Biol, 2020. 466(1-2): p. 36-46 PMC7494641.
73. Navarro Gonzalez, J., et al., The UCSC Genome Browser database: 2021 update. Nucleic Acids Res, 2021. 49(D1): p. D1046-D1057 PMC7779060.
74. Elsukova, E. I., L. N. Medvedev, and O. V. Mizonova, Physiological Features of Perigonadal Adipose Tissue Containing Uncoupling Protein UCPI in ICR Mice. Bull Exp Biol Med, 2016. 161(3): p. 347-50
75. Takahashi, G., et al., GONAD: Genome-editing via Oviductal Nucleic Acids Delivery system: a novel microinjection independent genome engineering method in mice. Sci Rep, 2015. 5: p. 11406 PMC4476150.
76. Bates, R., W. Huang, and L. Cao, Adipose Tissue: An Emerging Target for Adeno-associated Viral Vectors. Mol Ther Methods Clin Dev, 2020. 19: p. 236-249 PMC7566077.
77. Horlbeck, M. A., et al., Nucleosomes impede Cas9 access to DNA in vivo and in vitro. Elife, 2016. 5 PMC4861601.
78. Simeonov, D. R., et al., Discovery of stimulation-responsive immune enhancers with CRISPR activation. Nature, 2017. 549(7670): p. 111-115 PMC5675716.
79. Matharu, N., et al., CRISPR-mediated activation of a promoter or enhancer rescues obesity caused by haploinsufficiency. Science, 2019. 363(6424) PMC6570489.
80. Randall, J. C., et al., Sex-stratified genome-wide association studies including 270,000 individuals show sexual dimorphism in genetic loci for anthropometric traits. PLoS Genet, 2013. 9(6): p. e1003500 PMC3674993 Stefansson, Valgerdur Steinthorsdottir, and Gudmar Thorleifsson are employed by deCODE Genetics with stock/stock options in the company. Ines Barroso and spouse own stock in Incyte Ltd and GlaxoSmithKline. Nilesh J Samani holds a Chair supported by the British Heart Foundation and is an NIHR Senior Investigator.
81. Ng, M. C. Y., et al., Discovery and fine-mapping of adiposity loci using high density imputation of genome-wide association studies in individuals of African ancestry: African Ancestry Anthropometry Genetics Consortium. PLoS Genet, 2017. 13(4): p. e1006719 PMC5419579.
82. Thomas, J. J., et al., Trends in Incidence of Adolescent Idiopathic Scoliosis: A Modern US Population-based Study. J Pediatr Orthop, 2021. 41(6): p. 327-332 PMC8542350.
83. Xu, J. F., et al., Association of GPR126 gene polymorphism with adolescent idiopathic scoliosis in Chinese populations. Genomics, 2015. 105(2): p. 101-7
84. Kou, I., et al., Genetic variants in GPR126 are associated with adolescent idiopathic scoliosis. Nat Genet, 2013. 45(6): p. 676-9
85. Virtanen, K. A., et al., Glucose uptake and perfusion in subcutaneous and visceral adipose tissue during insulin stimulation in nonobese and obese humans. J Clin Endocrinol Metab, 2002. 87(8): p. 3902-10
86. Duan, Y., et al., Sexual dimorphism in vertebral fragility is more the result of gender differences in age-related bone gain than bone loss. J Bone Miner Res, 2001. 16(12): p. 2267-75
87. Pantoja, C., J. T. Huff, and K. R. Yamamoto, Glucocorticoid signaling defines a novel commitment state during adipogenesis in vitro. Mol Biol Cell, 2008. 19(10): p. 4032-41 PMC2555927.
88. Ou, C. Y., et al., Coregulator cell cycle and apoptosis regulator 1 (CCARI) positively regulates adipocyte differentiation through the glucocorticoid signaling pathway. J Biol Chem, 2014. 289(24): p. 17078-86 PMC4059149.
89. Furuhata, R., et al., Progesterone receptor membrane associated component 1 enhances obesity progression in mice by facilitating lipid accumulation in adipocytes. Commun Biol, 2020. 3(1): p. 479 PMC7473863.
90. O'Brien, S. N., et al., Identification of progesterone receptor in human subcutaneous adipose tissue. J Clin Endocrinol Metab, 1998. 83(2): p. 509-13
91. Lacasa, D., et al., Progesterone stimulates adipocyte determination and differentiation 1/sterol regulatory element-binding protein 1c gene expression. potential mechanism for the lipogenic effect of progesterone in adipose tissue. J Biol Chem, 2001. 276(15): p. 11512-6
92. Chau, Y. Y., et al., Visceral and subcutaneous fat have different origins and evidence supports a mesothelial source. Nat Cell Biol, 2014. 16(4): p. 367-75 PMC4060514.
93. Zhou, B., et al., Epicardial progenitors contribute to the cardiomyocyte lineage in the developing heart. Nature, 2008. 454(7200): p. 109-13 PMC2574791.
94. Matharu, N. and N. Ahituv, Modulating gene regulation to treat genetic disorders. Nat Rev Drug Discov, 2020. 19(11): p. 757-775
95. Shukla, L., et al., Fat Therapeutics: The Clinical Capacity of Adipose-Derived Stem Cells and Exosomes for Human Disease and Tissue Regeneration. Front Pharmacol, 2020. 11: p. 158 PMC7062679.
96. Shoji, T., et al., Local transplantation of human multipotent adipose-derived stem cells accelerates fracture healing via enhanced osteogenesis and angiogenesis. Lab Invest, 2010. 90(4): p. 637-49
97. Liu, X., et al., Brown Adipose Tissue Transplantation Reverses Obesity in Ob/Ob Mice. Endocrinology, 2015. 156(7): p. 2461-9
98. White, J. D., R. S. Dewal, and K. I. Stanford, The beneficial effects of brown adipose tissue transplantation. Mol Aspects Med, 2019. 68: p. 74-81 PMC6708446.
99. Sudlow, C., et al., UK biobank: an open access resource for identifying the causes of a wide range of complex diseases of middle and old age. PLoS Med, 2015. 12(3): p. e1001779 PMC4380465.
100. Loh, P. R., et al., Reference-based phasing using the Haplotype Reference Consortium panel. Nat Genet, 2016. 48(11): p. 1443-1448 PMC5096458.
101. Das, S., et al., Next-generation genotype imputation service and methods. Nat Genet, 2016. 48(10): p. 1284-1287 PMC5157836.
102. Chang, C. C., et al., Second-generation PLINK: rising to the challenge of larger and richer datasets. GigaScience, 2015. 4: p. 7 4342193.
103. Nguyen, H. P., et al., Aifm2, a NADH Oxidase, Supports Robust Glycolysis and Is Required for Cold-and Diet-Induced Thermogenesis. Mol Cell, 2020. 77(3): p. 600-617 e4 PMC7031813.
104. Ushiki, A., et al., Deletion of CTCF sites in the SHH locus alters enhancer-promoter interactions and leads to acheiropodia. Nat Commun, 2021. 12(1): p. 2282 PMC8052326.
105. Doench, J. G., et al., Optimized sgRNA design to maximize activity and minimize off-target effects of CRISPR-Cas9. Nat Biotechnol, 2016. 34(2): p. 184-191 PMC4744125.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

What is claimed is:

1. A method of reducing body fat in a human male subject, the method comprising mutating or reducing the expression of an Adgrg6 gene in one or more cells in the human male subject.

2. The method of claim 1, wherein the mutating the Adgrg6 gene comprises knocking out the gene or introducing a nucleotide deletion or insertion into the gene.

3. The method of claim 2, wherein knocking out the Adgrg6 gene comprises introducing into the one or more cells of the human male subject a nuclease targeted to the Adgrg6 gene.

4. The method of claim 3, wherein the nuclease is a RNA-guided nuclease, a zinc finger nuclease (ZFN), or a transcription activator-like effector nuclease (TALEN).

5. The method of claim 4, wherein the RNA-guided nuclease is a clustered regularly interspaced short palindromic repeats (CRISPR) nuclease and the method further comprises introducing into the one or more cells of the human male subject a guide RNA (gRNA) that targets a portion of the Adgrg6 gene, wherein the gRNA comprises a sequence having at least 90%, 95%, 98%, 99% or 100% identity to a sequence of
(SEQ ID NO: 1)
ATCGAATGTGGAGCTGCCAT
or
(SEQ ID NO: 2)
GGACAAAACCACTCGGCAGT.

6. The method of claim 1, wherein the reducing the expression of the Adgrg6 gene comprises CRISPR interference (CRISPRi), RNA interference (RNAi), or antisense therapy.

7. The method of claim 6, wherein the CRISPRi comprises introducing into the one or more cells of the human male subject a catalytically-inactive nuclease and a gRNA that targets a portion of a promoter or enhancer sequence operably linked to a coding sequence of the Adgrg6 gene.

8. The method of claim 7, wherein the catalytically-inactive nuclease is linked to a transcriptional repressor domain.

9. The method of claim 7 or 8, wherein the catalytically-inactive nuclease is dCas9.

10. The method of any one of claims 7 to 9, wherein the gRNA targets the promoter sequence comprising the sequence of SEQ ID NO:8.

11. The method of claim 10, wherein the gRNA comprises a sequence having at least 90%, 95%, 98%, 99% or 100% identity to a sequence of
(SEQ ID NO: 3)
AGCTGAGGAAGTAGGGTGTGCGTGGG,
(SEQ ID NO: 4)
GGCGGCAGGTCCCTCCTCGCAGGGAA,
(SEQ ID NO: 5)
CCCTCCTCGCAGGGAAGTTGGCAGGG,
(SEQ ID NO: 6)
CCTCGCAGGGAAGTTGGCAGGGTGAG,
or
(SEQ ID NO: 7)
CCCTCCTCGCAGGGAAGTTGGCAGGG.

12. The method of any one of claims 7 to 9, wherein the gRNA targets the enhancer sequence comprising the sequence of SEQ ID NO:14.

13. The method of claim 12, wherein gRNA comprises a sequence having at least 90%, 95%, 98%, 99% or 100% identity to a sequence of
(SEQ ID NO: 9)
CCCGTAACATGGGATCTATGGTGTAG,
(SEQ ID NO: 10)
CCCAGAATGCAGGAAGTGCACCATTC,
(SEQ ID NO: 11)
CCCCTGAATTAAAGACAGTCACCCAG,
(SEQ ID NO: 12)
GTCTATGGTGAAAAGAGACCCCTGAA,
or
(SEQ ID NO: 13)
CTCTAATACCAAACTTTCCAAGCTCC.

14. The method of claim 1, wherein reducing the expression of the Adgrg6 gene comprises knocking in a single nucleotide polymorphism (SNP) proximal to the Adgrg6 gene in one or more cells of the human male subject, wherein the SNP is rs9403383.

15. The method of claim 14, wherein the knocking in comprises introducing into one or more cells of the human male subject a gRNA, an RNA-guided nuclease, and a homology-directed-repair template (HDRT) comprising the SNP rs9403383.

16. The method of claim 15, wherein the gRNA comprises a sequence having at least 90%, 95%, 98%, 99% or 100% identity to the sequence of
(SEQ ID NO: 15)
TCTAATATTTGCCTTTTTATGGG.

17. The method of 15 or 16, wherein the HDRT comprises a sequence having at least 90%, 95%, 98%, 99% or 100% identity to the sequence of
(SEQ ID NO: 16)
CCATAGACTGGGACATTATTAAGAAGTCATAGTATAGACCATTCTAATA
TTTGCCTTTTTATGGGACAATATTTTGAAGATGTCAGCAACCAAAACAA
ATATGGCTATGTTAATAATTGCTAATACTTATATTTAAA.

18. A method of reducing body fat in a human male subject, the method comprising reducing or blocking the activity of the Adhesion G-protein coupled receptor G6 (ADGRG6) protein in one or more cells in the human male subject.

19. The method of claim 18, wherein the method comprises administering to the human male subject a small molecule that binds to the ADGRG6 protein.

20. The method of claim 18 or 19, wherein the small molecule is selected from the group consisting of valproic acid, 4-(5-benzo(1,3)dioxol-5-yl-4-pyridin-2-yl-1H-imidazol-2-yl)benzamide, dorsomorphin, tetrachlorodibenzodioxin, acetaminophen, benzo(a)pyrene, bisphenol A, estradiol, tretinoin, and trichostatin A.

21. The method of claim 18 or 19, wherein the small molecule is selected from the group consisting of alfuzosin, terazosin, clonidine, bisoprolol, betaxolol, metoprolol, atenolol, albuterol, nadolol, penbutolol, tolterodine, atropine, scopolamine, calcimar, metoclopramide, haloperidol, olanzapine, ropinirole, pramipexole, loratadine, cetirizine, demenhydrinate, cimetidine, ranitidine, trazodone, sumatriptan, exenatide, fentanyl, codein, meperidine, oxycodone, montelukast, misoprostol, clopidogrel, aripiprazole, quetiapine, montelukast, olanzapine, and valsartan.

22. The method of any one of claims 1 to 21, wherein the human male subject has or is at risk of developing a metabolic disease.

23. The method of claim 22, wherein the metabolic disease is obesity, Type-1 diabetes, Type-2 diabetes, or a cardiovascular disease.

24. The method of claim 23, wherein the obesity is diet-induced obesity.

25. The method of any one of claims 1 to 24, wherein the human male subject is overweight.

26. The method of any one of claims 1 to 25, wherein the human male subject is undergoing a sex reassignment therapy to change into a female subject.

27. The method of any one of claims 1 to 26, wherein the method reduces VAT in the human male subject.

28. The method of any one of claims 1 to 27, wherein the method reduces body weight.

29. The method of any one of claims 1 to 28, wherein the method does not reduce lean mass.

30. The method of any one of claims 1 to 29, wherein the method reduces blood glucose.

31. The method of any one of claims 1 to 30, wherein the method increases insulin sensitivity.

32. The method of any one of claims 1-31, wherein the method comprises mutating or reducing the expression of an Adgrg6 gene in one or more cells ex vivo and then introducing the cells into the human male subject.

33. The method of claim 32, further comprising before the mutating or reducing, isolating the cells from the human male subject.

34. The method of claim 32 or 33, wherein the cells are adipose stem cells or adipose progenitor cells.

35. An isolated adipose cell having a mutated Adgrg6 gene or an Adgrg6 gene with reduced expression compared to a wild-type adipose cell.

36. The cell of claim 35, wherein the isolated adipose cell comprises a catalytically-inactive nuclease and a gRNA that targets a portion of a promoter or enhancer sequence operably linked to a coding sequence of the Adgrg6 gene.

37. The cell of claim 36, wherein the catalytically-inactive nuclease is linked to a transcriptional repressor domain.

38. The cell of claim 36 or 37, wherein the catalytically-inactive nuclease is dCas9.

39. The cell of any one of claims 36 to 38, wherein the gRNA targets the promoter sequence comprising the sequence of SEQ ID NO:8.

40. The cell of claim 39, wherein the gRNA comprises a sequence having at least 90%, 95%, 98%, 99% or 100% identity to a sequence of any one of SEQ ID NOS: 3-7.

41. The cell of any one of claims 36 to 38, wherein the gRNA targets the enhancer sequence comprising the sequence of SEQ ID NO: 14.

42. The cell of claim 41, wherein the gRNA comprises a sequence having at least 90%, 95%, 98%, 99% or 100% identity to a sequence of any one of SEQ ID NOS: 9-13.

43. The cell of any one of claims 35 to 42, wherein the isolated adipose cell is an adipose stem cell or progenitor cell.

44. A composition comprising a guide RNA (gRNA), wherein the gRNA comprises a sequence having at least 90%, 95%, 98%, 99% or 100% identity to a sequence of any one of SEQ ID NOS: 3-7 and 9-13.

45. The composition of claim 44, wherein the composition further comprises a catalytically-inactive nuclease.

46. The composition of claim 45, wherein the catalytically-inactive nuclease is linked to a transcriptional repressor domain.

47. The composition of claim 45 or 46, wherein the catalytically-inactive nuclease is dCas9.
